(19) United States
(12) Patent Application Publication
(10) Pub. No.: US 2021/0238644 A1
(43) Pub. Date: Aug. 5, 2021

(54) CELL CULTURE STRATEGIES FOR MODULATING PROTEIN GLYCOSYLATION

(71) Applicant: GENENTECH, INC., South San Francisco, CA (US)

(72) Inventors: Inn Huam YUK, South San Francisco, CA (US); Patrick Daniel AHYOW, South San Francisco, CA (US); Anh Thuy NGUYEN DANG, South San Francisco, CA (US); Melissa Siu-Lan MUN, South San Francisco, CA (US); Kristen Leigh DOUGLAS, South San Francisco, CA (US); Robert David KISS, South San Francisco, CA (US)

(73) Assignee: GENENTECH, INC., South San Francisco, CA (US)

(21) Appl. No.: 17/172,528

(22) Filed: Feb. 10, 2021

Related U.S. Application Data
(63) Continuation of application No. PCT/US2019/045900, filed on Aug. 9, 2019.
(60) Provisional application No. 62/717,751, filed on Aug. 10, 2018.

(51) Int. Cl. C12P 21/00; C07K 16/28; C12N 5/00

(52) U.S. Cl. CPC C12P 21/005; C07K 16/2887; C07K 2317/14; C07K 2317/41; C12N 5/0018

(57) ABSTRACT

The presently disclosed subject matter relates to cell culture media and cell culture strategies for modulating the glycosylation pattern, e.g., fucosylation and/or galactosylation pattern, of a glycoprotein of interest, e.g., an antibody, as well as cell culture and glycoprotein compositions prepared using such media and/or strategies.

### CROSS-REFERENCE TO RELATED APPLICATIONS

This application is a continuation of International Patent Application No. PCT/US2019/045900, filed Aug. 9, 2019, which claims priority to U.S. Provisional Patent Application Ser. No. 62/717,751, filed Aug. 10, 2018, the contents of each of which are hereby incorporated by reference in their entireties.

### TECHNICAL FIELD

The presently disclosed subject matter relates to cell culture media and cell culture strategies for modulating the glycosylation pattern, e.g., fucosylation and/or galactosylation pattern, of a glycoprotein of interest, e.g., an antibody, as well as cell culture and glycoprotein compositions prepared using such media and/or strategies.

### BACKGROUND

N-linked glycosylation can impact the physiochemical properties of recombinant glycoproteins, including monoclonal antibodies (mAbs). These properties include protein folding, solubility, binding, stability, immunogenicity, and pharmacokinetics (Varki A. (1993), Glycobiology, 3 (2), 97-130). Depending on the mechanism of action for a therapeutic mAb, the potency of the mAb can depend on complement-dependent cytotoxicity (CDC) activity and/or antibody-dependent cell-mediated cytotoxicity (ADCC) activity. In some studies, mAbs with higher terminal galactosylation, which refers to the addition of a terminal galactose residue to N-acetyl-glucosamine (GlcNAc), have higher CDC activity (Boyd et al., (1995) Mol. Immunology 32, 1311-1318; Hodoniczky et al., (2005), Biotechnol. Prog. 21, 1644-1652; Tsuchiya et al., (1989) J. Rheumatol., 16, 285-290). Therefore, an optimal and consistent level of galactosylation can be highly desirable for a mAb product with CDC as a mechanism of action. In other studies, mAbs with lower core fucosylation, which refers to the addition of a fucose residue to oligosaccharide core, have higher ADCC activity (Ferrara et al., (2011), Proc. Natl. Acad. Sci., 108, 12669-12674; Shields et al., (2002), J. Biol. Chem., 277, 26733-26740; Shinkawa et al., (2003) J. Biol. Chem., 278, 3466-3473; Thomann et al., (2016) Molecular Immunology, 73, 60-75). Therefore, an optimal and consistent level of afucosylation (i.e., the lack of core fucose on the N-linked glycan) can be highly desirable for a mAb product with ADCC as a mechanism of action. Strategies to modulate mAb glycosylation (e.g., galactosylation and/or fucosylation) in cell culture processes generally belong to one of four categories: (1) genetic engineering of recombinant cell lines (Louie et al., (2016), Biotechnol Bioeng, 114 (3), 632-644; Yamane-Ohnuki et al., (2004), Biotechnol Bioeng, 87(5), 614-622); (2) addition of enzyme inhibitors (Allen et al., (2016), ACS Chem Biol, 11(10), 2734-2743; Okeley et al., (2013), PNAS, 110 (14), 5404-5409); (3) modifying the levels of co-factors and substrates for glycosylation, including supplementation with alternative sugars (Hossler et al., (2014), Biotechnol Prog, 30 (6), 1419-1431; Hossler et al., (2017), mAbs, 9 (4), 715-734); and (4) fine-tuning of culture process parameters (Konno et al., (2012), Cytotechnology, 64, 249-265). Although many cell culture process parameters are known to impact galactosylation (Hossler et al., (2009), Glycobiology, 19 (9), 936-949), fewer have been identified to control fucosylation.

### SUMMARY OF THE INVENTION

The subject matter disclosed herein relates to modulating the glycosylation pattern (e.g., galactosylation and/or fucosylation pattern(s)) of a recombinant glycoprotein of interest. For example, but not by way of limitation, the embodiments described herein relate to modulating glycosylation to achieve or preserve a desired glycoprotein glycosylation pattern (e.g., galactosylation and/or fucosylation pattern(s)). Methods by which glycosylation can be modulated in accordance with the instant disclosure include, but are not limited to: (1) control of cell culture media and/or cell culture manganese (Mn) concentration, e.g., with respect to Mn concentration analysis of raw materials, Mn supplementation to cell culture media and/or during cell culture, and/or minimizing Mn loss from cell culture by establishing a reduced pH target or set point for media pH adjustment prior to High Temperature Short Time (HTST) heat treatment of the media; (2) controlling process parameters during cell culture, e.g., pC0₂, media hold duration, culture duration, cultivation temperature and osmolality/Na⁺; and (3) control of cell culture media and/or cell culture galactose and/or fucose concentration. The subject matter of the present disclosure is also directed to cell culture and glycoprotein compositions prepared when such process parameters are controlled as described herein.

In certain embodiments, the present disclosure is directed to a method for modulating the glycosylation pattern of a glycoprotein of interest in a cell culture, comprising: modulating the following parameters, either alone or in any combination, in a cell culture medium, and/or, in a cell culture environment: a Mn concentration from about 1 nM to about 20000 nM in a high partial pressure CO₂ (pCO₂) condition; a Mn concentration from about 1 nM to about 30000 nM in a low pCO₂ condition; a pCO₂ from about 10 mmHg to about 250 mmHg; a pre-inoculation cell culture media hold duration from about 0 hrs to about 120 hrs at a temperature of about 25° C. to 39° C.; a cell culture duration from about 0 days to about 150 days; a Na+ concentration from about 0 mM to about 300 mM; an osmolality from about 250 mOsm/kg to about 550 mOsm/kg; a galactose concentration from about 0 mM to about 60 mM; a fucose concentration from about 0 mM to about 60 mM; and a cultivation temperature from about 29° C. to about 39° C.

In certain embodiments, the cell culture environment is in a bioreactor with or without cells. In certain embodiments, the low pCO₂ condition is from about 10 to about 100 mmHg, and the high pCO₂ condition is from about 20 to about 250 mmHg. In certain embodiments, the duration of pCO₂ modulation covers at least the first half of the cell culture duration.

In certain embodiments, the glycoprotein of interest is a recombinant protein. In certain embodiments, the recombinant protein is an antibody or antibody fragment, a scFv (single-chain variable fragment), BsDb (bispecific diabody), scBsDb (single-chain bispecific diabody), scBsTaFv (single-chain bispecific tandem variable domain), DNL-(Fab)3 (dock-and-lock trivalent Fab), sdAb (single-domain antibody) and BssdAb (bispecific single-domain antibody). In certain embodiments, the antibody is a chimeric, a humanized or a human antibody. In certain embodiments, the antibody is an anti-CD20 antibody. In certain embodiments, the anti-CD20 antibody is ocrelizumab. In certain embodiments, the antibody or antibody fragment exhibits: a % G0-F (percent afucosylated glycoprotein) between about 0% to about 20%; about 1% to about 15%; about 1% to about 10%; or about 1% to about 8%; or, a normalized % G0-F between about 0% to about 20%; about 1% to about 15%; about 1% to about 10%; or about 1% to about 8%; and/or, a % G0 (percent agalactosylated glycoprotein) between about 40% to about 90%; about 50% to about 90%; about 55% to about 85%; or about 60% to about 80%.

In certain embodiments, the glycosylation is modulated to achieve: an increased afucosylation (e.g., G0-F (afucosylated G0)), while decreasing agalactosylation (e.g., G0 (fucosylated, agalactosylated G0)); or, a decreased afucosylation (e.g., G0-F), while increasing agalactosylation (e.g., G0); or, an increased or decreased afucosylation (e.g., G0-F) without impacting agalactosylation (e.g., G0); or, an increased or decreased agalactosylation (e.g., G0) without impacting afucosylation (e.g., G0-F).

In certain embodiments, the step of modulating the glycosylation pattern of the glycoprotein of interest comprises: modulating the Mn concentration from about 1 nM to about 30000 nM under a low pCO₂ condition, or, modulating the Mn concentration from about 1 nM to about 20000 nM under a high pCO₂ condition, and the following parameters, either alone or in any combination, in the cell culture medium, and/or in the cell culture environment: the pre-inoculation cell culture media hold duration from about 0 hrs to about 120 hrs at a temperature of about 25° C. to 39° C.; the cell culture duration from about 0 days to about 150 days; the Na+ concentration from about 0 mM to about 300 mM; the osmolality from about 250 mOsm/kg to about 550 mOsm/kg; the galactose concentration from about 0 mM to about 60 mM; the fucose concentration from about 0 mM to about 60 mM; and the cultivation temperature from about 29° C. to about 39° C.

In certain embodiments, the step of modulating the glycosylation pattern of the glycoprotein of interest comprises: modulating the Mn concentration from about 1 nM to about 30000 nM under a low pCO₂ condition, or, modulating the Mn concentration from about 1 nM to about 20000 nM under a high pCO₂ condition, and the following parameters in the cell culture medium, and/or in the cell culture environment: the pre-inoculation cell culture media hold duration from about 0 hrs to about 120 hrs at a temperature of about 25° C. to 39° C.; and the cell culture duration from about 0 days to about 150 days.

In certain embodiments, the step of modulating the glycosylation pattern of the glycoprotein of interest comprises: modulating the Mn concentration from about 1 nM to about 30000 nM under a low pCO₂ condition, or, modulating the Mn concentration from about 1 nM to about 20000 nM under a high pCO₂ condition, and the following parameters in the cell culture medium, and/or in the cell culture environment: the galactose concentration from about 0 mM to about 60 mM; and/or, the fucose concentration from about 0 mM to about 60 mM.

In certain embodiments, the step of modulating the glycosylation pattern of the glycoprotein of interest comprises: modulating the pre-inoculation cell culture media hold duration and any of the following parameters, either alone or in any combination, in the cell culture medium, and/or in the cell culture environment: a Mn concentration from about 1 nM to about 20000 nM in a high partial pressure CO₂ (pCO₂) condition; a Mn concentration from about 1 nM to about 30000 nM in a low pCO₂ condition; a pCO₂ from about 10 mmHg to about 250 mmHg; a cell culture duration from about 0 days to about 150 days; a Na+ concentration from about 0 mM to about 300 mM; an osmolality from about 250 mOsm/kg to about 550 mOsm/kg; a galactose concentration from about 0 mM to about 60 mM; a fucose concentration from about 0 mM to about 60 mM; and a cultivation temperature from about 29° C. to about 39° C.; wherein the cell culture media hold duration is from about 0 hrs to about 120 hrs at a temperature of about 25° C. to 39° C.

In certain embodiments, the step of modulating the glycosylation pattern of the glycoprotein of interest comprises: modulating the pre-inoculation cell culture media hold duration and the following parameters in the cell culture medium, and/or in the cell culture environment: the Mn concentration from about 1 nM to about 20000 nM in a high partial pressure CO₂ (pCO₂) condition, or, a Mn concentration from about 1 nM to about 30000 nM in a low pCO₂ condition; the pCO₂ from about 10 mmHg to about 250 mmHg; and, the cell culture duration from about 0 days to about 150 days; wherein the cell culture media hold duration is from about 0 hrs to about 120 hrs at a temperature of about 25° C. to 39° C.

In certain embodiments, the step of modulating the glycosylation pattern of the glycoprotein of interest comprises: modulating the cell culture duration and any of the following parameters, either alone or in any combination, in the cell culture medium, and/or in the cell culture environment: a Na+ concentration from about 0 mM to about 300 mM; an osmolality from about 250 mOsm/kg to about 550 mOsm/kg; a galactose concentration from about 0 mM to about 60 mM; a fucose concentration from about 0 mM to about 60 mM; and a cultivation temperature from about 29° C. to about 39° C., wherein the cell culture duration is from about 0 days to about 150 days.

In certain embodiments, the step of modulating the glycosylation pattern of the glycoprotein of interest comprises: modulating the Na+ concentration of about 0 nM to about 300 nM and any of the following parameters, either alone or in any combination, in the cell culture medium, and/or in the cell culture environment: an osmolality from about 250 mOsm/kg to about 550 mOsm/kg; a galactose concentration from about 0 mM to about 60 mM; a fucose concentration from about 0 mM to about 60 mM; and a cultivation temperature from about 29° C. to about 39° C., wherein the Na+ concentration from about 0 mM to about 300 mM.

In certain embodiments, the step of modulating the glycosylation pattern of the glycoprotein of interest comprises: modulating the Na+ concentration and the pCO₂ from about 10 mmHg to about 250 mmHg.

In certain embodiments, the step of modulating the glycosylation pattern of the glycoprotein of interest comprises: modulating the osmolality and any of the following parameters, either alone or in any combination, in the cell culture medium, and/or in the cell culture environment: a galactose concentration from about 0 mM to about 60 mM; a fucose concentration from about 0 mM to about 60 mM; and a cultivation temperature from about 29° C. to about 39° C., wherein the osmolality is from about 250 mOsm/kg to about 550 mOsm/kg.

In certain embodiments, the step of modulating the glycosylation pattern of the glycoprotein of interest comprises: modulating the Mn concentration from about 1 nM to about 30000 nM under a low pCO₂ condition, or modulating the Mn concentration from about 1 nM to about 20000 nM under a high pCO₂ condition, modulating the Na+ concentration from about 0 mM to about 300 mM, and modulating the duration of the pre-inoculation cell culture media hold from about 0 hrs to about 120 hrs. In certain embodiments, modulation of the glycosylation pattern of the glycoprotein of interest comprises modulating the osmolality from about 250 mOsm/kg to about 550 mOsm/kg and the pCO₂ from about 10 mmHg to about 250 mmHg.

In certain embodiments, the modulation of the glycosylation pattern of the glycoprotein of interest comprises modulating: the cultivation temperature from about 29° C. to about 39° C., and, the galactose concentration from about 0 mM to about 60 mM; and/or the fucose concentration from about 0 mM to about 60 mM.

In certain embodiments, modulation of the glycosylation pattern of the glycoprotein of interest comprises modulating the pCO₂ from about 10 mmHg to about 250 mmHg and the fucose concentration from about 0 mM to about 60 mM.

In certain embodiments, modulation of the glycosylation pattern of the glycoprotein of interest comprises modulating the fucose concentration from about 0 mM to about 60 mM and the cultivation temperature from about 29° C. to about 39° C.

In certain embodiments, the modulation of the glycosylation pattern of the glycoprotein of interest comprises: modulating a pCO₂ concentration and any of the following parameters, either alone or in any combination, in the cell culture medium, and/or in the cell culture environment: a Mn concentration from about 1 nM to about 20000 nM in a high partial pressure CO₂ (pCO₂) condition; a Mn concentration from about 1 nM to about 30000 nM in a low pCO₂ condition; a pre-inoculation cell culture media hold duration from about 0 hrs to about 120 hrs at a temperature of about 25° C. to 39° C.; a cell culture duration from about 0 days to about 150 days; a Na+ concentration from about 0 mM to about 300 mM; an osmolality from about 250 mOsm/kg to about 550 mOsm/kg; a galactose concentration from about 0 mM to about 60 mM; a fucose concentration from about 0 mM to about 60 mM; and a cultivation temperature from about 29° C. to about 39° C., wherein the pCO₂ concentration is from about 10 mmHg to about 250 mmHg.

In certain embodiments, the Mn concentration is from about 1 nM to about 20000 nM in a high pCO₂ culture; from about 1 nM to about 10000 nM, from about 1 nM to about 5000 nM, from about 1 nM to about 4000 nM, from about 1 nM to about 3000 nM, from about 1 nM to about 2000 nM, from about 1 nM to about 1000 nM in a high pCO₂ culture; from about 1 nM to about 500 nM, from about 1 nM to about 100 nM, from about 1 nM to about 50 nM, from about 1 nM to about 20 nM, from about 20 nM to about 2000 nM, from about 20 nM to about 3000 nM, from about 20 nM to about 10000 nM, from about 20 nM to about 20,000 nM, from about 20 nM to about 300 nM, about 30 nM to about 110 nM in a high pCO₂ culture.

In certain embodiments, the Mn concentration is about 1 nM to about 30000 nM in a low pCO₂ culture; from about 1 nM to about 20000 nM; from about 1 nM to about 10000 nM, from about 1 nM to about 5000 nM, from about 1 nM to about 4000 nM, from about 1 nM to about 3000 nM, from about 1 nM to about 2000 nM, from about 1 nM to about 1000 nM; from about 1 nM to about 500 nM, from about 1 nM to about 100 nM, from about 1 nM to about 50 nM, from about 1 nM to about 20 nM, from about 20 nM to about 100 nM, about 20 nM to about 300 nM, from about 20 nM to about 500 nM, from about 20 nM to about 1000 nM, from about 20 nM to about 2000 nM, from about 20 nM to about 3000 nM, from about 20 nM to about 5000 nM, from about 20 nM to about 10000 nM, from about 20 nM to about 20000 nM, or about 30 nM to about 110 nM in a low pCO₂ culture.

In certain embodiments, modulation of the Mn concentration comprises determining the Mn content in cell culture raw materials and selecting raw material lots to modulate the Mn concentration.

In certain embodiments, modulation of the Mn concentration comprises (i) controlling materials that come into contract with cell culture media or cell culture; or (ii) accounting for the concentration of leached Mn in cell culture media or during cell culture; or a combination of (i) and (ii) to modulate the Mn concentration. In certain embodiments, the leached Mn is produced by contact of the cell culture and/or cell culture media with: (i) a filter; (ii) a media preparation, hold, or culture vessel; or (iii) combinations of (i) and (ii). In certain embodiments, the filter includes but is not limited to: a depth filter, a column, a membrane and a disc. In certain embodiments, the filter material includes but is not limited to: diatomaceous earth, hollow fibers or a resin.

In certain embodiments, the cell culture medium is a basal medium, a reconstituted medium, a feed medium, a hydrolysate, a supplement, serum or an additive.

In certain embodiments, the cell culture medium is supplemented during the production stage of the cell culture.

In certain embodiments, the cell culture medium is supplemented prior to the production stage of the cell culture.

In certain embodiments, the cell culture medium comprises one or more of: Mn, fucose, galactose and/or Na+, and wherein the supplementation is based on a pre-defined schedule or criteria.

In certain embodiments, the one or more of the Mn, fucose, galactose and Na+ is supplemented as a bolus, as an intermittent supplement, as a continuous supplement, as a semi-continuous supplement, as a feedback loop-based supplement, or as a combination of one or more of thereof.

In certain embodiments, the cell culture medium consists essentially of one or more of: i) Mn; ii) fucose; iii) galactose; and/or iv) Na+.

In certain embodiments, the modulation of the Mn concentration comprises employing a cell culture media pH of about 6.1 to about 7.3; or about 6.3 to about 7.3 prior to High Temperature Short Time (HTST) heat treatment.

In certain embodiments, modulation of the glycosylation pattern of the glycoprotein of interest comprises modulating the pCO₂.

In certain embodiments, the cell culture or cell culture media is in a bioreactor and where modulation of pCO₂ is achieved by modulating: the bioreactor working volume; the bioreactor gas sparging strategy; the bioreactor agitation strategy; the bioreactor media exchange strategy, the bioreactor perfusion strategy, the bioreactor feed strategy, or an any combination thereof.

In certain embodiments, the pCO₂ is modulation comprises establishing a high pCO₂ culture. In certain embodiments, the pCO₂ is about 20 mmHg to about 250 mmHg; about 20 mmHg to about 250 mmHg; about 20 mmHg to about 150 mmHg; or about 30 mmHg to about 250 mmHg.

In certain embodiments, the pCO₂ is modulation comprises establishing a low pCO₂ culture. In certain embodiments, the pCO₂ is about 10 mmHg to about 100 mmHg; 10 mmHg to about 80 mmHg; about 20 mmHg to about 70 mmHg; or about 30 mmHg to about 60 mmHg.

In certain embodiments, the pCO₂ modulation occurs at day 0 of the culture.

In certain embodiments, the pCO₂ modulation occurs for: about the majority of the cell culture; about the first 5 days; about the first 7 days; or about the first 10 days.

In certain embodiments, the pCO₂ modulation occurs for: about the majority of the production culture; about the first 5 days; about the first 7 days; or about the first 10 days.

In certain embodiments, modulation of the glycosylation pattern of the glycoprotein of interest comprises modulating the duration of the pre-inoculation cell culture media hold, wherein the duration of the pre-inoculation cell culture media hold is about 0 hrs to about 120 hrs; about 0 hrs to about 72 hrs; about 0 hrs to about 48 hrs; or about 0 hrs to about 24 hrs.

In certain embodiments, the temperature of the media during the pre-inoculation cell culture media hold is about 25° C. to about 39° C.; about 30° C. to about 39° C.; about 35° C. to about 39° C.; or about 36° C. to about 39° C.

In certain embodiments, modulation of the glycosylation pattern of the glycoprotein of interest comprises modulating the duration of the cell culture, wherein the duration of the cell culture is about 0 days to about 150 days; about 0 days to about 15 days; about 0 days to about 12 days; 0 days to about 7 days; or about 0 days to about 5 days.

In certain embodiments, modulation of the glycosylation pattern of the glycoprotein of interest comprises modulating the Na+ concentration, wherein the Na+ concentration is about 0 mM to about 300 mM; is about 20 mM to about 20 mM; about 30 mM to about 150 mM; or about 40 mM to about 130 mM.

In certain embodiments, the modulation of the Na+ concentration comprises supplementing the cell culture with Na compounds including but not limited to: Na₂CO₃, NaHCO₃, NaOH, NaCl, or combinations thereof.

In certain embodiments, modulation of the glycosylation pattern of the glycoprotein of interest comprises modulating the osmolality, wherein the osmolality of is about 250 mOsm/kg to about 550 mOsm/kg; about 300 mOsm/kg to about 450 mOsm/kg; or about 325 mOsm/kg to about 425 mOsm/kg. In certain embodiments, the modulation of the osmolality comprises supplementing the cell culture with an osmolality-modulating media component. In certain embodiments, the osmolality-modulating media component is NaCl, KCl, sorbitol, an osmoprotectant, or combinations thereof. In certain embodiments, the osmolality-modulating media component is supplemented as a bolus, as an intermittent supplement, as a continuous supplement, as a semi-continuous supplement, as a feedback loop-based supplement, or as a combination of one or more of thereof.

In certain embodiments, modulation of the glycosylation pattern of the glycoprotein of interest comprises modulating the galactose concentration, wherein the galactose concentration is about 0 mM to about 60 mM or about 0 mM to about 50 mM.

In certain embodiments, modulation of the glycosylation pattern of the glycoprotein of interest comprises modulating the fucose concentration, wherein the fucose concentration is about 0 mM to about 60 mM; 0 mM to about 40 mM; about 0 mM to about 20 mM; or about 0 mM to about 10 mM.

In certain embodiments, modulation of the glycosylation pattern of the glycoprotein of interest comprises modulating the cell culture temperature, wherein the cell culture temperature is about 29° C. to about 39° C.; about 30° C. to about 39° C.; about 31° C. to about 38° C.; or about 34° C. to about 38° C.

In certain embodiments, the cell culture temperature is modulated during the production stage of the cell culture.

In certain embodiments, the cell culture temperature is modulated prior the production stage of the cell culture.

In certain embodiments, the cell culture temperature is modulated based on a pre-defined schedule or criteria.

In certain embodiments, the cell culture comprises eukaryotic cells. In certain embodiments, the eukaryotic cells are insect, avian, fungal, plant or mammalian cells. In certain embodiments, the fungal cells are yeast, Pichia or any filamentous fungal cells. In certain embodiments, the yeast cells are S. cerevisiae cells. In certain embodiments, the mammalian cells are CHO cells.

In certain embodiments, the cell culture is in a bioreactor including but not limited to: a single use technology (SUT) bag or bioreactor; a WAVE bioreactor; a stainless steel bioreactor; a flask; a tube and a chamber.

In certain embodiments, the volume of the cell culture is from 1 mL to 35,000 L. In certain embodiments, the volume of the cell culture is from 1 mL to 10 ml, from 1 mL to 50 ml, from 1 mL to 100 ml, from 1 mL to 200 ml, from 1 mL to 300 ml, from 1 mL to 500 ml, from 1 mL to 1000 ml, from 1 mL to 2000 ml, from 1 mL to 3000 ml, from 1 mL to 4000 ml, from 1 mL to 5000 ml, from 1 mL to 1 L, from 1 mL to 2 L, from 1 mL to 3 L, from 1 mL to 4 L, from 1 mL to 5 L, from 1 mL to 6 L, from 1 mL to 10 L, from 1 mL to 20 L, from 1 mL to 30 L, from 1 mL to 40 L, from 1 mL to 50 L, from 1 mL to 60 L, from 1 mL to 70 L, from 1 mL to 100 L, from 1 mL to 200 L, from 1 mL to 300 L, from 1 mL to 400 L, from 1 mL to 500 L, from 1 mL to 1000 L, from 1 mL to 2000 L, from 1 mL to 3000 L, from 1 mL to 4000 L, from 1 mL to 5000 L, from 1 mL to 10,000 L, from 1 mL to 20,000 L, from 1 mL to 30,000 L, from 1 mL to 30,000 L, from 1 mL to 35,000 L.

In certain embodiments, the present disclosure is directed to methods to prepare a cell culture media, a feed media, a hydrolysate, or an additive comprising one or more step(s) of modulating: the Mn concentration in a high partial pressure CO₂ (pCO₂) culture from about 1 nM to about 20000 nM; the Mn concentration in a low pCO₂ culture from about 1 nM to about 30000 nM; the pCO₂ from about 10 mmHg to about 250 mmHg; the pre-inoculation cell culture media hold duration from about 0 hrs to about 120 hrs; the cell culture duration from about 0 days to about 150 days; the Na+ concentration from about 0 mM to about 300 mM; the osmolality from about 250 mOsm/kg to about 550 mOsm/kg; the galactose concentration from about 0 mM to about 60 mM; the fucose concentration from about 0 mM to about 60 mM; and the cultivation temperature from about 29° C. to about 39° C.; wherein the cell culture media, feed media, hydrolysate, or additive modulates the glycosylation pattern of a glycoprotein of interest.

In certain embodiments, the methods involve modulating the pCO₂ from about 10 mmHg to about 250 mmHg, the Na+ concentration from about 0 mM to about 300 mM, and the duration of the pre-inoculation cell culture media hold from about 0 hrs to about 120 hrs.

In certain embodiments, the methods involve modulating the Mn concentration from about 1 nM to about 30000 nM, the pCO₂ from about 10 mmHg to about 250 mmHg, and the Na+ concentration from about 0 mM to about 300 mM.

In certain embodiments, the methods involve modulating the Mn concentration from about 1 nM to about 30000 nM, the pCO₂ from about 10 mmHg to about 250 mmHg, the Na+ concentration from about 0 mM to about 300 mM, and the duration of the pre-inoculation cell culture media hold from about 0 hrs to about 72 hrs.

In certain embodiments, the methods involve modulating the pCO₂ from about 10 mmHg to about 250 mmHg and the Na+ concentration from about 0 mM to about 300 mM.

In certain embodiments, the methods involve modulating the osmolality from about 250 mOsm/kg to about 550 mOsm/kg and the pCO₂ from about 10 mmHg to about 250 mmHg.

In certain embodiments, the methods involve modulating the pCO₂ from about 10 mmHg to about 250 mmHg, the Mn concentration from about 1 nM to about 30000 nM, the duration of the cell culture from about 0 days to about 150 days, and the duration of the pre-inoculation cell culture media hold from about 0 hrs to about 120 hrs

In certain embodiments, the methods involve modulating the Mn concentration from about 1 nM to about 30000 nM and the galactose concentration from about 0 mM to about 60 mM.

In certain embodiments, the methods involve modulating the fucose concentration from about 0 mM to about 60 mM and the Mn concentration from about 1 nM to about 30000 nM.

In certain embodiments, the methods involve modulating the fucose concentration from about 0 mM to about 60 mM and the pCO₂ from about 10 mmHg to about 250 mmHg.

In certain embodiments, the methods involve modulating the fucose concentration from about 0 mM to about 60 mM, the Mn concentration from about 1 nM to about 30000 nM, and the pCO₂ from about 10 mmHg to about 250 mmHg.

In certain embodiments, the methods involve modulating the fucose concentration from about 0 mM to about 60 mM and the cell culture temperature is about 29° C. to about 39° C.

In certain embodiments, the methods involve modulating the fucose concentration from about 0 mM to about 60 mM and the duration of the cell culture from about 0 days to about 150 days.

In certain embodiments, the Mn concentration is about 1 nM to about 20000 nM in a high pCO₂ culture; about 1 nM to about 1000 nM in a high pCO₂ culture; about 20 nM to about 300 nM in a high pCO₂ culture; or about 30 nM to about 110 nM in a high pCO₂ culture.

In certain embodiments, the Mn concentration is about 1 nM to about 30000 nM in a low pCO₂ culture; about 1 nM to about 3000 nM in a low pCO₂ culture; about 20 nM to about 300 nM in a low pCO₂ culture; or about 30 nM to about 110 nM in a low pCO₂ culture.

In certain embodiments, modulation of the Mn concentration comprises determining the Mn content in cell culture raw materials and selecting raw material lots to modulate the Mn concentration.

In certain embodiments, modulation of the Mn concentration comprises i) controlling materials that come into contract with cell culture media or cell culture; or (ii) accounting for the concentration of leached Mn in cell culture media or during cell culture; or a combination of (i) and (ii) to modulate the Mn concentration.

In certain embodiments, the leached Mn is produced by contact of the cell culture and/or cell culture media with: (i) a filter; (ii) a media preparation, hold, or culture vessel; or (iii) combinations of (i) and (ii). In certain embodiments, the filter includes but is not limited to: a depth filter, a column, a membrane and a disc. In certain embodiments, the filter material includes but is not limited to: diatomaceous earth, hollow fibers or a resin.

In certain embodiments, the modulation of the Mn concentration comprises employing a cell culture media pH of about 6.1 to about 7.3; or about 6.3 to about 7.3 prior to HTST treatment.

In certain embodiments, the pCO₂ is modulated. In certain embodiments, the cell culture media is in a bioreactor and where modulation of pCO₂ is achieved by modulating: the bioreactor working volume; the bioreactor gas sparging strategy; the bioreactor agitation strategy; the bioreactor feed strategy; the bioreactor perfusion strategy; the bioreactor media exchange strategy; or any combination thereof. In certain embodiments, the pCO₂ modulation comprises establishing a high pCO₂ culture. In certain embodiments, the pCO₂ is about 20 mmHg to about 250 mmHg; about 20 mmHg to about 250 mmHg; about 20 mmHg to about 150 mmHg; or about 30 mmHg to about 150 mmHg.

In certain embodiments, the pCO₂ is modulation comprises establishing a low pCO₂ culture. In certain embodiments, the pCO₂ is about 10 mmHg to about 100 mmHg; 10 mmHg to about 80 mmHg; about 20 mmHg to about 70 mmHg; or about 30 mmHg to about 60 mmHg. In certain embodiments, the pCO₂ modulation occurs at day 0 of the culture. In certain embodiments, the pCO₂ modulation occurs for: about the majority of the cell culture; about the first 5 days; about the first 7 days; or about the first 10 days. In certain embodiments, the pCO₂ modulation occurs for: about the majority of the production culture; about the first 5 days; about the first 7 days; or about the first 10 days.

In certain embodiments, the duration of the pre-inoculation cell culture media hold is about 0 hrs to about 120 hrs; 0 hrs to about 72 hrs; about 0 hrs to about 48 hrs; or about 0 hrs to about 24 hrs. In certain embodiments, the temperature of the media during the pre-inoculation cell culture media hold is about 25° C. to about 39° C.; about 30° C. to about 39° C.; about 35° C. to about 39° C.; or about 36° C. to about 39° C.

In certain embodiments, the duration of the cell culture is about 0 days to about 150 days; about 0 days to about 15 days; about 0 days to about 12 days; 0 days to about 7 days; or about 0 days to about 5 days.

In certain embodiments, the Na+ concentration is about 0 mM to about 300 mM; is about 20 mM to about 200 mM; about 30 mM to about 150 mM; or about 40 mM to about 130 mM. In certain embodiments, the modulation of the Na+ concentration comprises supplementing the cell culture with Na compounds including but not limited to: Na₂CO₃, NaHCO₃, NaOH, NaCl, or combinations thereof.

In certain embodiments, the osmolality of is about 250 mOsm/kg to about 550 mOsm/kg; about 300 mOsm/kg to about 450 mOsm/kg; or about 325 mOsm/kg to about 425 mOsm/kg. In certain embodiments, the modulation of the osmolality comprises supplementing the cell culture with an osmolality-modulating media component. In certain embodiments, the osmolality-modulating media component is NaCl, KCl, sorbitol, an osmoprotectant, or combinations thereof.

In certain embodiments, the galactose concentration is about 0 mM to about 60 mM or about 0 mM to about 50 mM.

In certain embodiments, the fucose concentration is about 0 mM to about 60 mM; 0 mM to about 40 mM; about 0 mM to about 20 mM; or about 0 mM to about 10 mM.

In certain embodiments, the cell culture temperature is about 29° C. to about 39° C.; about 30° C. to about 39° C.; about 31° C. to about 38° C.; or about 34° C. to about 38° C.

In certain embodiments, the present disclosure is directed to a eukaryotic cell fermentation process for the production of a recombinant protein. In certain embodiments, the recombinant protein is an antibody or antibody fragment, a scFv (single-chain variable fragment), BsDb (bispecific diabody), scBsDb (single-chain bispecific diabody), scBsTaFv (single-chain bispecific tandem variable domain), DNL-(Fab)3 (dock-and-lock trivalent Fab), sdAb (single-domain antibody) and BssdAb (bispecific single-domain antibody). In certain embodiments, the antibody is a chimeric, a humanized or a human antibody. In certain embodiments, the antibody is an anti-CD20 antibody. In certain embodiments, the anti-CD20 antibody is ocrelizumab. In certain embodiments, antibody or antibody fragment exhibits: a % G0-F (percent afucosylated glycoprotein) between about 0% to about 20%; about 1% to about 15%; about 1% to about 10%; or about 1% to about 8%; a normalized % G0-F between about 0% to about 20%; about 1% to about 15%; about 1% to about 10%; or about 1% to about 8%; and/or a % G0 (percent agalactosylated glycoprotein) between about 40% to about 90%; about 50% to about 90%; about 55% to about 85%; or about 60% to about 80%. In certain embodiments, the eukaryotic cell is an insect, avian, fungal, plant or mammalian cell. In certain embodiments, the fungal cells are yeast, Pichia or any filamentous fungal cells. In certain embodiments, the yeast cells are S. cerevisiae cells. In certain embodiments, the mammalian cells are CHO cells.

In certain embodiments, the present disclosure is directed to a cell culture composition comprising, a host cell engineered to express a glycoprotein of interest; and a cell culture and/or cell culture media modulated to target one or more predetermined parameter selected from: the Mn concentration in a high partial pressure CO₂ (pCO₂) culture from about 1 nM to about 20000 nM; the Mn concentration in a low pCO₂ culture from about 1 nM to about 30000 nM; the pCO₂ from about 10 mmHg to about 250 mmHg; the pre-inoculation cell culture media hold duration from about 0 hrs to about 120 hrs; the cell culture duration from about 0 days to about 150 days; the Na+ concentration from about 0 mM to about 300 mM; the osmolality from about 250 mOsm/kg to about 550 mOsm/kg; the galactose concentration from about 0 mM to about 60 mM; the fucose concentration from about 0 mM to about 60 mM; and the cultivation temperature from about 29° C. to about 39° C.

In certain embodiments, the Mn concentration is from about 1 nM to about 30000 nM and the duration of the pre-inoculation cell culture media hold is from about 0 hrs to about 120 hrs.

In certain embodiments, the pCO₂ is from about 10 mmHg to about 250 mmHg, the Na+ concentration from about 0 mM to about 300 mM, and the duration of the pre-inoculation cell culture media hold is from about 0 hrs to about 120 hrs.

In certain embodiments, the Mn concentration is from about 1 nM to about 30000 nM, the pCO₂ is from about 10 mmHg to about 250 mmHg, and the Na+ concentration is from about 0 mM to about 300 mM.

In certain embodiments, the Mn concentration is from about 1 nM to about 30000 nM, the pCO₂ is from about 10 mmHg to about 250 mmHg, the Na+ concentration is from about 0 mM to about 300 mM, and the duration of the pre-inoculation cell culture media hold is from about 0 hrs to about 120 hrs.

In certain embodiments, the pCO₂ is from about 10 mmHg to about 250 mmHg and the Na+ concentration is from about 0 mM to about 300 mM.

In certain embodiments, the osmolality is from about 250 mOsm/kg to about 550 mOsm/kg and the pCO₂ is from about 10 mmHg to about 250 mmHg.

In certain embodiments, the pCO₂ is from about 10 mmHg to about 250 mmHg, the Mn concentration is from about 1 nM to about 30000 nM, the duration of the cell culture is from about 0 days to about 150 days, and the duration of the pre-inoculation cell culture media hold is from about 0 hrs to about 120 hrs

In certain embodiments, the Mn concentration is from about 1 nM to about 30000 nM and the galactose concentration is from about 0 mM to about 60 mM. In certain embodiments, the fucose concentration is from about 0 mM to about 60 mM and the Mn concentration is from about 1 nM to about 30000 nM.

In certain embodiments, the fucose concentration is from about 0 mM to about 60 mM and the pCO₂ is from about 10 mmHg to about 250 mmHg. In certain embodiments, the fucose concentration is from about 0 mM to about 60 mM, the Mn concentration is from about 1 nM to about 30000 nM, and the pCO₂ is from about 10 mmHg to about 250 mmHg. In certain embodiments, the fucose concentration is from about 0 mM to about 60 mM and the cell culture temperature is about 29° C. to about 39° C.

In certain embodiments, the fucose concentration is from about 0 mM to about 60 mM and the duration of the cell culture is from about 0 days to about 150 days.

In certain embodiments, the present disclosure is directed to methods for producing a glycoprotein of interest in a cell culture, comprising: subjecting a cell culture medium suitable for cultivating a eukaryotic cell to the method according to any one of embodiments disclosed herein, inoculating the modulated cell culture medium with the eukaryotic cell that expresses the recombinant protein; cultivating the eukaryotic cell so that the recombinant protein is expressed.

In certain embodiments, the present disclosure is directed to methods of modulating the glycosylation of a glycoprotein of interest, the method comprising: assaying cell culture media to determine if the manganese concentration of the cell culture media falls within a targeted range; and culture a host cell engineered to express the glycoprotein of interest in the cell culture media falling within the targeted range; wherein the glycosylation of glycoproteins of interest is modulated as compared to the glycosylation of glycoproteins of interest expressed by the host cell in culture media falling outside the targeted range of manganese concentrations.

In certain embodiments, the present disclosure is directed to compositions comprising a glycoprotein of interest, wherein the preparation comprises: a cell culture media assayed to determine if the manganese concentration of the cell culture media falls within a targeted range; a host cell engineered to express a glycoprotein of interest; and the glycoprotein of interest.

In certain embodiments, the present disclosure is directed to methods of modulating the glycosylation of a glycoprotein of interest, the method comprising: supplementing a cell culture media employed in culturing host cells expressing the glycoprotein of interest with between about 10 nM and about 2000 nM manganese under high CO₂ conditions; or supplementing the cell culture supplementing the cell culture media employed in culturing a host cell expressing the glycoprotein of interest with between about 10 nM and bout 3000 nM manganese under low CO₂ conditions; wherein the glycosylation of glycoproteins of interest is modulated as compared to the glycosylation of glycoproteins of interest expressed by the host cell in culture media that has not been so supplemented.

In certain embodiments, the present disclosure is directed to cell culture compositions comprising, a cell culture media supplemented with: between about 10 nM and about 2000 nM manganese under high CO₂ conditions; or between about 10 nM and about 3000 nM manganese under low CO₂ conditions; and a host cell engineered to express a glycoprotein of interest.

In certain embodiments, the present disclosure is directed to compositions comprising a glycoprotein of interest, wherein the preparation comprises: a manganese supplemented cell culture media wherein the culture is supplemented with between about 10 nM and about 2000 nM manganese under high CO₂ conditions; or between about 10 nM and about 3000 nM manganese under low CO₂ conditions; a host cell engineered to express the glycoprotein of interest; and the glycoprotein of interest.

In certain embodiments, the method of modulating the glycosylation of a glycoprotein of interest comprises assaying cell culture media and/or cell cultures to determine if the manganese concentration of the cell culture media and/or cell cultures falls within a targeted range and culture a host cell engineered to express the glycoprotein of interest in the cell culture media falling within the targeted range. In certain embodiments, the glycosylation of glycoproteins of interest is modulated as compared to the glycosylation of glycoproteins of interest expressed by the host cell in culture media and/or cell cultures falling outside the targeted range of manganese concentrations. In some embodiments, the manganese concentration target range is between 20 nM and 200 nM. In non-limiting embodiments, the manganese concentration target range is between about 30 nM and about 110 nM.

In certain embodiments, the disclosed glycoprotein of interest is an antibody. The antibody can be a chimeric antibody, a humanized antibody, or a human antibody. In non-limiting embodiments, the antibody is ocrelizumab.

In certain embodiments, the disclosed host cell is a mammalian cell. The host cell can be a Chinese Hamster Ovary (CHO) cell.

In certain embodiments, the disclosed assaying of the cell culture media comprises assaying the manganese concentration of a component of the cell culture media. In certain embodiments, the disclosed assaying of the cell culture comprises assaying the manganese concentration of a component of the cell culture. The component of the cell culture media is a hydrolysate or a serum. The component of the cell culture media can also be a complex blend of multiple components.

In certain embodiments, the glycosylation is modulated to achieve an increased afucosylation (e.g., G0-F (afucosylated G0)), while decreasing agalactosylation (e.g., G0 (fucosylated, agalactosylated G0)). In certain embodiments, the glycosylation is modulated to achieve a decreased afucosylation (e.g., G0-F), while increasing agalactosylation (e.g., G0).

In certain embodiments, glycosylation is modulated to achieve an increased or decreased afucosylation (e.g., G0-F) without impacting agalactosylation (e.g., G0). In certain embodiments, glycosylation is modulated to achieve an increased or decreased agalactosylation (e.g., G0) without impacting afucosylation (e.g., G0-F).

In certain embodiments, the subject matter disclosed herein is directed to a cell culture composition comprising, a cell culture media and/or cell cultures assayed to determine if the manganese concentration of the cell culture media and/or cell cultures falls within a targeted range; and a host cell engineered to express a glycoprotein of interest. In certain embodiments, the manganese concentration is controlled through the selection or avoidance of raw materials that are in contact with culture media and/or cell cultures and can leach manganese (e.g., depth and/or media filters, media preparation and/or hold vessels, and bioreactors). In certain embodiments, the cell culture composition further comprises the glycoprotein of interest. In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell.

In certain embodiments, the subject matter disclosed herein is directed to a preparation comprising a glycoprotein of interest, wherein the preparation comprises a cell culture media assayed to determine if the manganese concentration of the cell culture media falls within a targeted range; a host cell engineered to express a glycoprotein of interest; and the glycoprotein of interest. In certain embodiments, the manganese concentration is controlled through the selection of raw materials that contain manganese at the desired levels. In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell.

In certain embodiments, the subject matter disclosed herein is directed to a method of modulating the glycosylation of a glycoprotein of interest, the method comprising supplementing a cell culture media employed in culturing host cells expressing the glycoprotein of interest with between about 10 nM and about 2000 nM manganese under high CO₂ conditions; or supplementing the cell culture supplementing the cell culture media employed in culturing a host cell expressing the glycoprotein of interest with between about 10 nM and about 3000 nM manganese under low CO₂ conditions; wherein the glycosylation of glycoproteins of interest is modulated as compared to the glycosylation of glycoproteins of interest expressed by the host cell in culture media that has not been so supplemented. In certain embodiments, the manganese is supplemented directly to the cell cultures. In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell. In certain embodiments, the glycosylation is modulated to achieve an increased afucosylation (e.g., G0-F (afucosylated G0)), while decreasing agalactosylation (e.g., G0 (fucosylated G0)). In certain embodiments, the glycosylation is modulated to achieve a decreased afucosylation (e.g., G0-F), while increasing agalactosylation (e.g., G0).

In certain embodiments, the subject matter disclosed herein is directed to a cell culture composition comprising, a cell culture media and/or cell culture supplemented with: between about 10 nM and about 2000 nM manganese under high CO₂ conditions; or between about 10 nM and about 3000 nM manganese under low CO₂ conditions; and a host cell engineered to express a glycoprotein of interest. In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell.

In certain embodiments, the subject matter disclosed herein is directed to a composition comprising a glycoprotein of interest, wherein the preparation comprises a manganese supplemented cell culture media wherein the culture is supplemented with between about 10 nM and about 2000 nM manganese under high CO₂ conditions; or between about 10 nM and about 3000 nM manganese under low CO₂ conditions; a host cell engineered to express the glycoprotein of interest; and the glycoprotein of interest. In certain embodiments, the manganese is supplemented by using raw materials that leach manganese during their contact with culture media and/or cell cultures (e.g., depth and/or media filters, media preparation and/or hold vessels, and bioreactors). In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell.

In certain embodiments, the subject matter disclosed herein is directed to a method of modulating the glycosylation of a glycoprotein of interest, the method comprising exposing cell culture media comprising a pH target of about 6.10 to about 7.25 to high temperature short time (HTST) heat treatment; and culturing a host cell expressing the glycoprotein of interest in the cell culture media; wherein the glycosylation of the glycoproteins of interest is modulated as compared to the glycosylation of the glycoproteins of interest expressed by the host cell in culture media where the pre-HTST heat treatment pH target is greater than pH 7.25. In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell. In certain embodiments, the glycosylation is modulated to achieve an increased G0-F (afucosylated G0), while decreasing G0 (fucosylated G0).

In certain embodiments, the subject matter disclosed herein is directed to a method of modulating the Mn level in the cell culture media and/or cell culture comprised of employing a cell culture media pH of about 6.1 to about 7.3 prior to High Temperature Short Time (HTST) heat treatment; and culturing a host cell expressing the glycoprotein of interest in the cell culture media. In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell. In certain embodiments, the glycosylation is modulated to achieve an increased G0-F (afucosylated G0), while decreasing G0 (fucosylated G0).

In certain embodiments, the subject matter disclosed herein is directed to a cell culture composition comprising, a cell culture media comprising a pH target of about 6.30 to about 7.25 exposed to a HTST heat treatment; and a host cell engineered to express a glycoprotein of interest. In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell.

In certain embodiments, the subject matter disclosed herein is directed to a cell culture composition comprising, a cell culture media comprising a pH target of about 6.3 to about 7.3 prior to exposure to a HTST heat treatment; and a host cell engineered to express a glycoprotein of interest. In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell.

In certain embodiments, the subject matter disclosed herein is directed to a composition comprising a glycoprotein of interest, wherein the preparation comprises a cell culture media comprising a pH target of about 6.10 to about 7.25 exposed to HTST heat treatment; a host cell engineered to express a glycoprotein of interest; and the glycoprotein of interest. In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell.

In certain embodiments, the subject matter disclosed herein is directed to a composition comprising a glycoprotein of interest, wherein the preparation comprises a cell culture media comprising a pH target of about 6.1 to about 7.3 prior to exposure to HTST heat treatment; a host cell engineered to express a glycoprotein of interest; and the glycoprotein of interest.

In certain embodiments, the subject matter disclosed herein is directed to a method of modulating the glycosylation of a glycoprotein of interest, the method comprising: culturing a host cell expressing the glycoprotein of interest in a cell culture media where the cell culture is supplemented with higher or lower levels of manganese, galactose, and/or fucose (or no supplementation), exposed to high or low pCO₂, the cell culture is exposed to an extended or shortened media hold time and/or culture duration, the culture is maintained at higher or lower cultivation temperature, maintained at higher or lower osmolality, and/or the cell culture comprises an increased or decreased Na+ concentration, and/or any combinations thereof; wherein the glycosylation of the glycoproteins of interest is modulated as compared to the fucosylation and/or galactosylation of a preparation of glycoproteins of interest expressed by the host cell in culture media exposed to low pCO₂, a shortened media hold time, and/or a reduced Na+ concentration. In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell. In certain embodiments, the glycosylation is modulated to achieve an in increased G0-F (afucosylated G0), while decreasing G0 (fucosylated G0) or a decreased G0-F (afucosylated G0), while increasing G0 (fucosylated G0). In certain embodiments, glycosylation is modulated to achieve an increased or decreased afucosylation (e.g., G0-F) without impacting agalactosylation (e.g., G0). In certain embodiments, glycosylation is modulated to achieve an increased or decreased agalactosylation (e.g., G0) without impacting afucosylation (e.g., G0-F).

In certain embodiments, the subject matter disclosed herein is directed to a cell culture composition comprising, a cell culture media and/or cell culture comprising manganese, galactose, and/or fucose supplementation (or no supplementation), high or low pCO₂, an extended or shortened media hold time, an extended or shortened culture duration, a higher or lower cultivation temperature, a higher or lower osmolality, and/or an increased or decreased Na+ concentration, and/or any combinations thereof; and a host cell engineered to express a glycoprotein of interest. In certain embodiments, the cell culture composition further comprises the glycoprotein of interest. In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell.

In certain embodiments, the subject matter disclosed herein is directed to a composition comprising a glycoprotein of interest, wherein the preparation comprises a cell culture media and/or cell cultures comprising manganese, galactose, and/or fucose supplementation (or no supplementation), high or low pCO₂, an extended or shortened media hold time, an extended or shortened culture duration, a higher or lower cultivation temperature, a higher or lower osmolality, and/or an increased or decreased Na+ concentration, and/or any combinations thereof; a host cell engineered to express a glycoprotein of interest; and the glycoprotein of interest. In certain embodiments, the glycoprotein of interest is an antibody. In certain embodiments, the antibody is a chimeric antibody, a humanized antibody, or a human antibody. In certain embodiments, the antibody is ocrelizumab. In certain embodiments, the host cell is a mammalian cell, e.g., a CHO cell.

### BRIEF DESCRIPTION OF THE DRAWINGS

FIG. 1 depicts variation in Mn levels on day 0 of production cultures correlate with variation in agalactosylation, % G0 (fucosylated G0, bottom) and afucosylation, normalized % G0-F (afucosylated, top) antibody species.

FIGS. 2A and 2B depict effects of Day 0 manganese concentration on galactosylation and fucosylation of mAb product in Ocrelizumab cell culture process. Plots of G0 against Day 0 Mn concentration (nM) are depicted in FIG. 2A. Plots of normalized G0-F against Day 0 Mn concentration (nM) are depicted in FIG. 2B.

FIGS. 3A and 3B depict effects of day 0 supplemental Mn on galactosylation and fucosylation of mAb product in Ocrelizumab cell culture process. Plots of G0 against supplemental Mn concentration (nM) are depicted in FIG. 3A. Plots G0-F against supplemental Mn concentration (nM) are depicted in FIG. 3B.

FIGS. 4A and 4B depict effects of day 0 Mn concentration on galactosylation and fucosylation of mAb product in Ocrelizumab cell culture process with various levels of cell culture scale. Plots of normalized G0-F against day 0 Mn concentration (nM) are depicted in FIG. 4A. Plots of G0 against day 0 Mn concentration (nM) are depicted in FIG. 4B. The 2 L scale-dependent factor refers to the use of high pCO₂ environment in the bioreactors.

FIGS. 5A and 5B depict effects of supplemental Mn on galactosylation and fucosylation of mAb product in Ocrelizumab cell culture process with various levels of cell culture scale. Plots of normalized G0-F against supplemental Mn concentration (nM) are depicted in FIG. 5A. Plots of G0 against supplemental Mn concentration (nM) are depicted in FIG. 5B. The 2 L scale-dependent factor refers to the use of high pCO₂ environment in the bioreactors.

FIGS. 6A and 6B depict effects of supplemental Mn on Antibody I cell culture process. Plots illustrating that Mn supplementation increases total afucosylation (G0) are depicted in FIG. 6A. Plots illustrating that Mn supplementation decreases agalactosylation (% GOF) are depicted in FIG. 6B.

FIG. 7 depicts effects of supplemental Mn on Antibody II cell culture process. Mn supplementation increases % G0-F (top) and decreases % G0 (bottom).

FIGS. 8A and 8B depict effects of supplemental Mn on Antibody III cell culture process. Plots illustrating that Mn supplementation increases % G0-F are depicted in FIG. 8A. Plots illustrating that Mn supplementation decreases % G0 are depicted in FIG. 8B.

FIGS. 9A and 9B depict effects of supplemental Mn on Antibody IV cell culture process. Plots illustrating that Mn supplementation increases % G0-F are depicted in FIG. 9A. Plots illustrating that Mn supplementation decreases % G0 are depicted in FIG. 9B.

FIGS. 10A and 10B depict effects of supplemental Mn on Antibody V cell culture process. Plots illustrating that Mn supplementation increases % G0-F are depicted in FIG. 10A. Plots illustrating that Mn supplementation decreases % G0 are depicted in FIG. 10B.

FIG. 11 depicts effects of Mn addition timing on glycosylation (top: % G0-F and bottom: % G0).

FIGS. 12A-12B depict effects of Mn addition timing during the production culture on glycosylation (FIG. 12A) and normalized G0-F (FIG. 12B).

FIG. 13 depicts exemplary typical and atypical High Temperature Short Time (HTST) pressures (top) and flow rate profiles (bottom) observed during Ocrelizumab HTST heat treatment.

FIG. 14 depicts turbidity changes (left) and Mn losses (right) versus pre-HTST pH adjustment of media in Sand Bath HTST screening.

FIG. 15 depicts impact of Pre-HTST pH Adjustment on 2 L Cell Culture Performance. Key Performance Indicators (% final viability (top), IVPCV (middle), and final titer (bottom)) are shown.

FIG. 16 depicts impact of Pre-HTST pH Adjustment on 2 L Cell Culture Performance. Charge-related variants (% light protected acidic region (top), % main IE-HPLC (middle), and % basic region (bottom)) are shown.

FIG. 17 depicts impact of Pre-HTST pH Adjustment on 2 L Cell Culture Performance. Size-related variants (% HMWS (top), main peak SE-HPLC (middle), and % Fab (bottom)) are shown.

FIG. 18 depicts impact of Pre-HTST pH Adjustment on glycans from a 2 L bioreactor. % G0, % G0-F, % normalized G0-F, % G2+ NANA, % Man5, % G1/G1′, % G2 are shown.

FIGS. 19A-19H depict effects of pH adjustment target for media prior to HTST heat treatment with Antibody III. A schematic diagram showing a design of experiment is depicted in FIG. 19A. A variability chart of manganese concentration for media pH targets prior to HTST heat treatment is depicted in FIG. 19B. A variability chart of final Viability (top) and IVPCV (bottom) is depicted in FIG. 19C. A variability chart of day 13 (left) and day 14 (right) titer is depicted in FIG. 19D. A variability chart of day 13 (left) and day 14% G0-F (right) is depicted in FIG. 19E. A variability chart of day 13 (left) and day 14% G0 (right) is depicted in FIG. 19F. A variability chart of day 13 (left column) and day 14 size variants (right column) is depicted in FIG. 19G, wherein the size-related variants include % BMWS, % main peak, and % LMWS. A variability chart of day 13 (left column) and day 14 charge-related variants (right column) is depicted in FIG. 19H, wherein the charge-related variants include % light protected acidic region, % main peak IE-HPLC, and % basic region.

FIGS. 20A and 20B depict schematic diagrams of exemplary bioreactors. A high partial pressure of carbon dioxide (pCO₂) model (top) and plots illustrating gassing strategies for maintaining constant dissolved oxygen in the high pCO₂ model (bottom) are depicted in FIG. 20A. A low pCO₂ model and plots illustrating gassing strategies for maintaining constant dissolved oxygen in the low pCO₂ model (bottom) are depicted in FIG. 20B.

FIGS. 21A and 21B depict effects of pCO₂ model, media hold, and Mn supplementation, and combinations thereof, on afucosylation (calculated as normalized G0-F) of mAb at time of harvest (day 12) and pCO₂ profiles for cultures. Plots illustrating that day 0 Mn levels are approximately five-fold higher in Mn-supplemented cultures compared to non-supplemented cultures are depicted in FIG. 21A. Plots illustrating pCO₂ profiles for cultures maintained in the low and high pCO₂ models are depicted in FIG. 21B.

FIGS. 22A-22D depict effects of pCO₂ and media hold on CHO cells. Plots illustrating afucosylation of mAb (calculated as normalized G0-F) at time of harvest (day 12) with increasing levels of Mn supplementation (at day 0) is depicted in FIG. 22A. Plots illustrating pCO₂ profiles during the cell culture are depicted in FIG. 22B. Plots illustrating osmolality profiles during the call culture are depicted in FIG. 22C. Plots illustrating Na⁺ profiles during the cell culture are depicted in FIG. 22D.

FIGS. 23A-23D depict effects of osmolality, pCO₂ model, and type of osmolality titrant on CHO cells. Plots illustrating afucosylation of mAb (calculated as normalized G0-F) at time of harvest (day 12) are depicted in FIG. 23A. Plots illustrating Na⁺ profiles during the cell culture are depicted in FIG. 23B. Plots illustrating pCO₂ profiles during the cell culture are depicted in FIG. 23C. Plots illustrating osmolality profiles during the call culture are depicted in FIG. 23D.

FIGS. 24A and 24B depict effects of pCO₂ and osmolality on intracellular pH (pH1) measured in CHO cells. Plots illustrating different pCO₂ levels while maintaining similar osmolality (406-413 mOsm/kg) and Na⁺ (83-87 mM) levels are depicted in FIG. 24A. Plots illustrating different osmolality levels (using NaCl as the osmolality titrant; 46-152 mM Na⁺) while maintaining similar pCO₂ levels (23-28 mm Hg) are depicted in FIG. 24B.

FIGS. 25A and 25B depict effects of different culture conditions and culture durations on afucosylation of mAb (calculated as normalized G0-F) produced in 3-L bioreactors. A chart showing differences in culture conditions is illustrated in FIG. 25A. Plots illustrating afucosylation levels on day 7 and at time of harvest (day 12) are depicted in FIG. 25B.

FIGS. 26A-26D depict a global proteome analysis. A schematic diagram showing an experimental design and a workflow is depicted in FIG. 26A. Plots illustrating principal component analysis (PCA) separated samples by day (PC1) and cell culture treatment (PC2) are depicted in FIG. 26B. Ingenuity pathway analysis (IPA) of canonical pathways for all cases is depicted in FIG. 26C. Expression of glycolytic enzymes for each treatment and day as compared to case is depicted in FIG. 26D.

FIGS. 27A-27C depict results of assessing the possibility that GDP-fucose is impacted in cell culture conditions that generated higher mAb afucosylation. De novo and salvage pathways for the synthesis of GDP-fucose are depicted in FIG. 27A. Heat map of key enzymes in the GDP-fucose synthesis pathways is depicted in FIG. 27B. Plots illustrating effects of L-fucose addition (on day 0) on afucosylation levels (calculated as normalized G0-F) at time of harvest (day 12) are depicted in FIG. 27C.

FIGS. 28A and 28B depict a proteomic analysis to determine differential expression of key proteins in the glycosylation pathway under different culture conditions in 3-L bioreactors. Description of the four cases (i-iv) tested in 3-L bioreactors and the resulting afucosylation levels are provided in FIG. 25A. A diagram of the glycosylation pathway in the Golgi illustrating only glycosylation variants relevant to afucosylation (Man5-G2) is depicted in FIG. 28A. Heat map of select glycosylation enzymes (FUT8, MAN I, GnT II, GalT3, GalT4, GalT7), intracellular and Golgi pH regulator (NHE1 and GPR89, respectively), and Mn level indicator proteins (ATP2A1, GPP13) on day 7 and day 12 of the production cultures is depicted in FIG. 28B.

FIGS. 29A-29E depict the performance of recombinant CHO cells cultured in 3-L bioreactors using high and low pCO₂ models. Growth represented by packed cell volume (PCV) is depicted in FIG. 29A. Plots representing viability of the recombinant CHO cells are depicted in FIG. 29B. Plots representing mAb titer are depicted in FIG. 29C. Plots representing charge variants (day 12) are depicted in FIG. 29D. Plots representing size variants (day 12) are depicted in FIG. 29E. HWMS refers to high molecular weight species; LWMS refers to low molecular weight species.

FIG. 30 depicts effects of pCO₂ model, media hold, and Mn supplementation on G0 of mAb at time of harvest (day 12). Plots show effects of each factor on its own, as well as in combination with other factors, on G0.

FIG. 31 depicts effects of L-fucose addition (on day 0) and manganese addition (on day 0) on G0 of mAb produced in 3-L bioreactors at the time of harvest (day 12). Plots show effects of fucose and manganese supplementation on their own, as well as their combined impact, on G0.

FIG. 32 depicts variability of manganese content in PP3 and GEM.

FIGS. 33A-33D depict effects of media hold, Mn supplementation, and a combination thereof on G0 and G0-F. FIGS. 33A-33B depict effects of media hold time at elevated temperature (38° C.) on agalactosylation, G0 (FIG. 34A) and afucosylation, normalized G0-F (FIG. 33B). FIG. 33C depicts cumulative effects of media hold on afucosylation (% G0-F) for Antibody III. FIG. 33D depicts effects of media hold, Mn supplementation, and a combination thereof, on afucosylation (% G0-F) for Antibody III.

FIGS. 34A-34B depict effects of galactose and supplemental Mn, and their interactions, on agalactosylation, G0 (FIG. 34A) and afucosylation, normalized G0-F (FIG. 34B) from Study 1.

FIGS. 35A-35B depict effects of galactose and Mn, and their interactions, on agalactosylation, G0 (FIG. 35A) and afucosylation, normalized G0-F (FIG. 35B) from Study 2.

FIGS. 36A-36B depict effects of galactose on agalactosylation, G0 (FIG. 36A) and afucosylation, normalized G0-F (FIG. 36B) from Study 3.

FIGS. 37A-37B depict effects of fucose supplementation on afucosylation, G0-F (FIG. 37A) and agalactosylation, G0 (FIG. 37B).

FIGS. 38A-38B depict effects of fucose addition timing on afucosylation, G0-F (FIG. 38A) and agalactosylation, G0 (FIG. 38B).

FIGS. 39A-39B depict effects of fucose concentration and temperature, and their interactions, on afucosylation, G0-F (FIG. 39A) and agalactosylation, G0 (FIG. 39B).

### DETAILED DESCRIPTION

The subject matter disclosed herein relates to modulating the glycosylation (e.g., galactosylation and/or fucosylation) of a recombinant glycoprotein of interest, e.g., a mAb, such that it falls within desirable quality attribute ranges. For example, but not by way of limitation, the subject matter disclosed herein is applicable to modifying the glycosylation profile of a mAb to fall within a narrower band of quality attribute ranges than achieved using conventional cell culture media, media preparation strategies, and/or cell culture strategies. Methods by which glycosylation can be modulated in accordance with the instant disclosure include, but are not limited to: (1) control of cell culture media manganese (Mn) concentration, e.g., with respect to Mn concentration analysis of raw materials, Mn supplementation during cell culture, and/or establishing a reduced pH set point for media pH adjustment prior to High Temperature Short Time (HTST) heat treatment of the media; and (2) controlling process parameters during cell culture, e.g., pCO₂, media hold duration, and osmolality/Na⁺. The subject matter of the present disclosure is also directed to cell culture and glycoprotein compositions prepared when such process parameters are controlled as described herein.

For purposes of clarity of disclosure and not by way of limitation, the detailed description is divided into the following subsections:

1. Definitions

2. Control of Raw Materials to Modulate Glycosylation

3. Manganese Supplementation to Modulate Glycosylation

4. Modified pH Target of Media Prior to High Temperature Short Time (HTST) Treatment to Modulate Glycosylation by Minimizing Manganese Loss during HTST

5. pCO₂, Manganese, Media Hold, Culture Duration, Cultivation Temperature, and Osmolality/Na⁺ to Modulate Glycosylation

6. Galactose to Modulate Glycosylation

7. Fucose and Cultivation Temperature, and their Combination to Modulate Glycosylation

8. Cell Culture and Glycoprotein Compositions

### 1. Definitions

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in practice or testing of the presently disclosed subject matter. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of”, and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “supplementation” is used in the broadest sense and encompass various types, techniques, or methods for adding target molecules, materials, objects, or combinations thereof. Bolus, fully continuous, semi-continuous, intermittent, time-based, feedback-loop based additions are examples of the supplementation.

The term “modulate” is used herein to refer to an increase or decrease in the respective attribute.

The term “antibody” herein is used in the broadest sense and encompasses various antibody structures, including but not limited to monoclonal antibodies, polyclonal antibodies, multispecific antibodies (e.g., bispecific antibodies), half antibodies, and antibody fragments so long as they exhibit a desired antigen-binding activity.

As used herein, the term “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments.

As used herein, the term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind to a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

As used herein, the term “homologous sequences” refers to sequences that share a significant sequence similarity as determined by an alignment of the sequences. For example, two sequences can be about 50%, about 60%, about 70%, about 80%, about 90%, about 95%, about 99%, or about 99.9% homologous. The alignment is carried out by algorithms and computer programs including, but not limited to, BLAST, FASTA, and HMME, which compares sequences and calculates the statistical significance of matches based on factors such as sequence length, sequence identify and similarity, and the presence and length of sequence mismatches and gaps. Homologous sequences can refer to both DNA and protein sequences

The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

The term “glycoprotein” refers to a polypeptide or protein coupled to at least one carbohydrate moiety, e.g., a polysaccharide or an oligosaccharide, that is attached to the protein via an oxygen-containing or a nitrogen-containing side chain of an amino acid residue, e.g., a serine or threonine residue (“O-linked”) or an asparagine residue (“N-linked”). The term “glycan” refers to a polysaccharide or an oligosaccharide, e.g., a polymer comprised of monosaccharides. Glycans can be homo- or heteropolymers of monosaccharide residues and can be linear or branched.

As used herein, the “glycosylation pattern” and “glycosylation profile” of a recombinant glycoprotein of interest refers to various physical characteristics of the glycoprotein's polysaccharides or oligosaccharides, such as, e.g., the quantity and quality of various monosaccharides present, the degree of branching, and/or the attachment (e.g., N-linked or O-linked).

“Fucosylation” refers to the degree and distribution of fucose residues on polysaccharides and oligosaccharides, for example, N-glycans, 0-glycans and glycolipids. “Afucosylation” refers to the lack of fucose residues on polysaccharides and oligosaccharides. G0 glycans refers to glycans that lack terminal galactose residues. The art has identified two distinct nomenclatures for identifying fucosylated/afucosylated G0 glycans:
(1) where “G0-F” (i.e., “G0 ‘minus’ F”) is employed to reference afucosylated G0 glycans, then “G0” is employed to reference fucosylated G0 glycans; and
(2) where “G0” is employed to reference afucosylated G0 glycans, then “GOF” is employed to reference fucosylated G0 glycans.
   Identification of which convention is being used in a specific context involves analyzing the use of G0 and the use of either G0-F or GOF. Therapeutic glycoproteins, e.g., antibodies or Fc fusion proteins, with non-fucosylated, or “afucosylated” N-glycans exhibit enhanced antibody-dependent cellular cytotoxicity (ADCC) due to the enhancement of FcγRIIIa binding capacity without any detectable change in complement-dependent cytotoxicity (CDC) or antigen binding capability. In certain situations, e.g., cancer treatment, non-fucosylated or “afucosylated” antibodies are desirable because they can achieve therapeutic efficacy at low doses, while inducing high cellular cytotoxicity against tumor cells, and triggering high effector function in NK cells via enhanced interaction with FcγRIIIa. In other situations, e.g., treatment of inflammatory or autoimmune diseases, enhanced ADCC and FcγRIIIa binding is not desirable, and accordingly therapeutic glycoproteins with higher levels of fucose residues in their N-glycans can be preferable. As used herein, the term “% afucose” or “% afucosylation” refers to the percentage of non-fucosylated N-glycans present on a recombinant glycoprotein of interest. A higher % afucose or % afucosylation denotes a higher number of non-fucosylated N-glycans, and a lower % afucose or % afucosylation denotes a higher number of fucosylated N-glycans. Afucosylation can sometimes be represented as % normalized G0-F, which is calculated by:

Normalized G0−F(%)=G0−F(%)/G0(%)+G0−F(%)×100%

The term “galactosylation” as used within the context of the present invention refers to addition of a galactose unit to an oligosaccharide chain on a glycoprotein. The term “agalactosylation” refers to the lack of galactose unit on an oligosaccharide chain on a glycoprotein. The term “galactosylated” antibody as used herein refers to an antibody, wherein the N-linked glycan of the antibody comprises at least one galactose residue (e.g., G1 and G2 glycans). The term “agalactosylated” antibody as used herein refers to an antibody, wherein the N-linked glycan of the antibody is devoid of a galactose residue (e.g., G0 and G0F glycans).

As used herein, the term “expression” refers to transcription and/or translation. In certain embodiments, the level of transcription of a desired product can be determined based on the amount of corresponding mRNA that is present. For example, mRNA transcribed from a sequence of interest can be quantitated by PCR or by Northern hybridization. In certain embodiments, protein encoded by a sequence of interest can be quantitated by various methods, e.g. by ELISA, by assaying for the biological activity of the protein, or by employing assays that are independent of such activity, such as Western blotting or radioimmunoassay, using antibodies that recognize and bind to the protein

As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. In certain embodiments, vectors direct the expression of nucleic acids to which they are operatively linked. Such vectors are referred to herein as “expression vectors.”

“Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII and FcγRIII FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and Natural Killer (NK) cells. Alternatively, or additionally, ADCC activity of a molecule of interest may be assessed in vivo, e.g., in an animal model such as that disclosed in Clynes et al. PNAS (USA) 95:652-656 (1998).

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased Clq binding capability are described, e.g., in U.S. Pat. No. 6,194,551 B1 and WO 1999/51642. See also, e.g., Idusogie et al. J. Immunol. 164: 4178-4184 (2000).

“Culturing” a cell refers to contacting a cell with a cell culture medium under conditions suitable to the survival and/or growth of the cell and/or proliferation of the cell. Cell culture can be performed under a variety of conditions including but not limited to batch, fed-batch, continuous, perfusion processes. Cell culture duration may vary depending the process. For example, but not by way of limitation, a fed-batch process can be run for a fewer number of days, for e.g., from 0 to 20 days, whereas a typical perfusion process can run up to 150 days or even more days.

“Batch culture” refers to a culture in which all components for cell culturing (including the cells and all culture nutrients) are supplied to the culturing vessel at the start of the culturing process.

The phrase “fed batch cell culture,” as used herein refers to a batch culture wherein the cells and culture medium are supplied to the culturing vessel initially, and additional culture nutrients are fed, continuously or in discrete increments, to the culture during the culturing process, with or without periodic cell and/or product harvest before termination of culture.

The phrase “bioreactor agitation strategy” refers to the agitation rate and/or physical manipulation of the culture and/or culture media in the bioreactor.

The phrase “bioreactor media exchange strategy” refers to any process by which a change in media contacting the bioreactor and/or the cells of the culture occurs, including, but not limited to, processes where cells are spun down from a cell culture sample taken from a bioreactor and resuspended in a new medium which may be different from the original cell culture medium used to grow the cells initially.

“Perfusion culture” is a culture by which the cells are restrained in the culture by, e.g., filtration, encapsulation, anchoring to microcarriers, etc., and the culture medium is continuously or intermittently introduced and removed from the culturing vessel.

“Culturing vessel”, “culture vessel”, and “bioreactor” refer to a container used for culturing cells. The culturing vessel can be of any size so long as it is useful for the culturing of cells. In certain embodiments, the bioreactors for use in the presently disclosed methods are stainless steel vessels. In certain embodiments, the bioreactors for use in the presently disclosed methods are rocker bags. In certain embodiments, the bioreactors for use in the presently disclosed methods are single-use bioreactors.

The terms “medium” and “cell culture medium” refer to a nutrient source used for growing or maintaining cells. As is understood by a person of skill in the art, the nutrient source may contain components required by the cell for growth and/or survival or may contain components that aid in cell growth and/or survival. Cell culture medium also refers to any fluid supernatants for growing or maintaining cells. Medium components refer to any components which can be added to the cell culture or the cell culture medium at any culture stage, at any time, or in any form. Medium components also refer to components from the raw materials for the cell culture medium. Vitamins, essential or non-essential amino acids, and trace elements are examples of medium components. It is to be understood that “medium” and “media” are used interchangeably throughout this specification.

A “chemically defined cell culture medium” or “CDM” is a medium with a specified composition that is free of animal-derived or undefined products such as animal serum and peptone. As would be understood by a person of skill in the art, a CDM may be used in a process of polypeptide production whereby a cell is in contact with, and secretes a polypeptide into, the CDM. Thus, it is understood that a composition may contain a CDM and a polypeptide product and that the presence of the polypeptide product does not render the CDM chemically undefined.

A “chemically undefined cell culture medium” refers to a medium whose chemical composition cannot be specified, and which may contain one or more animal-derived or undefined products such as animal serum and peptone. As would be understood by a person of skill in the art, a chemically undefined cell culture medium may contain an animal-derived product as a nutrient source.

“Media hold” refers to the cell culture practice of holding cell culture media in culture vessels (e.g., bioreactors, single-use bags) or vessels used for media preparation or media storage (e.g., stainless steel tanks, single-use containers) prior to use in culturing cells. In cell culture operations, culture media can be warmed and then held at or close to the cultivation temperature before using the media to inoculate cells in a bioreactor. “Media hold duration” or “media hold time” refers to the extent of time that the media is held (e.g., at temperature above ambient) before it is used to inoculate cells in a bioreactor. It is understood that “media hold”, “media hold duration”, and “media hold time” are used interchangeably throughout this specification.

“HTST” refers to “high-temperature short-time” treatment of cell culture media. This HTST treatment of cell culture media can provide an additional safety barrier against adventitious agents. (Floris et al., (2018) Appl Microbiol Biotechnol. 102(13):5495-5504; Pohlscheidt et al., (2014) Appl Microbiol Biotechnol. 98(7):2965-71. During HTST treatment of cell culture media, precipitates may form, HTST equipment may foul, and media components may fall out of solution. Adjustments of specific culture media parameters may be performed for lowering or preventing formation of precipitates in the media from HTST treatment.

As used herein, the phrase “low pCO₂” describes operations in a relatively narrow carbon dioxide range, with the upper limit of CO2 being lower than that used in a “high pCO₂” operation. Low pCO₂ can be from about 10 mmHg to about 100 mmHg, about 10 mm Hg to about 80 mmHg, about 10 mmHg to about 70 mmHg, or about 10 mmHg to about 60 mmHg. “High pCO2”, in contrast, is used herein to refer to in a broader carbon dioxide range, with the upper limit of pCO₂ being higher than that used in a low pCO₂ operation. High pCO₂ can be from about 20 to about 250 mmHg, about 20 mmHg to about 200 mmHg, about 20 mmHg to about 150 mmHg, or about 30 mmHg to 150 mmHg. The pCO₂ modulation described herein can occur for at least the first half of cell culture duration. For example, but not by way of limitation, for a 20-day culture, pCO₂ modulation can take place for at least the first 10 days. Depending on varied cell culture durations, the pCO₂ modulation will also vary accordingly.

2. Control of Raw Materials to Modulate Glycosylation

Multiple cell culture factors are known to have the potential to impact glycosylation of glycoproteins, e.g., mAbs. These factors include process parameters and media components, such as galactose and trace metals, among others. Variation in levels of individual media components can be introduced into mAb cell culture process via the use of complex raw materials. For example, but not by way of limitation, cell culture media, e.g., basal media or feed media (as well as individual components thereof, e.g., hydrolysates or various types of serum), can exhibit lot-to-lot variation that can impact mAb glycosylation. Accordingly, in certain embodiments, the present disclosure is directed to compositions and methods aimed at reducing cell culture media variability to modulate mAb glycosylation (e.g., galactosylation and/or fucosylation). For example, but not by way of limitation, Mn supplementation can be achieved by using media components that contain Mn as impurities or raw materials that can release Mn to the cell culture media or cell cultures (e.g., depth filters, stainless steel or glass vessels).

In certain embodiments, the present disclosure is directed to strategies for screening cell culture media and/or individual components thereof in order to modulate mAb glycosylation (e.g., galactosylation and/or fucosylation). In non-limiting embodiments, cell culture media compliance with specific target amounts of individual components, e.g., Mn concentration, galactose concentration, can be screened. For example, but not by way of limitation, cell culture media can be screened and selected based on Mn concentration target range of about 1 nM to about 10 μM, about 1 nM to about 1 μM, about 20 nM to about 300 nM, or about 30 nM to about 110 nM (where media falling outside of such a target range is not employed in connection with cell culture of the mAb). In certain embodiments, cell culture media can be further supplemented with galactose up to 10 g/L (e.g., about 0 g/L, about 2 g/L, about 3 g/L, about 4 g/L, about 7 g/L, or about 10 g/L). In certain embodiments, cell culture media can be supplemented with galactose up to about 6 g/L.

Certain embodiments described herein relate to modulating glycosylation (e.g., afucosylation and/or galactosylation) by screening cell culture media and/or cell cultures based on the disclosed Mn concentration target ranges to achieve or preserve a desired glycoprotein glycosylation pattern. In certain non-limiting embodiments, the desired glycoprotein glycosylation pattern can be a modulation in afucosylation and/or galactosylation of the glycoprotein. For example, but not by way of limitation, a target range of afucosylated G0 (% G0-F or % normalized G0-F) can be between about 0% to about 20%, about 1% to about 15%, about 1% to about 10%, or about 1% to about 8%. The % G0-F or % normalized G0-F can be modulated by about 0.5%, 1%, about 2%, about 3%, about 4%, about 5%, about 6%, about 7%, about 8%, about 9%, about 10%, about 11%, about 12%, about 13%, about 14%, about 15%, or about 20%. In certain embodiments, the desired glycoprotein glycosylation pattern can be a decrease or an increase in fucosylation of the glycoprotein. For example, but not by way of limitation, a target range of fucosylated, agalactosylated G0 (% G0) can be between about 40% to about 90%, about 50% to about 90%, about 50% to about 85%, or about 60% to about 80%. The % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%.

In certain embodiments, the desired glycoprotein glycosylation pattern can be a combination of modulation in afucosylation and galactosylation of the glycoprotein. For example, but not by way of limitation, a target range of % G0-F or % normalized G0-F can be between about 0% to about 20%, about 1% to about 15%, about 1% to about 10%, or about 1% to about 8%, and a target range of % G0 can be between about 40% to about 90%, about 50% to about 90%, about 55% to about 85%, or about 60% to about 80%. The % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%, and the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%.

3. Manganese Supplementation to Modulate Glycosylation

As described herein, cell culture media Mn concentration can impact glycosylation, e.g., mAb galactosylation and/or fucosylation. As also described herein, cell culture Mn concentration can impact glycosylation, e.g., mAb galactosylation and/or fucosylation. Accordingly, in certain embodiments, mAb glycosylation can be modulated not only by controlling for the amount of Mn present in cell culture media raw materials, as described above, but also by supplementing cell culture media with Mn. In certain embodiments, an increase in Mn concentration can increase afucosylation (by increasing levels of G0-F, the afucosylated form of G0), and/or increase galactosylation (which results in decreasing G0, the agalactosylated and fucosylated glycan species).

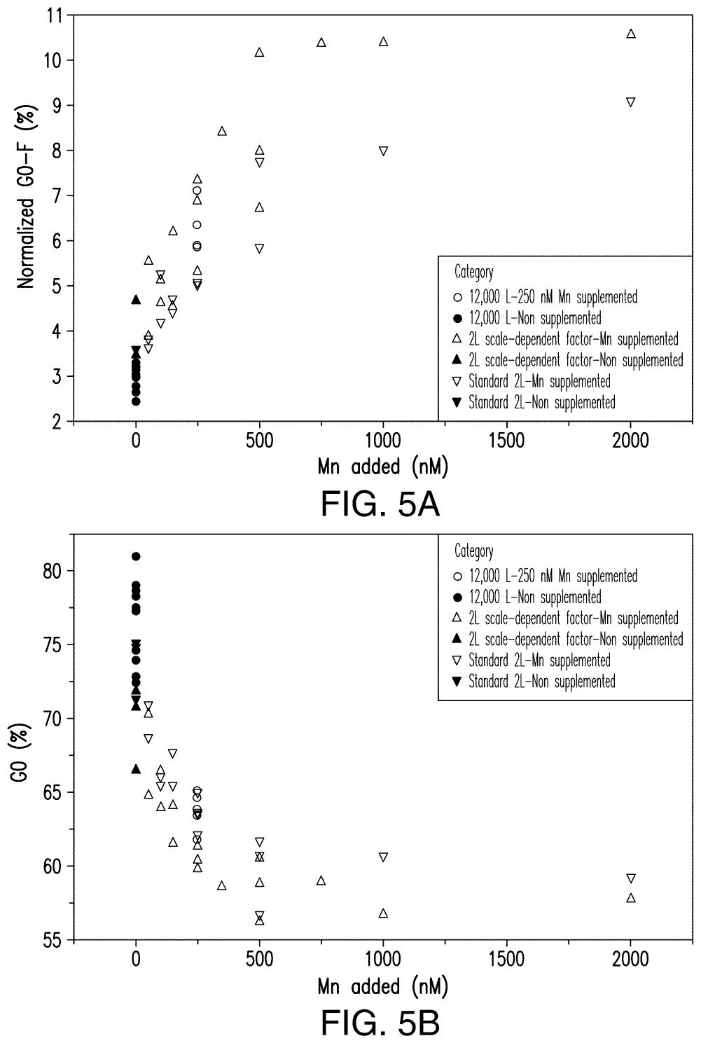

While increases in Mn concentration in cell culture media and/or cell culture can generally result in increased G0-F (afucosylated G0) and decreased G0 (fucosylated G0) as outlined in FIGS. 5-10, identifying that trend across six distinct mAbs, the extent to which afucosylation (and hence the G0-F species) increases and agalactosylation (and hence the G0 species) decreases can be refined by establishing specific Mn supplementation action targets. For example, in certain embodiments, the concentration of Mn to be supplemented should be sufficient to increase the G0-F and decrease the G0, while not rendering the resulting mAb out of desired product quality specifications. In certain embodiments, the Mn is supplemented to achieve the selected range in cell culture media and/or cell cultures. In certain embodiments, the concentration of Mn supplementation is selected to be less than about 10 μM (e.g., about 10 nM, about 40 nM, about 100 nM, about 150 nM, about 200 nM, about 250 nM, about 500 nM, about 700 nM, about 750 nM, about 1000 nM, about 1500 nM, about 2000 nM, about 3000 nM, about 5000 nM, about 8000 nM, or about 10 μM, including concentrations falling within the ranges disclosed). In certain embodiments, the concentration of Mn supplementation can be between about 20 nM and about 300 nM. In non-limiting embodiments, the concentration of Mn supplementation can be between about 30 nM and about 110 nM. In certain embodiments, including those where the Mn supplementation is occurring in a cell culture media exposed to high CO₂, the concentration of Mn supplementation is selected to be less than 3000 nM (e.g., about 5 nM, 10 nM, about 30 nM, 40 nM, about 50 nM, 100 nM, about 200 nM, about 250 nM, about 500 nM, about 1000 nM, about 2000 nM, about 3000 nM, including concentrations falling within the ranges disclosed). As outlined herein, such concentrations have the unexpected ability to increase afucosylation (and hence G0-F glycans) and decrease agalactosylation (and hence G0 glycans), while not rendering the resulting mAb out of desired product quality specifications.

In certain embodiments, the timing of Mn supplementation to the culture can also impact glycosylation (e.g., galactosylation and afucosylation). Mn supplementation can be added during the expansion culture stages prior to production and/or during the production culture stage. In certain embodiments, Mn supplementation can occur from the leaching of Mn from materials in contact with cell culture media and/or cell cultures (e.g., depth or media filters, culture vessels, media hold vessels). In non-limiting embodiments, Mn supplementation can be achieved by using depth filters containing diatomaceous earth, which leaches Mn and other trace metals, during the media preparation filtration process, thereby supplementing the culture.

In certain embodiments, mAb can be harvested after Mn supplementation. For example, but not by way of limitation, mAb can be harvested between about day 2 of the culture and about day 25 of the culture (e.g., at about day 2, 3, 4, 5, 6, 7, 8, 9, 10, 11, 12, 13, 14, 15, 16, 17, 18, 19, 20, 21, 22, 23, 24, or 25 of the cell culture. In non-limiting embodiments, mAb can be harvested between about day 7 and about day 15 of the cell culture. In some embodiments, mAb can be harvested between about day 5 and about day 20 of the cell culture).

In certain embodiments, the media compositions and cell culture processes disclosed herein can be combined with additional and/or alternative glycosylation-modulating concentrations of one or more of a group consisting of the following: fucose, ammonia, sodium, uridine, N-acetylglucosamine, N-acetylgalactosamine, cadmium, lipoic acid, divalent metal ions such as V²⁺, Cr²⁺, Fe²⁺, Co²⁺, Ni²⁺, Cu²⁺, Zn²⁺, Ca²⁺, Mg²⁺, and kifunensine.

4. Modified pH Target of Media Prior to High Temperature Short Time (HTST) Treatment to Modulate Glycosylation

As disclosed herein, cell culture media concentration of Mn can modulate glycoprotein, e.g., mAb, glycosylation. Accordingly, the present disclosure is directed, in certain embodiments, to methods of controlling cell culture media Mn concentration in order to modulate glycosylation, e.g., galactosylation and/or fucosylation of mAbs. The present disclosure is also directed, in certain embodiments, to methods of controlling cell culture Mn concentration in order to modulate glycosylation, e.g., galactosylation and/or fucosylation of mAbs. For example, but not by way of limitation, the present disclosure notes that performing HTST treatment of media with a pre-HTST media pH adjustment target of greater than about 7.0 can result in a decrease in cell culture media Mn concentration after HTST treatment. Thus, in certain embodiments, the present disclosure is directed to performing HTST with media prepared to a pH target of less than about 7.25 (e.g., between about 6.1 and about 7.2). In certain embodiments, the present disclosure is directed to performing HTST with media prepared to a pH target of less than about 7.3 (e.g., between about 6.1 and about 7.3). In certain embodiments, the pH target for the media prepared for HTST treatment can be about 6.1, about 6.3, about 6.4, about 6.5, about 6.6, about 6.7, about 6.8, about 6.9, about 7.1, about 7.2, or about 7.3.

5. pCO2, Manganese, Media Hold, Culture Duration, Cultivation Temperature, and Na+/Osmolality to Modulate Glycosylation

As disclosed herein, controlling the pCO₂, media hold duration, culture duration, cultivation temperature, manganese concentration, osmolality/Na+ concentration, and/or a combination thereof, of a cell culture media and/or cell cultures can result in modulation of the fucosylated and/or afucosylated G0 glycans of a glycoprotein, e.g., a mAb, cultured in such media. In certain embodiments, the present disclosure is directed to methods of cell culture employing media or cell cultures where the pCO₂, manganese concentration, media hold duration, culture duration, cultivation temperature, Na+ concentration, osmolality, or a combination thereof, have been controlled as outlined herein.

5.1 Media Hold

As described herein, media hold duration at a particular temperature, or temperature range, can impact glycosylation (e.g., galactosylation and/or afucosylation). In certain embodiments, elevated media hold temperature can be between about 25° C. and about 39° C., about 30° C. to about 39° C., about 35° C. to about 39° C., or about 36° C. to about 39° C. In certain embodiments, the media hold duration at a particular temperature, or temperature range, ranges from about 0 hours to about 12 hours, about 0 hours to about 24 hours, about 0 hours to about 36 hours, about 0 hours to about 48 hours, about 0 hours to about 60 hours, about 0 hours to about 72 hours, about 0 hours to about 96 hours, or more. In certain non-limiting embodiments, cell culture media is held at the temperature between about 25° C. and about 39° C. for a period of about 0 hours to about 72 hours to modulate glycosylation (e.g., afucosylation and/or galactosylation). In certain embodiments, the cell culture media held in this manner is employed in a production culture, an expansion culture, or both.

Certain embodiments described herein relate to modulating glycosylation (e.g., afucosylation and/or galactosylation), by applying the disclosed media hold time at a particular temperature, or temperature range, to achieve or preserve a desired glycoprotein glycosylation pattern. In certain embodiments, the desired glycoprotein glycosylation pattern can be a modulation in afucosylation of the glycoprotein. For example, but not by way of limitation, a target range of afucosylated G0 (% G0-F or % normalized G0-F) can be between about 0% to about 20%, about 1% to about 15%, about 1% to about 10%, or about 1% to about 8%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%. In certain embodiments, the desired glycoprotein glycosylation pattern can be a modulation in galactosylation of the glycoprotein. For example, but not by way of limitation, a target range of agalactosylation (e.g., % G0) can be between about 40% to about 90%, about 50% to about 90%, about 55% to about 85%, or about 60% to about 80%. In certain embodiments, the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%. In certain embodiments, the desired glycoprotein glycosylation pattern can be a combination of modulation in afucosylation and galactosylation of the glycoprotein. For example, but not by way of limitation, a target range of % G0-F or % normalized G0-F can be between about 0% to about 20%, about 1% to about 15%, about 1% to about 10%, or about 1% to about 8% and a target range of % G0 can be between about 40% to about 90%, about 50% to about 90%, about 55% to about 85%, or about 60% to about 80%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%, and the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%.

5.2 Partial Pressure of Carbon Dioxide (pCO₂)

In certain embodiments, the present disclosure is directed to strategies for adjusting partial pressure of carbon dioxide (pCO₂) in cell cultures to modulate mAb glycosylation (e.g., galactosylation and/or fucosylation). In non-limiting embodiments, level of pCO₂ can be between 0 mm Hg to 250 mm Hg. High pCO₂ model can have pCO₂ range of about 0 mmHg to about 250 mmHg, about 20 mmHg to about 250 mmHg, about 20 mmHg to about 200 mmHg, about 20 mmHg to about 150 mmHg, or about 30 mmHg to 150 mmHg for the majority of the culture duration starting from day 0. Low pCO₂ model can have pCO₂ range of about 10 mm Hg to about 100 mmHg, about 10 mm Hg to about 80 mmHg, about 10 mm Hg to about 70 mmHg, or about 10 mm Hg to about 60 mmHg for the majority of the culture duration starting from day 0. Certain embodiments described herein relate to modulating glycosylation by adjusting level of pCO₂ to the target ranges to achieve or preserve a desired glycoprotein glycosylation pattern. For example, in certain embodiments, the desired glycoprotein glycosylation pattern can be a modulation in afucosylation of the glycoprotein. In certain embodiments, a target range of afucosylated G0 (% G0-F or % normalized G0-F) can be between about 0% to about 20%, about 1% to about 15%, about 1% to about 10%, or about 1% to about 8%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%. In certain embodiments, the desired glycoprotein glycosylation pattern can be a modulation in galactosylation of the glycoprotein. For example, but not by way of limitation, a target range of agalactosylation (% G0) can be between about 40% to about 90%, about 50% to about 90%, about 50% to about 85%, or about 60% to about 80%. In certain embodiments, the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%. In certain embodiments, the desired glycoprotein glycosylation pattern can be a combination of modulation in afucosylation and galactosylation of the glycoprotein. For example, but not by way of limitation, a target range of % G0-F or % normalized G0-F can be between about 0% to about 20%, about 1% to about 15%, about 1% to about 10%, or about 1% to about 8%, and a target range of % G0 can be between about 40% to about 90%, about 50% to about 90%, about 50% to about 85%, or about 60% to about 80%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%, and the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%.

5.3 Sodium (Na+) Concentration

In certain embodiments, the present disclosure is directed to strategies for adjusting concentration of sodium (Na+) in cell culture media and/or cell cultures to modulate mAb glycosylation (e.g., galactosylation and/or fucosylation). For example, but not by way of limitation, cell culture media can be supplemented with Na₂CO₃, NaHCO₃, NaCl, NaOH, and/or Na+ compound (e.g., for pH control) or combination thereof, to achieve Na+ concentration target range of about 0 mM to about 250 mM, 20 mM to about 200 mM, 30 mM to about 150 mM, or 40 mM to about 130 mM.

Certain embodiments described herein relate to modulating glycosylation by adjusting the Na+ concentration in cell culture media and/or cell cultures to a specified target range to achieve or preserve a desired glycoprotein glycosylation pattern. In certain embodiments, the desired glycoprotein glycosylation pattern can be a modulation in afucosylation of the glycoprotein. For example, but not by way of limitation, a target range of afucosylation (% G0-F or % normalized G0-F) can be between about 0% to about 20%, about 1% to about 15%, about 1% to about 10%, or about 1% to about 8%. The % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%. In certain embodiments, the desired glycoprotein glycosylation pattern can be a modulation in galactosylation of the glycoprotein. For example, but not by way of limitation, a target range of agalactosylation (% G0) can be between about 40% to about 90%, about 50% to about 90%, about 50% to about 85%, or about 60% to about 80%. The % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%. In certain embodiments, the desired glycoprotein glycosylation pattern can be a combination of modulation in afucosylation and galactosylation of the glycoprotein. For example, but not by way of limitation, a target range of % G0-F or % normalized G0-F can be between about 0% to about 20%, about 1% to about 15%, about 1% to about 10%, or about 1% to about 8%, and a target range of G0 can be between about 40% to about 90%, about 50% to about 90%, about 50% to about 85%, or about 60%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%, and the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%.

5.4 Osmolality

In certain embodiments, the present disclosure is directed to strategies for adjusting osmolality of culture media to modulate mAb glycosylation (e.g., galactosylation and/or fucosylation). For example, but not by way of limitation, osmolality of cell culture media can be adjusted by adding sorbitol, KCl, an osmoprotectant (e.g., betaine), and/or NaCl to achieve osmolality target range of about 250 mOsm/kg to about 600 mOsm/kg, about 300 mOsm/kg to 450 mOsm/kg, about 325 mOsm/kg to 450 mOsm/kg, or about 325 mOsm/kg to 425 mOsm/kg.

Certain embodiments described herein relate to modulating glycosylation by adjusting osmolality level of culture media and/or cell cultures to the target ranges to achieve or preserve a desired glycoprotein glycosylation pattern. In certain embodiments, the desired glycoprotein glycosylation pattern can be a modulation in afucosylation of the glycoprotein. For example, but not by way of limitation, a target range of afucosylation (% G0-F or % normalized G0-F) can be between about 0% to about 20%, about 1% to about 15%, about 1% to about 10%, or about 1% to about 8%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%. In certain embodiments, the desired glycoprotein glycosylation pattern can be a modulation in galactosylation of the glycoprotein. For example, but not by way of limitation, a target range of agalactosylation (% G0) can be between about 40% to about 90%, about 50% to about 90%, about 50% to about 85%, or about 60% to about 80%. In certain embodiments, the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%. In certain embodiments, the desired glycoprotein glycosylation pattern can be a combination of modulation in afucosylation and galactosylation of the glycoprotein. For example, but not by way of limitation, a target range of % G0-F or % normalized G0-F can be between about 0% to about 20%, about 1% to about 15%, about 1% to about 10%, or about 1% to about 8%, and a target range of % G0 can be between about 40% to about 90%, about 50% to about 90%, about 50% to about 85%, or about 60% to about 80%. In certain embodiments, the % G0-F or % normalized G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%, and the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%.

5.5 Supplemental Manganese

As described in Sections 2 and 3, above, Mn concentration in cell culture media and/or cell cultures can impact glycosylation, e.g., mAb galactosylation and/or fucosylation. Accordingly, in certain embodiments, mAb glycosylation can be modulated not only by controlling for the amount of Mn present in cell culture media raw materials, as described above, but also by supplementing cell culture media and/or cell cultures with Mn to achieve specific Mn concentration targets or target ranges, including, as outlined in this Section, in combination with one or more other parameters. In certain embodiments, the concentration of Mn supplementation is selected to achieve a final target concentration or concentration range less than 10 uM (e.g., about 10 nM, 40 nM, 100 nM, 150 nM, 200 nM, 250 nM, 500 nM, 700 nM, 750 nM, 1000 nM, 1500 nM, 2000 nM, 3000 nM, 5000 nM, 8000 nM, or 10 uM, including concentrations falling within the ranges disclosed).

Certain embodiments described herein relate to modulating glycosylation by supplementing the disclosed concentrations of Mn into cell culture media and/or cell cultures to achieve or preserve a desired glycoprotein glycosylation pattern. In non-limiting embodiments, the desired glycoprotein glycosylation pattern can be a modulation in afucosylation of the glycoprotein. For example, but not by way of limitation, a target range of afucosylated G0 (% G0-F or % normalized G0-F) can be between about 0% to about 20%, about 1% to about 15%, about 1% to about 10%, or about 1% to about 8%. The % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%. In certain embodiments, the desired glycoprotein glycosylation pattern can be a modulation in galactosylation of the glycoprotein. For example, but not by way of limitation, a target range of agalactosylation (% G0) can be between about 40% to about 90%, about 50% to about 90%, about 50% to about 85%, or about 60% to about 80%. In certain embodiments, the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%.

In certain embodiments, the desired glycoprotein glycosylation pattern achieved by Mn supplementation can be a combination of a modulation in afucosylation and a modulation in fucosylation of the glycoprotein. For example, but not by way of limitation, a target range of % G0-F or % normalized G0-F can be between about 0% to about 20%, about 1% to about 15%, about 1% to about 10%, or about 1% to about 5%, and a target range of % G0 can be between about 40% to about 90%, about 50% to about 90%, about 50% to about 85%, or about 60% to about 80%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15% or about 20%, and the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%.

5.6 Combinations of pCO₂, Media Hold, Culture Duration, Supplemental Mn, Osmolality, and Na+ Concentration

In certain embodiments, combinations of the disclosed techniques to modulate glycosylation (e.g., afucosylation and/or galactosylation) of a glycoprotein can be used. For example, but not by way of limitation, combinations of the disclosed conditions of pCO₂, media hold, culture duration, supplemental Mn, osmolality, and/or Na+ concentration, can be employed in cell culture media and/or cell cultures to achieve or preserve a desired glycoprotein glycosylation pattern. In non-limiting embodiments, the disclosed media hold time (e.g., about 0 hours to about 72 hours) at a defined temperature or temperature range (e.g., about 25° C. to about 39° C.) can be applied to culture media in combination with supplemental Mn (e.g., about 1 nM to about 30000 nM), pCO₂ level (e.g., about 0 mmHg to about 250 mmHg), culture duration (e.g., about 0 day to about 25 days), Na+ concentration (e.g., about 0 mM to 250 mM), and osmolality (e.g., about 250 mOsm/kg to about 600 mOsm/kg).

In certain embodiments, combinations of the disclosed conditions of pCO₂, media hold, culture duration, supplemental Mn, osmolality, and Na+ concentration can induce combinatorial or synergistic effects with respect to the afucosylation and/or galactosylation profiles of a glycoprotein. For example, but not by way of limitation, synergistic modulations (e.g., increases or decreases) in the % G0-F of a glycoprotein can occur when combinations of the disclosed conditions of pCO₂, media hold, culture duration, supplemental Mn, osmolality, and Na+ concentration are employed. Moreover, in non-limiting embodiments, synergistic modulations (e.g., increases or decreases) in the % G0 of a glycoprotein can occur when combinations of the disclosed conditions of pCO₂, media hold, culture duration, supplemental Mn, osmolality, and Na+ concentration are employed. In addition, in certain embodiments, synergistic modulations in the % G0-F can occur when combinations of the disclosed conditions of pCO₂, media hold, culture duration, supplemental Mn, osmolality, and Na+ concentration are employed.

Certain embodiments described herein relate to modulating glycosylation by modifying combinations of the disclosed conditions (e.g., pCO₂, Media Hold, Culture Duration, Supplemental Mn, Osmolality, and Na+ concentration) to achieve or preserve a desired glycoprotein glycosylation pattern. In certain non-limiting embodiments, the desired glycoprotein glycosylation pattern can be an increase or an decrease in afucosylation of the glycoprotein. For example, but not by way of limitation, a target range of afucosylated G0 (% G0-F or % normalized G0-F) can be between about 0% to about 20%, about 1% to about 15%, about 1% to about 10%, or about 1% to about 8%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%. In certain embodiments, the desired glycoprotein glycosylation pattern can be a modulation in galactosylation of the glycoprotein. For example, but not by way of limitation, a target range of galactosylation (% G0) can be between about 40% to about 90%, about 50% to about 90%, about 50% to about 85%, or about 60% to about 80%. In certain embodiments, the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%. In certain embodiments, the desired glycoprotein glycosylation pattern can be a combination of modulation in afucosylation and galactosylation of the glycoprotein. In certain embodiments, a target range of % G0-F or % normalized G0-F can be between about 0% to about 20%, about 1% to about 15%, about 1% to about 10%, or about 1% to about 8%, and a target range of % G0 can be between about 40% to about 90%, about 50% to about 90%, about 50% to about 85%, or about 60% to about 80%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%, and the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%.

6. Galactose to Modulate Glycosylation

In certain embodiments, galactose, Mn, or a combination thereof in cell culture media and/or cell culture can impact glycosylation (e.g., galactosylation and afucosylation). Accordingly, mAb glycosylation can be modulated by supplementing galactose, Mn, or a combination thereof. For example, but not by way of limitation, the concentration of galactose can be added up to about 10 g/L (e.g., about 0 g/L, about 1.2 g/L, about 2 g/L, about 4 g/L, about 6 g/L, about 6.8 g/L, about 8 g/L, or about 10 g/L). In non-limiting embodiments, the concentration of galactose can be added up to about 100 mM. For example, but not by way of limitation, the concentration of galactose can be between about 0 mM to about 60 mM, about 0 mM to about 45 mM, about 0 mM to about 20 mM, or about 0 mM to about 10 mM. The cell culture can be further supplemented to achieve a Mn concentration of about 10 nM, about 40 nM, about 100 nM, about 150 nM, about 200 nM, about 250 nM, about 500 nM, about 700 nM, about 750 nM, about 1000 nM, about 1500 nM, about 2000 nM, about 3000 nM, about 5000 nM, about 8000 nM, or about 10 μM. The target concentration ranges of galactose and Mn can include concentrations falling within the ranges described. Non-limiting examples of galactose and Mn addition can include addition to the production culture and/or expansion cultures leading up to the production culture stage.

Certain embodiments described herein relate to modulating glycosylation by supplementing the disclosed concentrations of galactose with/without the disclosed concentrations of Mn into culture media to achieve or preserve a desired glycoprotein glycosylation pattern. In non-limiting embodiments, the desired glycoprotein glycosylation pattern can be a modulation in afucosylation of the glycoprotein. For example, but not by way of limitation, a target range of afucosylated G0 (% G0-F or % normalized G0-F) can be between about 0% to about 20%, about 1% to about 15%, about 1% to about 10%, or about 1% to about 8%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%. In certain embodiments, the desired glycoprotein glycosylation pattern can be a modulation in galactosylation of the glycoprotein. For example, but not by way of limitation, a target range of agalactosylation (% G0) can be between about 40% to about 90%, about 50% to about 90%, about 50% to about 85%, or about 60% to about 80%. In certain embodiments, the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%. In certain embodiments, the desired glycoprotein glycosylation pattern can be a combination of modulation in afucosylation and galactosylation of the glycoprotein. For example, but not by way of limitation, a target range of % G0-F or % normalized G0-F can be between about 0% to about 20%, about 1% to about 15%, about 1% to about 10%, or about 1% to about 8%, and a target range of % G0 can be between about 40% to about 90%, about 50% to about 90%, about 50% to about 85%, or about 60% to about 80%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%, and the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%.

7. Fucose and Cultivation Temperature, and their Combination to Modulate Glycosylation

In certain embodiments, addition of L-fucose (fucose) to the cell culture media and/or cell culture can also impact glycosylation (e.g., galactosylation and/or afucosylation). Accordingly, in certain embodiments, mAb glycosylation can be modulated by supplementing cell culture media with fucose. Addition of fucose results in a modulation in afucosylation (e.g., G0-F), and the extent to which afucosylation modulates can be refined by the fucose concentration and/or timing of fucose addition. In certain non-limiting embodiments, the concentration of fucose added can be between about 0 g/L and about 5 g/L (e.g., about 0 g/L, about 0.05 g/L, about 0.1 g/L, about 0.25 g/L, about 0.5 g/L, about 0.75 g/L, about 1 g/L, or about 5 g/L). In certain embodiments, the concentration of fucose can be added up to about 100 mM. For example, but not by way of limitation, the concentration of fucose can be between about 0 mM to about 100 mM, about 0 mM to about 30 mM, about 0 mM to about 10 mM, or about 0 mM to about 5 mM. In certain embodiments, fucose addition timing can be at between about 0 days and the end of the production culture (e.g., about 0 days, about 5 days, about 7 days, about 10 days, about 12 days, about 15 days, or about 25 days) after inoculation of the production culture with different fucose concentrations (e.g., about 0.1 g/L, about 0.5 g/L, including concentrations falling within the ranges disclosed). In certain embodiments, fucose addition at levels within a range of about 0 g/L to about 1 g/L can be performed in combination with culture temperatures within a range of about 25° C. to about 39° C. In certain embodiments, culture temperature can be between about 25° C. and about 39° C., about 30° C. to about 39° C., about 35° C. to about 39° C., or about 36° C. to about 39° C. In certain embodiments, fucose addition at levels of about 0 g/L to about 1 g/L or about 0 mM to about 60 mM can be performed in combination with Mn supplementation at levels of about 0 nM to 20000 nM in a low pCO₂ or high pCO₂ background. Non-limiting examples of fucose addition include addition to the production culture and/or expansion cultures leading up to the production culture stage.

Certain embodiments described herein relate to modulating glycosylation by supplementing the disclosed concentrations of fucose under disclosed conditions (e.g., pCO₂, supplemental Mn, etc.) into culture media to achieve or preserve a desired glycoprotein glycosylation pattern. In non-limiting embodiments, the desired glycoprotein glycosylation pattern that can achieved by increasing fucose concentration is a modulation (e.g., increase or decrease) in afucosylation of the glycoprotein. For example, but not by way of limitation, a target range of afucosylated G0 (% G0-F or % normalized G0-F) can be between about 0% to about 20%, about 1% to about 15%, about 1% to about 10%, or about 1% to about 8%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%. In certain embodiments, the desired glycoprotein glycosylation pattern achieved by increasing fucose concentration can be a modulation in galactosylation of the glycoprotein. For example, but not by way of limitation, a target range of fucosylated G0 (% G0) can be between about 40% to about 90%, about 50% to about 90%, about 50% to about 85%, or about 60% to about 80%. In certain embodiments, the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%. In certain embodiments, the desired glycoprotein glycosylation pattern achieved by increased fucose concentration can be a combination of a modulation in afucosylation and a modulation in galactosylation of the glycoprotein. For example, but not by way of limitation, a target range of % G0-F or % normalized G0-F can be between about 0% to about 20%, about 1% to about 15%, about 1% to about 10%, or about 1% to about 8%, and a target range of % G0 can be between about 40% to about 90%, about 50% to about 90%, about 50% to about 85%, or about 60% to about 80%. In certain embodiments, the % G0-F or % normalized G0-F can be modulated by about 1%, about 2%, about 3%, about 4%, about 5%, about 6%, about 7%, about 8%, about 9%, about 10%, about 11%, about 12%, about 13%, about 14%, about 15%, or about 20%, and the % G0 can be modulated by about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, or about 50%.

8. Cell Culture and Glycoprotein Compositions

In certain embodiments the present disclosure relates to compositions of glycoproteins, e.g., mAbs, obtained via the use of the cell culture strategies outlined herein. Such compositions can comprise specific cell culture compositions defined by the nature of the cell culture media, host cells, and glycoprotein being expressed. For example, but not by way of limitation, the compositions of the present disclosure are directed to compositions of a glycoprotein of interest, e.g., a mAb, exhibiting a particular glycosylation profile, e.g., a particular amount of fucosylated and/or galactosylated G0 glycans. In certain embodiments, the compositions of the present disclosure are directed to compositions of cell culture media either containing or having been supplemented to contain advantageous Mn concentrations. In certain embodiments, the present disclosure can be directed to a combination of such cell culture media and such glycoproteins of interest exhibiting a particular glycosylation profile.

In certain embodiments the present disclosure relates to compositions of glycoproteins, e.g., mAbs, obtained by screening and selecting cell culture media for compliance with specific action targets, or otherwise controlling for cell culture media component variation. For example, but not by way of limitation, the present disclosure is directed to mAb compositions wherein the composition results from a cell culture in which the cell culture media Mn concentration falls within the range of 30 nM to 110 nM, and where media falling outside of such a range in Mn concentration is not employed in connection with cell culture producing the mAb. The present disclosure is also directed to mAb compositions wherein the composition results from a cell culture in which the cell culture Mn concentration falls within the range of 30 nM to 110 nM, and where cell cultures falling outside of such a range in Mn concentration are not employed in connection with cell culture producing the mAb. In certain embodiments, the compositions of the present disclosure are directed to compositions of cell culture media where the Mn concentration(s) of raw material(s) has been screened and/or selected. In certain embodiments, the present disclosure can be directed to a combination of such cell culture media and such glycoproteins of interest exhibiting a particular glycosylation profile.

In certain embodiments the present disclosure relates to compositions of glycoproteins, e.g., mAbs, exhibiting particular glycosylation profiles, e.g., a particular amount of fucosylated and/or galactosylated G0 glycans, obtained by controlling the concentration of Mn in the cell culture media via performing HTST treatment of the media with a pre-HTST pH adjustment target of less than about 7.3 or less than about 7.0. For example, but not by way limitation, the pre-HTST pH adjustment target for the media can be about 6.1, about 6.3, about 6.5, about 6.6, about 6.7, about 6.8, about 6.9, about 7.0, about 7.1, about 7.2, or about 7.3. In certain embodiments, the compositions of the present disclosure are directed to compositions of cell culture media where the HTST treatment step has been performed with a pre-HTST pH adjustment target as disclosed herein. In certain embodiments, the present disclosure can be directed to a combination of such cell culture media and such glycoproteins of interest exhibiting a particular glycosylation profile.

In certain embodiments the present disclosure relates to compositions of glycoproteins, e.g., mAbs, exhibiting particular glycosylation profiles, e.g., a particular amount of fucosylated and/or galactosylated G0 glycans, obtained by controlling the pCO₂, media hold duration, culture duration, cultivation temperature, manganese, galactose, fucose and/or osmolality/Na+ concentration, or a combination thereof in cell culture processes, as outlined herein. In certain embodiments, the compositions of the present disclosure are directed to compositions of cell culture media where the pCO₂, media hold duration, culture duration, cultivation temperature, manganese, galactose, fucose and/or osmolality/Na+ concentration, or a combination thereof, have been controlled as outlined herein. In certain embodiments, the present disclosure can be directed to a combination of such cell culture media and such glycoproteins of interest exhibiting a particular glycosylation profile.

In certain embodiments, various bioreactor configurations can be used for the compositions of cell culture media. For example, but not by way of limitation, volume of the bioreactor can be between about 1 L and about 20,000 L (e.g., about 1 L, about 1.5 L, about 2 L, about 5 L, about 10 L, about 50 L, about 100 L, about 250 L, about 500 L, about 1000 L, about 2000 L, about 3000 L, about 4000 L, about 5000 L, about 6000 L, about 7000 L, about 8000 L, about 9000 L, about 10,000 L, about 11,000 L, about 12,000 L, about 13,000 L, about 14,000 L, about 15,000 L, about 16,000 L, about 17,000 L, about 18,000 L, about 19,000 L, or about 20,000 L). In non-limiting embodiments, bioreactor configurations can be modified to adjust levels of pCO₂, medial hold duration, osmolality, Na+, Mn, temperature, pH, fucose, galactose, or combinations thereof.

In addition to the various embodiments depicted and claimed, the disclosed subject matter is also directed to other embodiments having other combinations of the features disclosed and claimed herein. As such, the features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the compositions and methods of the disclosed subject matter without departing from the spirit or scope of the disclosed subject matter. Thus, it is intended that the disclosed subject matter include modifications and variations that are within the scope of the appended claims and their equivalents. Various publications, patents and patent applications are cited herein, the contents of which are hereby incorporated by reference in their entireties.

### EMBODIMENTS OF THE DISCLOSURE

The following are non-limiting embodiments of the instant disclosure.
1. A method for modulating the glycosylation pattern of a glycoprotein of interest in a cell culture, comprising: modulating the following parameters, either alone or in any combination, in a cell culture medium, and/or, in a cell culture environment: a Mn concentration from about 1 nM to about 20000 nM in a high partial pressure CO₂ (pCO₂) condition; a Mn concentration from about 1 nM to about 30000 nM in a low pCO₂ condition; a pCO₂ from about 10 mmHg to about 250 mmHg; a pre-inoculation cell culture media hold duration from about 0 hrs to about 120 hrs at a temperature of about 25° C. to 39° C.; a cell culture duration from about 0 days to about 150 days; a Na+ concentration from about 0 mM to about 300 mM; an osmolality from about 250 mOsm/kg to about 550 mOsm/kg; a galactose concentration from about 0 mM to about 60 mM; a fucose concentration from about 0 mM to about 60 mM; and a cultivation temperature from about 29° C. to about 39° C.
2. The method of embodiment 1, wherein the cell culture environment is in a bioreactor with or without cells.
3. The method of embodiment 1 or embodiment 2, wherein the low pCO₂ condition is from about 10 to about 100 mmHg, and the high pCO₂ condition is from about 20 to about 250 mmHg.
4. The method of embodiment 3, wherein the duration of pCO₂ modulation covers at least the first half of the cell culture duration.
5. The method of any one of the preceding embodiments, wherein the glycoprotein of interest is a recombinant protein.
6. The method of any one of the preceding embodiments, wherein the recombinant protein is an antibody or antibody fragment, a scFv (single-chain variable fragment), BsDb (bispecific diabody), scBsDb (single-chain bispecific diabody), scBsTaFv (single-chain bispecific tandem variable domain), DNL-(Fab)3 (dock-and-lock trivalent Fab), sdAb (single-domain antibody) and BssdAb (bispecific single-domain antibody).
7. The method of any one of the preceding embodiments, wherein the antibody is a chimeric, a humanized or a human antibody.
8. The method of any one of the preceding embodiments, wherein the antibody is an anti-CD20 antibody.
9. The method of any one of the preceding embodiments, wherein the anti-CD20 antibody is ocrelizumab.
10. The method of embodiments 6 to 8, wherein the antibody or antibody fragment exhibits: a % G0-F (percent afucosylated glycoprotein) between about 0% to about 20%; about 1% to about 15%; about 1% to about 10%; or about 1% to about 8%; or, a normalized % G0-F between about 0% to about 20%; about 1% to about 15%; about 1% to about 10%; or about 1% to about 8%; and/or, a % G0 (percent agalactosylated glycoprotein) between about 40% to about 90%; about 50% to about 90%; about 55% to about 85%; or about 60% to about 80%.
11. The method of embodiments 6 to 9, wherein the glycosylation is modulated to achieve: an increased afucosylation (e.g., G0-F (afucosylated G0)), while decreasing agalactosylation (e.g., G0 (fucosylated, agalactosylated G0)); or, a decreased afucosylation (e.g., G0-F), while increasing agalactosylation (e.g., G0); or, an increased or decreased afucosylation (e.g., G0-F) without impacting agalactosylation (e.g., G0); or, an increased or decreased agalactosylation (e.g., G0) without impacting afucosylation (e.g., G0-F).
12. The method of any one of the preceding embodiments, wherein the step of modulating the glycosylation pattern of the glycoprotein of interest comprises: modulating the Mn concentration from about 1 nM to about 30000 nM under a low pCO₂ condition, or, modulating the Mn concentration from about 1 nM to about 20000 nM under a high pCO₂ condition, and the following parameters, either alone or in any combination, in the cell culture medium, and/or in the cell culture environment: the pre-inoculation cell culture media hold duration from about 0 hrs to about 120 hrs at a temperature of about 25° C. to 39° C.; the cell culture duration from about 0 days to about 150 days; the Na+ concentration from about 0 mM to about 300 mM; the osmolality from about 250 mOsm/kg to about 550 mOsm/kg; the galactose concentration from about 0 mM to about 60 mM; the fucose concentration from about 0 mM to about 60 mM; and the cultivation temperature from about 29° C. to about 39° C.
13. The method of embodiment 12, wherein the step of modulating the glycosylation pattern of the glycoprotein of interest comprises: modulating the Mn concentration from about 1 nM to about 30000 nM under a low pCO₂ condition, or, modulating the Mn concentration from about 1 nM to about 20000 nM under a high pCO₂ condition, and the following parameters in the cell culture medium, and/or in the cell culture environment: the pre-inoculation cell culture media hold duration from about 0 hrs to about 120 hrs at a temperature of about 25° C. to 39° C.; and the cell culture duration from about 0 days to about 150 days.
14. The method of embodiment 12, wherein the step of modulating the glycosylation pattern of the glycoprotein of interest comprises: modulating the Mn concentration from about 1 nM to about 30000 nM under a low pCO₂ condition, or, modulating the Mn concentration from about 1 nM to about 20000 nM under a high pCO₂ condition, and the following parameters in the cell culture medium, and/or in the cell culture environment: the galactose concentration from about 0 mM to about 60 mM; and/or, the fucose concentration from about 0 mM to about 60 mM.
15. The method of any one of the preceding embodiments, wherein the step of modulating the glycosylation pattern of the glycoprotein of interest comprises: modulating the pre-inoculation cell culture media hold duration and any of the following parameters, either alone or in any combination, in the cell culture medium, and/or in the cell culture environment: a Mn concentration from about 1 nM to about 20000 nM in a high partial pressure CO₂ (pCO₂) condition; a Mn concentration from about 1 nM to about 30000 nM in a low pCO₂ condition; a pCO₂ from about 10 mmHg to about 250 mmHg; a cell culture duration from about 0 days to about 150 days; a Na+ concentration from about 0 mM to about 300 mM; an osmolality from about 250 mOsm/kg to about 550 mOsm/kg; a galactose concentration from about 0 mM to about 60 mM; a fucose concentration from about 0 mM to about 60 mM; and a cultivation temperature from about 29° C. to about 39° C.; wherein the cell culture media hold duration is from about 0 hrs to about 120 hrs at a temperature of about 25° C. to 39° C.
16. The method of embodiment 15, wherein the step of modulating the glycosylation pattern of the glycoprotein of interest comprises: modulating the pre-inoculation cell culture media hold duration and the following parameters in the cell culture medium, and/or in the cell culture environment: the Mn concentration from about 1 nM to about 20000 nM in a high partial pressure CO₂ (pCO₂) condition, or, a Mn concentration from about 1 nM to about 30000 nM in a low pCO₂ condition; the pCO₂ from about 10 mmHg to about 250 mmHg; and, the cell culture duration from about 0 days to about 150 days;
   wherein the cell culture media hold duration is from about 0 hrs to about 120 hrs at a temperature of about 25° C. to 39° C.
17. The method of any one of embodiments 12-15, wherein the glycoprotein of interest is an antibody or an antibody fragment thereof
18. The method of embodiment 17, wherein the antibody or the antibody fragment thereof is an anti-CD20 antibody.
19. The method of embodiment 18, wherein the anti-CD20 antibody is ocrelizumab.
20. The method of any one of the preceding embodiments, wherein the step of modulating the glycosylation pattern of the glycoprotein of interest comprises: modulating the cell culture duration and any of the following parameters, either alone or in any combination, in the cell culture medium, and/or in the cell culture environment: a Na+ concentration from about 0 mM to about 300 mM; an osmolality from about 250 mOsm/kg to about 550 mOsm/kg; a galactose concentration from about 0 mM to about 60 mM; a fucose concentration from about 0 mM to about 60 mM; and a cultivation temperature from about 29° C. to about 39° C., wherein the cell culture duration is from about 0 days to about 150 days.
21. The method of any one of the preceding embodiments, wherein the step of modulating the glycosylation pattern of the glycoprotein of interest comprises: modulating the Na+ concentration of about 0 nM to about 300 nM and any of the following parameters, either alone or in any combination, in the cell culture medium, and/or in the cell culture environment: an osmolality from about 250 mOsm/kg to about 550 mOsm/kg; a galactose concentration from about 0 mM to about 60 mM; a fucose concentration from about 0 mM to about 60 mM; and a cultivation temperature from about 29° C. to about 39° C., wherein the Na+ concentration from about 0 mM to about 300 mM.
22. The method of any one of the preceding embodiments, wherein the step of modulating the glycosylation pattern of the glycoprotein of interest comprises: modulating the Na+ concentration and the pCO₂ from about 10 mmHg to about 250 mmHg.
23. The method of any one of the preceding embodiments, wherein the step of modulating the glycosylation pattern of the glycoprotein of interest comprises: modulating the osmolality and any of the following parameters, either alone or in any combination, in the cell culture medium, and/or in the cell culture environment: a galactose concentration from about 0 mM to about 60 mM; a fucose concentration from about 0 mM to about 60 mM; and a cultivation temperature from about 29° C. to about 39° C., wherein the osmolality is from about 250 mOsm/kg to about 550 mOsm/kg.
24. The method of embodiment 1, wherein the step of modulating the glycosylation pattern of the glycoprotein of interest comprises: modulating the Mn concentration from about 1 nM to about 30000 nM under a low pCO₂ condition, or modulating the Mn concentration from about 1 nM to about 20000 nM under a high pCO₂ condition, modulating the Na+ concentration from about 0 mM to about 300 mM, and modulating the duration of the pre-inoculation cell culture media hold from about 0 hrs to about 120 hrs.
25. The method of embodiment 1, wherein modulation of the glycosylation pattern of the glycoprotein of interest comprises modulating the osmolality from about 250 mOsm/kg to about 550 mOsm/kg and the pCO₂ from about 10 mmHg to about 250 mmHg.
26. The method of embodiment 1, wherein the modulation of the glycosylation pattern of the glycoprotein of interest comprises modulating: the cultivation temperature from about 29° C. to about 39° C., and, the galactose concentration from about 0 mM to about 60 mM; and/or the fucose concentration from about 0 mM to about 60 mM.
27. The method of embodiment 1, wherein modulation of the glycosylation pattern of the glycoprotein of interest comprises modulating the pCO₂ from about 10 mmHg to about 250 mmHg and the fucose concentration from about 0 mM to about 60 mM.
28. The method of embodiment 1, wherein modulation of the glycosylation pattern of the glycoprotein of interest comprises modulating the fucose concentration from about 0 mM to about 60 mM and the cultivation temperature from about 29° C. to about 39° C.
29. The method of embodiment 1, wherein the modulation of the glycosylation pattern of the glycoprotein of interest comprises: modulating a pCO₂ concentration and any of the following parameters, either alone or in any combination, in the cell culture medium, and/or in the cell culture environment: a Mn concentration from about 1 nM to about 20000 nM in a high partial pressure CO₂ (pCO₂) condition; a Mn concentration from about 1 nM to about 30000 nM in a low pCO₂ condition; a pre-inoculation cell culture media hold duration from about 0 hrs to about 120 hrs at a temperature of about 25° C. to 39° C.; a cell culture duration from about 0 days to about 150 days; a Na+ concentration from about 0 mM to about 300 mM; an osmolality from about 250 mOsm/kg to about 550 mOsm/kg; a galactose concentration from about 0 mM to about 60 mM; a fucose concentration from about 0 mM to about 60 mM; and a cultivation temperature from about 29° C. to about 39° C., wherein the pCO₂ concentration is from about 10 mmHg to about 250 mmHg.
30. The method of any one of the above embodiments, wherein the Mn concentration is from about 1 nM to about 20000 nM in a high pCO₂ culture; from about 1 nM to about 10000 nM, from about 1 nM to about 5000 nM, from about 1 nM to about 4000 nM, from about 1 nM to about 3000 nM, from about 1 nM to about 2000 nM, from about 1 nM to about 1000 nM in a high pCO₂ culture; from about 1 nM to about 500 nM, from about 1 nM to about 100 nM, from about 1 nM to about 50 nM, from about 1 nM to about 20 nM, from about 20 nM to about 2000 nM, from about 20 nM to about 3000 nM, from about 20 nM to about 10000 nM, from about 20 nM to about 20,000 nM, from about 20 nM to about 300 nM, about 30 nM to about 110 nM in a high pCO₂ culture.
31. The method of any one of the above embodiments, wherein the Mn concentration is about 1 nM to about 30000 nM in a low pCO₂ culture; from about 1 nM to about 20000 nM; from about 1 nM to about 10000 nM, from about 1 nM to about 5000 nM, from about 1 nM to about 4000 nM, from about 1 nM to about 3000 nM, from about 1 nM to about 2000 nM, from about 1 nM to about 1000 nM; from about 1 nM to about 500 nM, from about 1 nM to about 100 nM, from about 1 nM to about 50 nM, from about 1 nM to about 20 nM, from about 20 nM to about 100 nM, about 20 nM to about 300 nM, from about 20 nM to about 500 nM, from about 20 nM to about 1000 nM, from about 20 nM to about 2000 nM, from about 20 nM to about 3000 nM, from about 20 nM to about 5000 nM, from about 20 nM to about 10000 nM, from about 20 nM to about 20000 nM, or about 30 nM to about 110 nM in a low pCO₂ culture.
32. The method of any one of the above embodiments, wherein modulation of the Mn concentration comprises determining the Mn content in cell culture raw materials and selecting raw material lots to modulate the Mn concentration.
33. The method of any one of the above embodiments, wherein modulation of the Mn concentration comprises (i) controlling materials that come into contract with cell culture media or cell culture; or (ii) accounting for the concentration of leached Mn in cell culture media or during cell culture; or a combination of (i) and (ii) to modulate the Mn concentration.
34. The method of embodiment 33, wherein the leached Mn is produced by contact of the cell culture and/or cell culture media with: (i) a filter; (ii) a media preparation, hold, or culture vessel; or (iii) combinations of (i) and (ii).
35. The method of embodiment 34, wherein the filter includes but is not limited to: a depth filter, a column, a membrane and a disc.
36. The method of embodiment 34, wherein the filter material includes but is not limited to: diatomaceous earth, hollow fibers or a resin.
37. The method of any one of the preceding embodiments, wherein the cell culture medium is a basal medium, a reconstituted medium, a feed medium, a hydrolysate, a supplement, serum or an additive.
38. The method of any one of the preceding embodiments, wherein the cell culture medium is supplemented during the production stage of the cell culture.
39. The method of any one of the preceding embodiments, wherein the cell culture medium is supplemented prior to the production stage of the cell culture.
40. The method of any one of the preceding embodiments, wherein the cell culture medium comprises one or more of: Mn, fucose, galactose and/or Na+, and wherein the supplementation is based on a pre-defined schedule or criteria.
41. The method of any one of the preceding embodiments, wherein the one or more of the Mn, fucose, galactose and Na+ is supplemented as a bolus, as an intermittent supplement, as a continuous supplement, as a semi-continuous supplement, as a feedback loop-based supplement, or as a combination of one or more of thereof.
42. The method of any one of the preceding embodiments, wherein the cell culture medium consists essentially of one or more of: i) Mn; ii) fucose; iii) galactose; and/or iv) Na+.
43. The method of any one of the preceding embodiments, wherein the modulation of the Mn concentration comprises employing a cell culture media pH of about 6.1 to about 7.3; or about 6.3 to about 7.3 prior to High Temperature Short Time (HTST) heat treatment.
44. The method of any one of the preceding embodiments, wherein modulation of the glycosylation pattern of the glycoprotein of interest comprises modulating the pCO₂.
45. The method of any one of the preceding embodiments, wherein the cell culture or cell culture media is in a bioreactor and where modulation of pCO₂ is achieved by modulating: the bioreactor working volume; the bioreactor gas sparging strategy; the bioreactor agitation strategy; the bioreactor media exchange strategy, the bioreactor perfusion strategy, the bioreactor feed strategy, or an any combination thereof.
46. The method of embodiment 44, wherein the pCO₂ is modulation comprises establishing a high pCO₂ culture.
47. The method of embodiment 46, wherein the pCO₂ is about 20 mmHg to about 250 mmHg; about 20 mmHg to about 250 mmHg; about 20 mmHg to about 150 mmHg; or about 30 mmHg to about 250 mmHg.
48. The method of any one of the preceding embodiments, wherein the pCO₂ is modulation comprises establishing a low pCO₂ culture.
49. The method of embodiment 48, wherein the pCO₂ is about 10 mmHg to about 100 mmHg; 10 mmHg to about 80 mmHg; about 20 mmHg to about 70 mmHg; or about 30 mmHg to about 60 mmHg.
50. The method of any one of the preceding embodiments, wherein the pCO₂ modulation occurs at day 0 of the culture.
51. The method of embodiment 50, wherein the pCO₂ modulation occurs for: about the majority of the cell culture; about the first 5 days; about the first 7 days; or about the first 10 days.
52. The method of embodiment 50, wherein the pCO₂ modulation occurs for: about the majority of the production culture; about the first 5 days; about the first 7 days; or about the first 10 days.
53. The method of embodiment 52, wherein modulation of the glycosylation pattern of the glycoprotein of interest comprises modulating the duration of the pre-inoculation cell culture media hold, wherein the duration of the pre-inoculation cell culture media hold is about 0 hrs to about 120 hrs; about 0 hrs to about 72 hrs; about 0 hrs to about 48 hrs; or about 0 hrs to about 24 hrs.
54. The method of embodiment 53, wherein the temperature of the media during the pre-inoculation cell culture media hold is about 25° C. to about 39° C.; about 30° C. to about 39° C.; about 35° C. to about 39° C.; or about 36° C. to about 39° C.
55. The method of any one of the preceding embodiments, wherein modulation of the glycosylation pattern of the glycoprotein of interest comprises modulating the duration of the cell culture, wherein the duration of the cell culture is about 0 days to about 150 days; about 0 days to about 15 days; about 0 days to about 12 days; 0 days to about 7 days; or about 0 days to about 5 days.
56. The method of any one of the preceding embodiments, wherein modulation of the glycosylation pattern of the glycoprotein of interest comprises modulating the Na+ concentration, wherein the Na+ concentration is about 0 mM to about 300 mM; is about 20 mM to about 20 mM; about 30 mM to about 150 mM; or about 40 mM to about 130 mM.
57. The method of any one of the preceding embodiments, wherein the modulation of the Na+ concentration comprises supplementing the cell culture with Na compounds including but not limited to: Na₂CO₃, NaHCO₃, NaOH, NaCl, or combinations thereof
58. The method of any one of the above embodiments, wherein modulation of the glycosylation pattern of the glycoprotein of interest comprises modulating the osmolality, wherein the osmolality of is about 250 mOsm/kg to about 550 mOsm/kg; about 300 mOsm/kg to about 450 mOsm/kg; or about 325 mOsm/kg to about 425 mOsm/kg.
59. The method of any one of the preceding embodiments, wherein the modulation of the osmolality comprises supplementing the cell culture with an osmolality-modulating media component.
60. The method of embodiment 59, wherein the osmolality-modulating media component is NaCl, KCl, sorbitol, an osmoprotectant, or combinations thereof
61. The method of embodiment 59, wherein the osmolality-modulating media component is supplemented as a bolus, as an intermittent supplement, as a continuous supplement, as a semi-continuous supplement, as a feedback loop-based supplement, or as a combination of one or more of thereof.
62. The method of any one of the preceding embodiments, wherein modulation of the glycosylation pattern of the glycoprotein of interest comprises modulating the galactose concentration, wherein the galactose concentration is about 0 mM to about 60 mM or about 0 mM to about 50 mM.
63. The method of any one of the preceding embodiments, wherein modulation of the glycosylation pattern of the glycoprotein of interest comprises modulating the fucose concentration, wherein the fucose concentration is about 0 mM to about 60 mM; 0 mM to about 40 mM; about 0 mM to about 20 mM; or about 0 mM to about 10 mM.
64. The method of any one of the preceding embodiments, wherein modulation of the glycosylation pattern of the glycoprotein of interest comprises modulating the cell culture temperature, wherein the cell culture temperature is about 29° C. to about 39° C.; about 30° C. to about 39° C.; about 31° C. to about 38° C.; or about 34° C. to about 38° C.
65. The method of embodiment 64, wherein the cell culture temperature is modulated during the production stage of the cell culture.
66. The method of any one of the preceding embodiments, wherein the cell culture temperature is modulated prior the production stage of the cell culture.
67. The method of any one of the preceding embodiments, wherein the cell culture temperature is modulated based on a pre-defined schedule or criteria.
68. The method of any one of the preceding embodiments, wherein the cell culture comprises eukaryotic cells.
69. The method of embodiment 68, wherein the eukaryotic cells are insect, avian, fungal, plant or mammalian cells.
70. The method of embodiment 69, wherein the fungal cells are yeast, Pichia or any filamentous fungal cells
71. The method of embodiment 70, wherein the yeast cells are S. cerevisiae cells.
72. The method of embodiment 69, wherein the mammalian cells are CHO cells.
73. The method of any one of the preceding embodiments, wherein the cell culture is in a bioreactor including but not limited to: a single use technology (SUT) bag or bioreactor; a WAVE bioreactor; a stainless steel bioreactor; a flask; a tube and a chamber.
74. The method of any one of the preceding embodiments, wherein the volume of the cell culture is from 1 mL to 35,000 L.
75. The method of embodiment 74, wherein the volume of the cell culture is from 1 mL to 10 ml, from 1 mL to 50 ml, from 1 mL to 100 ml, from 1 mL to 200 ml, from 1 mL to 300 ml, from 1 mL to 500 ml, from 1 mL to 1000 ml, from 1 mL to 2000 ml, from 1 mL to 3000 ml, from 1 mL to 4000 ml, from 1 mL to 5000 ml, from 1 mL to 1 L, from 1 mL to 2 L, from 1 mL to 3 L, from 1 mL to 4 L, from 1 mL to 5 L, from 1 mL to 6 L, from 1 mL to 10 L, from 1 mL to 20 L, from 1 mL to 30 L, from 1 mL to 40 L, from 1 mL to 50 L, from 1 mL to 60 L, from 1 mL to 70 L, from 1 mL to 100 L, from 1 mL to 200 L, from 1 mL to 300 L, from 1 mL to 400 L, from 1 mL to 500 L, from 1 mL to 1000 L, from 1 mL to 2000 L, from 1 mL to 3000 L, from 1 mL to 4000 L, from 1 mL to 5000 L, from 1 mL to 10,000 L, from 1 mL to 20,000 L, from 1 mL to 30,000 L, from 1 mL to 30,000 L, from 1 mL to 35,000 L.
76. Use of a method of any one of embodiments 1-75 to obtain a glycoprotein of interest exhibiting: a % G0-F (percent afucosylated glycoprotein) between about 0% to about 20%; about 1% to about 15%; about 1% to about 10%; or about 1% to about 8%; or, a normalized % G0-F between about 0% to about 20%; about 1% to about 15%; about 1% to about 10%; or about 1% to about 8%; and/or, a % G0 (percent agalactosylated glycoprotein) between about 40% to about 90%; about 50% to about 90%; about 55% to about 85%; or about 60% to about 80%.
77. The use of a method of any one of embodiments 1-75 wherein the glycosylation is modulated to achieve: an increased afucosylation (e.g., G0-F (afucosylated G0)), while decreasing agalactosylation (e.g., G0 (fucosylated, agalactosylated G0)); or, a decreased afucosylation (e.g., G0-F), while increasing agalactosylation (e.g., G0); or, an increased or decreased afucosylation (e.g., G0-F) without impacting agalactosylation (e.g., G0); or, an increased or decreased agalactosylation (e.g., G0) without impacting afucosylation (e.g., G0-F).
78. A method to prepare a cell culture media, a feed media, a hydrolysate, or an additive comprising one or more step(s) of modulating: the Mn concentration in a high partial pressure CO₂ (pCO₂) culture from about 1 nM to about 20000 nM; the Mn concentration in a low pCO₂ culture from about 1 nM to about 30000 nM; the pCO₂ from about 10 mmHg to about 250 mmHg; the pre-inoculation cell culture media hold duration from about 0 hrs to about 120 hrs; the cell culture duration from about 0 days to about 150 days; the Na+ concentration from about 0 mM to about 300 mM; the osmolality from about 250 mOsm/kg to about 550 mOsm/kg; the galactose concentration from about 0 mM to about 60 mM; the fucose concentration from about 0 mM to about 60 mM; and the cultivation temperature from about 29° C. to about 39° C.; wherein the cell culture media, feed media, hydrolysate, or additive modulates the glycosylation pattern of a glycoprotein of interest.
79. The method of embodiment 78, wherein the glycoprotein of interest is an antibody or antibody fragment.
80. The method of embodiment 79, wherein the antibody or antibody fragment exhibits: a % G0-F between about 0% to about 20%; about 1% to about 15%; about 1% to about 10%; or about 1% to about 8%; or a normalized % G0-F between about 0% to about 20%; about 1% to about 15%; about 1% to about 10%; or about 1% to about 8%; and/or a % G0 between about 40% to about 90%; about 50% to about 90%; about 55% to about 85%; or about 60% to about 80%.
81. The method of embodiment 79, wherein the glycosylation of the antibody or antibody fragment is modulated to achieve: an increased afucosylation (e.g., G0-F (afucosylated G0)), while decreasing agalactosylation (e.g., G0 (fucosylated, agalactosylated G0)); or, a decreased afucosylation (e.g., G0-F), while increasing agalactosylation (e.g., G0); or, an increased or decreased afucosylation (e.g., G0-F) without impacting agalactosylation (e.g., G0); or, an increased or decreased agalactosylation (e.g., G0) without impacting afucosylation (e.g., G0-F).
82. The method of any one of embodiments 78-81, comprising modulating the Mn concentration from about 1 nM to about 30000 nM and the duration of the pre-inoculation cell culture media hold from about 0 hrs to about 120 hrs.
83. The method of any one of embodiments 78-82, comprising modulating the pCO₂ from about 10 mmHg to about 250 mmHg, the Na+ concentration from about 0 mM to about 300 mM, and the duration of the pre-inoculation cell culture media hold from about 0 hrs to about 120 hrs.
84. The method of any one of embodiments 78-83, comprising modulating the Mn concentration from about 1 nM to about 30000 nM, the pCO₂ from about 10 mmHg to about 250 mmHg, and the Na+ concentration from about 0 mM to about 300 mM.
85. The method of any one of embodiments 78-84, comprising modulating the Mn concentration from about 1 nM to about 30000 nM, the pCO₂ from about 10 mmHg to about 250 mmHg, the Na+ concentration from about 0 mM to about 300 mM, and the duration of the pre-inoculation cell culture media hold from about 0 hrs to about 72 hrs.
86. The method of any one of embodiments 78-85, comprising modulating the pCO₂ from about 10 mmHg to about 250 mmHg and the Na+ concentration from about 0 mM to about 300 mM.
87. The method of any one of embodiments 78-86, comprising modulating the osmolality from about 250 mOsm/kg to about 550 mOsm/kg and the pCO₂ from about 10 mmHg to about 250 mmHg.
88. The method of any one of embodiments 78-87, comprising modulating the pCO₂ from about 10 mmHg to about 250 mmHg, the Mn concentration from about 1 nM to about 30000 nM, the duration of the cell culture from about 0 days to about 150 days, and the duration of the pre-inoculation cell culture media hold from about 0 hrs to about 120 hrs
89. The method of any one of embodiments 78-88, comprising modulating the Mn concentration from about 1 nM to about 30000 nM and the galactose concentration from about 0 mM to about 60 mM.
90. The method of any one of embodiments 78-89, comprising modulating the fucose concentration from about 0 mM to about 60 mM and the Mn concentration from about 1 nM to about 30000 nM.
91. The method of any one of embodiments 78-90, comprising modulating the fucose concentration from about 0 mM to about 60 mM and the pCO₂ from about 10 mmHg to about 250 mmHg.
92. The method of any one of embodiments 78-91, comprising modulating the fucose concentration from about 0 mM to about 60 mM, the Mn concentration from about 1 nM to about 30000 nM, and the pCO₂ from about 10 mmHg to about 250 mmHg.
93. The method of any one of embodiments 78-92, comprising modulating the fucose concentration from about 0 mM to about 60 mM and the cell culture temperature is about 29° C. to about 39° C.
94. The method of any one of embodiments 78-93, comprising modulating the fucose concentration from about 0 mM to about 60 mM and the duration of the cell culture from about 0 days to about 150 days.
95. The method of any one of embodiments 78-94, wherein the Mn concentration is about 1 nM to about 20000 nM in a high pCO₂ culture; about 1 nM to about 1000 nM in a high pCO₂ culture; about 20 nM to about 300 nM in a high pCO₂ culture; or about 30 nM to about 110 nM in a high pCO₂ culture.
96. The method of any one of embodiments 78-95, wherein the Mn concentration is about 1 nM to about 30000 nM in a low pCO₂ culture; about 1 nM to about 3000 nM in a low pCO₂ culture; about 20 nM to about 300 nM in a low pCO₂ culture; or about 30 nM to about 110 nM in a low pCO₂ culture.
97. The method of embodiment 95 or embodiment 96, wherein modulation of the Mn concentration comprises determining the Mn content in cell culture raw materials and selecting raw material lots to modulate the Mn concentration.
98. The method of embodiment 95 or embodiment 96, wherein modulation of the Mn concentration comprises i) controlling materials that come into contract with cell culture media or cell culture; or (ii) accounting for the concentration of leached Mn in cell culture media or during cell culture; or a combination of (i) and (ii) to modulate the Mn concentration.
99. The method of embodiment 98, wherein the leached Mn is produced by contact of the cell culture and/or cell culture media with: (i) a filter; (ii) a media preparation, hold, or culture vessel; or (iii) combinations of (i) and (ii).
100. The method of embodiment 99, wherein the filter includes but is not limited to: a depth filter, a column, a membrane and a disc.
101. The method of embodiment 99, wherein the filter material includes but is not limited to: diatomaceous earth, hollow fibers or a resin.
102. The method of embodiment 95 or embodiment 96, wherein the modulation of the Mn concentration comprises employing a cell culture media pH of about 6.1 to about 7.3; or about 6.3 to about 7.3 prior to HTST treatment.
103. The method of any one of embodiments 78-102, wherein the pCO₂ is modulated.
104. The method of embodiment 103, wherein the cell culture media is in a bioreactor and where modulation of pCO₂ is achieved by modulating: the bioreactor working volume; the bioreactor gas sparging strategy; the bioreactor agitation strategy; the bioreactor feed strategy; the bioreactor perfusion strategy; the bioreactor media exchange strategy; or any combination thereof
105. The method of embodiment 103, wherein the pCO₂ modulation comprises establishing a high pCO₂ culture.
106. The method of embodiment 105, wherein the pCO₂ is about 20 mmHg to about 250 mmHg; about 20 mmHg to about 250 mmHg; about 20 mmHg to about 150 mmHg; or about 30 mmHg to about 150 mmHg.
107. The method of embodiment 103, wherein the pCO₂ is modulation comprises establishing a low pCO₂ culture.
108. The method of embodiment 107, wherein the pCO₂ is about 10 mmHg to about 100 mmHg; 10 mmHg to about 80 mmHg; about 20 mmHg to about 70 mmHg; or about 30 mmHg to about 60 mmHg.
109. The method of embodiment 103, wherein the pCO₂ modulation occurs at day 0 of the culture.
110. The method of embodiment 103, wherein the pCO₂ modulation occurs for: about the majority of the cell culture; about the first 5 days; about the first 7 days; or about the first 10 days.
111. The method of embodiment 103, wherein the pCO₂ modulation occurs for: about the majority of the production culture; about the first 5 days; about the first 7 days; or about the first 10 days.
112. The method of any one of embodiments 78-111, wherein the duration of the pre-inoculation cell culture media hold is about 0 hrs to about 120 hrs; 0 hrs to about 72 hrs; about 0 hrs to about 48 hrs; or about 0 hrs to about 24 hrs.
113. The method of embodiment 112, wherein the temperature of the media during the pre-inoculation cell culture media hold is about 25° C. to about 39° C.; about 30° C. to about 39° C.; about 35° C. to about 39° C.; or about 36° C. to about 39° C.
114. The method of any one of embodiments 78-113, wherein the duration of the cell culture is about 0 days to about 150 days; about 0 days to about 15 days; about 0 days to about 12 days; 0 days to about 7 days; or about 0 days to about 5 days.
115. The method of any one of embodiments 78-114, wherein the Na+ concentration is about 0 mM to about 300 mM; is about 20 mM to about 200 mM; about 30 mM to about 150 mM; or about 40 mM to about 130 mM.
116. The method of embodiments 115, wherein the modulation of the Na+ concentration comprises supplementing the cell culture with Na compounds including but not limited to: Na₂CO₃, NaHCO₃, NaOH, NaCl, or combinations thereof
117. The method of any one of embodiments 78-116, wherein the osmolality of is about 250 mOsm/kg to about 550 mOsm/kg; about 300 mOsm/kg to about 450 mOsm/kg; or about 325 mOsm/kg to about 425 mOsm/kg.
118. The method of embodiment 117, wherein the modulation of the osmolality comprises supplementing the cell culture with an osmolality-modulating media component.
119. The method of embodiment 118, wherein the osmolality-modulating media component is NaCl, KCl, sorbitol, an osmoprotectant, or combinations thereof.
120. The method of any one of embodiments 787-119, wherein the galactose concentration is about 0 mM to about 60 mM or about 0 mM to about 50 mM.
121. The method of any one of embodiments 78-119, wherein the fucose concentration is about 0 mM to about 60 mM; 0 mM to about 40 mM; about 0 mM to about 20 mM; or about 0 mM to about 10 mM.
122. The method of any one of embodiments 78-119, wherein the cell culture temperature is about 29° C. to about 39° C.; about 30° C. to about 39° C.; about 31° C. to about 38° C.; or about 34° C. to about 38° C.
123. Use of the medium any one of embodiments 78-121 in a eukaryotic cell fermentation process for the production of a recombinant protein.
124. The use of the medium of embodiment 123, wherein the recombinant protein is an antibody or antibody fragment, a scFv (single-chain variable fragment), BsDb (bispecific diabody), scBsDb (single-chain bispecific diabody), scBsTaFv (single-chain bispecific tandem variable domain), DNL-(Fab)3 (dock-and-lock trivalent Fab), sdAb (single-domain antibody) and BssdAb (bispecific single-domain antibody).
125. The use of the medium of embodiment 124, wherein the antibody is a chimeric, a humanized or a human antibody.
126. The use of the medium of embodiment 124, wherein the antibody is an anti-CD20 antibody.
127. The use of the medium of embodiment 124, wherein the anti-CD20 antibody is ocrelizumab.
128. The use of the medium of embodiment 124, wherein the antibody or antibody fragment exhibits: a % G0-F (percent afucosylated glycoprotein) between about 0% to about 20%; about 1% to about 15%; about 1% to about 10%; or about 1% to about 8%; a normalized % G0-F between about 0% to about 20%; about 1% to about 15%; about 1% to about 10%; or about 1% to about 8%; and/or a % G0 (percent agalactosylated glycoprotein) between about 40% to about 90%; about 50% to about 90%; about 55% to about 85%; or about 60% to about 80%.
129. The use of the medium of embodiment 124, wherein the eukaryotic cell is an insect, avian, fungal, plant or mammalian cell.
130. The use of the medium of embodiment 129, wherein the fungal cells are yeast, Pichia or any filamentous fungal cells.
131. The use of the medium of embodiment 130, wherein the yeast cells are S. cerevisiae cells.
132. The use of the medium of embodiment 129, wherein the mammalian cells are CHO cells.
133. The use of the medium of any one of embodiments 123-132, wherein the cell culture is in a bioreactor including but not limited to: a single use technology (SUT) bag or bioreactor; a WAVE bioreactor; a stainless steel bioreactor; a flask; a tube and a chamber.
134. The use of the medium of any one of embodiments 123-133, wherein the volume of the cell culture is from 1 mL to 35,000 L.
135. The use of the medium of embodiment 134, wherein the volume of the cell culture is from 1 mL to 10 ml, from 1 mL to 50 ml, from 1 mL to 100 ml, from 1 mL to 200 ml, from 1 mL to 300 ml, from 1 mL to 500 ml, from 1 mL to 1000 ml, from 1 mL to 2000 ml, from 1 mL to 3000 ml, from 1 mL to 4000 ml, from 1 mL to 5000 ml, from 1 mL to 1 L, from 1 mL to 2 L, from 1 mL to 3 L, from 1 mL to 4 L, from 1 mL to 5 L, from 1 mL to 6 L, from 1 mL to 10 L, from 1 mL to 20 L, from 1 mL to 30 L, from 1 mL to 40 L, from 1 mL to 50 L, from 1 mL to 60 L, from 1 mL to 70 L, from 1 mL to 100 L, from 1 mL to 200 L, from 1 mL to 300 L, from 1 mL to 400 L, from 1 mL to 500 L, from 1 mL to 1000 L, from 1 mL to 2000 L, from 1 mL to 3000 L, from 1 mL to 4000 L, from 1 mL to 5000 L, from 1 mL to 10,000 L, from 1 mL to 20,000 L, from 1 mL to 30,000 L, from 1 mL to 30,000 L, from 1 mL to 35,000 L.
136. A cell culture composition comprising, a host cell engineered to express a glycoprotein of interest; and a cell culture and/or cell culture media modulated to target one or more predetermined parameter selected from: the Mn concentration in a high partial pressure CO₂ (pCO₂) culture from about 1 nM to about 20000 nM; the Mn concentration in a low pCO₂ culture from about 1 nM to about 30000 nM; the pCO₂ from about 10 mmHg to about 250 mmHg; the pre-inoculation cell culture media hold duration from about 0 hrs to about 120 hrs; the cell culture duration from about 0 days to about 150 days; the Na+ concentration from about 0 mM to about 300 mM; the osmolality from about 250 mOsm/kg to about 550 mOsm/kg; the galactose concentration from about 0 mM to about 60 mM; the fucose concentration from about 0 mM to about 60 mM; and the cultivation temperature from about 29° C. to about 39° C.
137. The composition of embodiment 136, wherein the cell culture environment is in a bioreactor.
138. The composition of any one of embodiments 136-137, wherein the glycoprotein of interest is an antibody or antibody fragment.
139. The composition of embodiment 138, wherein the antibody or antibody fragment exhibits: a % G0-F (percent afucosylated glycoprotein) between about 0% to about 20%; about 1% to about 15%; about 1% to about 10%; or about 1% to about 8%; or, a normalized % G0-F between about 0% to about 20%; about 1% to about 15%; about 1% to about 10%; or about 1% to about 8%; and/or, a % G0 (percent agalactosylated glycoprotein) between about 40% to about 90%; about 50% to about 90%; about 55% to about 85%; or about 60% to about 80%.
140. The composition of embodiment 138, wherein the glycosylation of the antibody or antibody fragment is modulated to achieve: an increased afucosylation (e.g., G0-F (afucosylated G0)), while decreasing agalactosylation (e.g., G0 (fucosylated, agalactosylated G0)); or, a decreased afucosylation (e.g., G0-F), while increasing agalactosylation (e.g., G0); or, an increased or decreased afucosylation (e.g., G0-F) without impacting agalactosylation (e.g., G0); or, an increased or decreased agalactosylation (e.g., G0) without impacting afucosylation (e.g., G0-F).
141. The composition of embodiment 136, wherein the Mn concentration is from about 1 nM to about 30000 nM and the duration of the pre-inoculation cell culture media hold is from about 0 hrs to about 120 hrs.
142. The composition of embodiment 136, wherein the pCO₂ is from about 10 mmHg to about 250 mmHg, the Na+ concentration from about 0 mM to about 300 mM, and the duration of the pre-inoculation cell culture media hold is from about 0 hrs to about 120 hrs.
143. The composition of embodiment 136, wherein the Mn concentration is from about 1 nM to about 30000 nM, the pCO₂ is from about 10 mmHg to about 250 mmHg, and the Na+ concentration is from about 0 mM to about 300 mM.
144. The composition of embodiment 136, wherein the Mn concentration is from about 1 nM to about 30000 nM, the pCO₂ is from about 10 mmHg to about 250 mmHg, the Na+ concentration is from about 0 mM to about 300 mM, and the duration of the pre-inoculation cell culture media hold is from about 0 hrs to about 120 hrs.
145. The composition of embodiment 136, wherein the pCO₂ is from about 10 mmHg to about 250 mmHg and the Na+ concentration is from about 0 mM to about 300 mM.
146. The composition of embodiment 136, wherein the osmolality is from about 250 mOsm/kg to about 550 mOsm/kg and the pCO₂ is from about 10 mmHg to about 250 mmHg.
147. The composition of embodiment 136, wherein the pCO₂ is from about 10 mmHg to about 250 mmHg, the Mn concentration is from about 1 nM to about 30000 nM, the duration of the cell culture is from about 0 days to about 150 days, and the duration of the pre-inoculation cell culture media hold is from about 0 hrs to about 120 hrs
148. The composition of embodiment 136, wherein the Mn concentration is from about 1 nM to about 30000 nM and the galactose concentration is from about 0 mM to about 60 mM.
149. The composition of embodiment 136, wherein the fucose concentration is from about 0 mM to about 60 mM and the Mn concentration is from about 1 nM to about 30000 nM.
150. The composition of embodiment 136, wherein the fucose concentration is from about 0 mM to about 60 mM and the pCO₂ is from about 10 mmHg to about 250 mmHg.
151. The composition of embodiment 136, wherein the fucose concentration is from about 0 mM to about 60 mM, the Mn concentration is from about 1 nM to about 30000 nM, and the pCO₂ is from about 10 mmHg to about 250 mmHg.
152. The composition of embodiment 136, wherein the fucose concentration is from about 0 mM to about 60 mM and the cell culture temperature is about 29° C. to about 39° C.
153. The composition of embodiment 136, wherein the fucose concentration is from about 0 mM to about 60 mM and the duration of the cell culture is from about 0 days to about 150 days.
154. The composition of any one of embodiments 136-153, Mn concentration is from about 1 nM to about 20000 nM in a high pCO₂ culture; from about 1 nM to about 10000 nM, from about 1 nM to about 5000 nM, from about 1 nM to about 4000 nM, from about 1 nM to about 3000 nM, from about 1 nM to about 2000 nM, from about 1 nM to about 1000 nM in a high pCO₂ culture; from about 1 nM to about 500 nM, from about 1 nM to about 100 nM, from about 1 nM to about 50 nM, from about 1 nM to about 20 nM, from about 20 nM to about 2000 nM, from about 20 nM to about 3000 nM, from about 20 nM to about 10000 nM, from about 20 nM to about 20,000 nM, from about 20 nM to about 300 nM, about 30 nM to about 110 nM in a high pCO₂ culture.
155. The method of any one of embodiments 136-153, wherein the Mn concentration is about 1 nM to about 30000 nM in a low pCO₂ culture; from about 1 nM to about 20000 nM; from about 1 nM to about 10000 nM, from about 1 nM to about 5000 nM, from about 1 nM to about 4000 nM, from about 1 nM to about 3000 nM, from about 1 nM to about 2000 nM, from about 1 nM to about 1000 nM; from about 1 nM to about 500 nM, from about 1 nM to about 100 nM, from about 1 nM to about 50 nM, from about 1 nM to about 20 nM, from about 20 nM to about 100 nM, about 20 nM to about 300 nM, from about 20 nM to about 500 nM, from about 20 nM to about 1000 nM, from about 20 nM to about 2000 nM, from about 20 nM to about 3000 nM, from about 20 nM to about 5000 nM, from about 20 nM to about 10000 nM, from about 20 nM to about 20000 nM, or about 30 nM to about 110 nM in a low pCO₂ culture.
156. The composition of embodiment 154 or embodiment 155, wherein modulation of the Mn concentration comprises determining the Mn content in cell culture raw materials and selecting raw material lots to modulate the Mn concentration.
157. The composition of embodiment 154 or embodiment 155, wherein modulation of the Mn concentration comprises (i) controlling materials that come into contract with cell culture media or cell culture; or (ii) accounting for the concentration of leached Mn in cell culture media or during cell culture; or a combination of (i) and (ii) to modulate the Mn concentration.
158. The composition of embodiment 157, wherein the leached Mn is produced by contact of the cell culture and/or cell culture media with: (i) a filter; (ii) a media preparation, hold, or culture vessel; or (iii) combinations of (i) and (ii).
159. The composition of embodiment 158, wherein the filter includes but is not limited to: a depth filter, a column, a membrane and a disc.
160. The composition of embodiment 158, wherein the filter material includes but is not limited to: diatomaceous earth, hollow fibers or a resin.
161. The composition of embodiment 154 or embodiment 155, wherein Mn is supplemented as a component of a cell culture media.
162. The composition of embodiment 161, wherein the cell culture media is a feed media, hydrolysate, or additive.
163. The composition of embodiment 162, wherein the feed media, hydrolysate, or additive comprises Mn.
164. The composition of embodiment 162, wherein the feed media or additive consists essentially of Mn.
165. The composition of embodiment 154 or embodiment 155, wherein Mn is supplemented during the production stage of the cell culture.
166. The composition of embodiment 154 or embodiment 155, wherein the Mn is supplemented prior to the production stage of the cell culture.
167. The composition of embodiment 154 or embodiment 155, wherein the Mn is supplemented based on a pre-defined schedule or criteria.
168. The composition of embodiment 154 or embodiment 155, wherein the Mn is supplemented as a bolus, as an intermittent supplement, as a continuous supplement, as a semi-continuous supplement, as a feedback loop-based supplement, or as a combination of one or more of thereof.
169. The composition of embodiment 154 or embodiment 155, wherein the modulation of the Mn concentration comprises employing a cell culture media pH of about 6.1 to about 7.3; or about 6.3 to about 7.3 prior to HTST heat treatment.
170. The composition of any one of embodiments 136-153, wherein modulation of the glycosylation pattern of the glycoprotein of interest comprises modulating the pCO₂.
171. The composition of embodiment 170, wherein the cell culture or cell culture media is in a bioreactor and where modulation of pCO₂ is achieved by modulating: the bioreactor working volume; the bioreactor gas sparging strategy; the bioreactor agitation strategy; the bioreactor feed strategy; the bioreactor perfusion strategy; the bioreactor media exchange strategy; or an any combination thereof
172. The composition of embodiment 170, wherein the pCO₂ modulation comprises establishing a high pCO₂ culture.
173. The composition of embodiment 172, wherein the pCO₂ is about 20 mmHg to about 250 mmHg; about 20 mmHg to about 250 mmHg; about 20 mmHg to about 150 mmHg; or about 30 mmHg to about 150 mmHg.
174. The composition of embodiment 170, wherein the pCO₂ is modulation comprises establishing a low pCO₂ culture.
175. The composition of embodiment 174, wherein the pCO₂ is about 10 mmHg to about 100 mmHg; 10 mmHg to about 80 mmHg; about 20 mmHg to about 70 mmHg; or about 30 mmHg to about 60 mmHg.
176. The composition of embodiment 170, wherein the pCO₂ modulation occurs at day 0 of the culture.
177. The composition of embodiment 170, wherein the pCO₂ modulation occurs for: about the majority of the cell culture; about the first 5 days; about the first 7 days; or about the first 10 days.
178. The composition of embodiment 170, wherein the pCO₂ modulation occurs for: about the majority of the production culture; about the first 5 days; about the first 7 days; or about the first 10 days.
179. The composition of any one of embodiments 1136-153, wherein modulation of the glycosylation pattern of the glycoprotein of interest comprises modulating the duration of the pre-inoculation cell culture media hold, wherein the duration of the pre-inoculation cell culture media hold is about 0 hrs to about 120 hrs; 0 hrs to about 72 hrs; about 0 hrs to about 48 hrs; or about 0 hrs to about 24 hrs.
180. The composition of embodiment 179, wherein the temperature of the media during the pre-inoculation cell culture media hold is about 25° C. to about 39° C.; about 30° C. to about 39° C.; about 35° C. to about 39° C.; or about 36° C. to about 39° C.
181. The composition of any one of embodiments 136-153, wherein modulation of the glycosylation pattern of the glycoprotein of interest comprises modulating the duration of the cell culture, wherein the duration of the cell culture is about 0 days to about 150 days; about 0 days to about 15 days; about 0 days to about 12 days; 0 days to about 7 days; or about 0 days to about 5 days.
182. The composition of any one of embodiments 136-153, wherein modulation of the glycosylation pattern of the glycoprotein of interest comprises modulating the Na+ concentration, wherein the Na+ concentration is about 0 mM to about 300 mM; is about 20 mM to about 200 mM; about 30 mM to about 150 mM; or about 40 mM to about 130 mM.
183. The composition of embodiments 182, wherein the modulation of the Na+ concentration comprises supplementing the cell culture with Na compounds including but not limited to: Na₂CO₃, NaHCO₃, NaOH, NaCl, or combinations thereof
184. The composition of embodiment 182, wherein Na+ is supplemented during the production stage of the cell culture.
185. The composition of embodiment 182, wherein the Na+ is supplemented prior to the production stage of the cell culture.
186. The composition of embodiment 182, wherein the Na+ is supplemented based on a pre-defined schedule or criteria.
187. The composition of embodiment 182, wherein the Na+ is supplemented as a bolus, as an intermittent supplement, as a continuous supplement, as a semi-continuous supplement, as a feedback loop-based supplement, or as a combination of one or more of thereof
188. The composition of any one of embodiments 136-153, wherein modulation of the glycosylation pattern of the glycoprotein of interest comprises modulating the osmolality, wherein the osmolality of is about 250 mOsm/kg to about 550 mOsm/kg; about 300 mOsm/kg to about 450 mOsm/kg; or about 325 mOsm/kg to about 425 mOsm/kg.
189. The composition of embodiment 188, wherein the modulation of the osmolality comprises supplementing the cell culture with an osmolality-modulating media component.
190. The composition of embodiment 189, wherein the osmolality-modulating media component is NaCl, KCl, sorbitol, an osmoprotectant, or combinations thereof.
191. The composition of embodiment 189, wherein the osmolality-modulating media component is supplemented during the production stage of the cell culture.
192. The composition of embodiment 189, wherein the osmolality-modulating media component is supplemented prior to the production stage of the cell culture.
193. The composition of embodiment 189, wherein the osmolality-modulating media component is supplemented based on a pre-defined schedule or criteria.
194. The composition of embodiment 189, wherein the osmolality-modulating media component is supplemented as a bolus, as an intermittent supplement, as a continuous supplement, as a semi-continuous supplement, as a feedback loop-based supplement, or as a combination of one or more of thereof
195. The composition of any one of embodiments 136-153, wherein modulation of the glycosylation pattern of the glycoprotein of interest comprises modulating the galactose concentration, wherein the galactose concentration is about 0 mM to about 60 mM or about 0 mM to about 50 mM.
196. The composition of embodiment 195, wherein galactose is supplemented as a component of a cell culture media.
197. The composition of embodiment 196, wherein the cell culture media is a feed media, hydrolysate, or additive.
198. The composition of embodiment 197, wherein the feed media, hydrolysate, or additive comprises galactose.
199. The composition of embodiment 197, wherein the feed media or additive consists essentially of galactose.
200. The composition of embodiment 196, wherein galactose is supplemented during the production stage of the cell culture.
201. The composition of embodiment 196, wherein the galactose is supplemented prior to the production stage of the cell culture.
202. The composition of embodiment 196, wherein the galactose is supplemented based on a pre-defined schedule or criteria.
203. The composition of embodiment 196, wherein the galactose is supplemented as a bolus, as an intermittent supplement, as a continuous supplement, as a semi-continuous supplement, as a feedback loop-based supplement, or as a combination of one or more of thereof
204. The composition of any one of embodiments 136-153, wherein modulation of the glycosylation pattern of the glycoprotein of interest comprises modulating the fucose concentration, wherein the fucose concentration is about 0 mM to about 60 mM; 0 mM to about 40 mM; about 0 mM to about 20 mM; or about 0 mM to about 10 mM.
205. The composition of embodiments 204, wherein fucose is supplemented as a component of a cell culture media.
206. The composition of embodiment 205, wherein the cell culture media is a feed media, hydrolysate, or additive.
207. The composition of embodiment 206, wherein the feed media, hydrolysate, or additive comprises fucose.
208. The composition of embodiment 206, wherein the feed media or additive consists essentially of fucose.
209. The composition of embodiment 205, wherein fucose is supplemented during the production stage of the cell culture.
210. The composition of embodiment 205, wherein the fucose is supplemented prior to the production stage of the cell culture.
211. The composition of embodiment 205, wherein the fucose is supplemented based on a pre-defined schedule or criteria.
212. The composition of embodiment 205, wherein the fucose is supplemented as a bolus, as an intermittent supplement, as a continuous supplement, as a semi-continuous supplement, as a feedback loop-based supplement, or as a combination of one or more of thereof
213. The composition of any one of embodiments 136-153, wherein modulation of the glycosylation pattern of the glycoprotein of interest comprises modulating the cell culture temperature, wherein the cell culture temperature is about 29° C. to about 39° C.; about 30° C. to about 39° C.; about 31° C. to about 38° C.; or about 34° C. to about 38° C.
214. The composition of embodiment 213, wherein the cell culture temperature is modulated during the production stage of the cell culture.
215. The composition of embodiment 213, wherein the cell culture temperature is
modulated prior the production stage of the cell culture.
216. The composition of embodiment 213, wherein the cell culture temperature is modulated based on a pre-defined schedule or criteria.
217. The composition of any one of embodiments 136-153, wherein the cell culture comprises eukaryotic cells.
218. The composition of embodiment 217, eukaryotic cells are fungal cells or mammalian cells.
219. The composition of embodiment 218, wherein the fungal cells are yeast cells.
220. The composition of embodiment 219, wherein the yeast cells are S. cerevisiae cells.
221. The composition of embodiment 218, wherein the mammalian cells are CHO cells.
222. The composition of any one of embodiments 136-221, wherein the cell culture is in a bioreactor including but not limited to: a single use technology (SUT) bag or bioreactor; a WAVE bioreactor; a stainless steel bioreactor; a flask; a tube and a chamber.
223. The composition of any one of embodiments 136-222, wherein the volume of the cell culture is from 1 mL to 35,000 L.
224. A method for producing a glycoprotein of interest in a cell culture, comprising: subjecting a cell culture medium suitable for cultivating a eukaryotic cell to the method according to any one of embodiments 1-75, inoculating the modulated cell culture medium with the eukaryotic cell that expresses the recombinant protein; cultivating the eukaryotic cell so that the recombinant protein is expressed.
225. The method for producing a glycoprotein of interest in a cell culture of embodiment 224, wherein the cell culture is in a bioreactor.
226. The method for producing a glycoprotein of interest in a cell culture of embodiment 224, wherein the low pCO₂ condition is from about 10 to about 100 mmHg, and the high pCO₂ condition is from about 20 to about 250 mmHg.
227. The method for producing a glycoprotein of interest in a cell culture of embodiment 3, wherein the duration of pCO₂ modulation covers at least the first half of the cell culture duration.
228. The method of any of embodiments 224-227, wherein the glycoprotein of interest is a recombinant protein.
229. The method of embodiment 228, wherein the recombinant protein is an antibody or antibody fragment, a scFv (single-chain variable fragment), BsDb (bispecific diabody), scBsDb (single-chain bispecific diabody), scBsTaFv (single-chain bispecific tandem variable domain), DNL-(Fab)3 (dock-and-lock trivalent Fab), sdAb (single-domain antibody) and BssdAb (bispecific single-domain antibody).
230. The method of embodiment 229, wherein the antibody is a chimeric, a humanized or a human antibody.
231. The method of embodiment 229, wherein the antibody is an anti-CD20 antibody.
232. The method of embodiment 231, wherein the anti-CD20 antibody is ocrelizumab.
233. The method of any one of embodiments 224-232, wherein the antibody or antibody fragment exhibits: a % G0-F (percent afucosylated glycoprotein) between about 0% to about 20%; about 1% to about 15%; about 1% to about 10%; or about 1% to about 8%; or, a normalized % G0-F between about 0% to about 20%; about 1% to about 15%; about 1% to about 10%; or about 1% to about 8%; and/or, a % G0 (percent agalactosylated glycoprotein) between about 40% to about 90%; about 50% to about 90%; about 55% to about 85%; or about 60% to about 80%.
234. The method of any one of embodiments 224-233, wherein the glycosylation is modulated to achieve: an increased afucosylation (e.g., G0-F (afucosylated G0)), while decreasing agalactosylation (e.g., G0 (fucosylated, agalactosylated G0)); or, a decreased afucosylation (e.g., G0-F), while increasing agalactosylation (e.g., G0); or, an increased or decreased afucosylation (e.g., G0-F) without impacting agalactosylation (e.g., G0); or, an increased or decreased agalactosylation (e.g., G0) without impacting afucosylation (e.g., G0-F).
235. A method of modulating the glycosylation of a glycoprotein of interest, the method comprising: assaying cell culture media to determine if the manganese concentration of the cell culture media falls within a targeted range; and culture a host cell engineered to express the glycoprotein of interest in the cell culture media falling within the targeted range; wherein the glycosylation of glycoproteins of interest is modulated as compared to the glycosylation of glycoproteins of interest expressed by the host cell in culture media falling outside the targeted range of manganese concentrations.
236. The method of embodiment 235, wherein the glycoprotein of interest is an antibody.
237. The method of embodiments 236, wherein the antibody is a chimeric antibody, a humanized antibody, or a human antibody.
238. The method of any one of embodiments 236-237, wherein the antibody is an anti-CD20 antibody.
239. The method of any one of embodiments 297-298, wherein the anti-CD20 antibody is ocrelizumab.
240. The method of embodiment 235, wherein the host cell is a mammalian cell.
241. The method of any one of embodiments 239-240, wherein the host cell is a Chinese
Hamster Ovary (CHO) cell.
242. The method of embodiment 235, wherein the manganese concentration target range is between about 30 nM and about 110 nM.
243. The method of embodiment 235, wherein the assaying of the cell culture media comprises assaying the manganese concentration of a component of the cell culture media.
244. The method of embodiment 243, wherein the component of the cell culture media is a hydrolysate or a serum.
245. The method of any one of embodiments 235-244, wherein the glycosylation is modulated to achieve an in increased G0-F (afucosylated G0), while decreasing G0 (fucosylated G0).
246. A cell culture composition comprising, a cell culture media assayed to determine if the manganese concentration of the cell culture media falls within a targeted range; and a host cell engineered to express a glycoprotein of interest.
247. The cell culture composition of embodiment 246, wherein composition further comprises the glycoprotein of interest.
248. The cell culture composition of embodiment 247, wherein the glycoprotein is an antibody.
249. The cell culture composition of embodiment 248, wherein the antibody is a chimeric antibody, a humanized antibody, or a human antibody.
250. The cell culture composition of any one of embodiments 248-249, wherein the antibody is an anti-CD20 antibody.
251. The cell culture composition of any one of embodiments 248-249, wherein the anti-CD20 antibody is ocrelizumab.
252. The cell culture composition of embodiment 246, wherein the host cell is a mammalian cell.
253. The cell culture composition of any one of embodiments 252, wherein the host cell is a CHO cell.
254. The cell culture composition of embodiment 246, wherein the manganese concentration target range is between about 30 nM and about 110 nM.
255. A composition comprising a glycoprotein of interest, wherein the preparation comprises: a cell culture media assayed to determine if the manganese concentration of the cell culture media falls within a targeted range; a host cell engineered to express a glycoprotein of interest; and the glycoprotein of interest.
256. The composition of embodiment 255, wherein the glycoprotein is an antibody.
257. The composition of embodiment 256, wherein the antibody is a chimeric antibody, a humanized antibody, or a human antibody.
258. The cell culture composition of any one of embodiments 256-257, wherein the antibody is the antibody is an anti-CD20 antibody.
259. The cell culture composition of any one of embodiments 256-257, wherein the anti-CD20 antibody is ocrelizumab.
260. The cell culture composition of embodiment 255, wherein the host cell is a mammalian cell.
261. The cell culture composition of embodiments 260, wherein the host cell is a CHO cell.
262. A method of modulating the glycosylation of a glycoprotein of interest, the method comprising: supplementing a cell culture media employed in culturing host cells expressing the glycoprotein of interest with between about 10 nM and about 2000 nM manganese under high CO₂ conditions; or supplementing the cell culture supplementing the cell culture media employed in culturing a host cell expressing the glycoprotein of interest with between about 10 nM and bout 3000 nM manganese under low CO₂ conditions; wherein the glycosylation of glycoproteins of interest is modulated as compared to the glycosylation of glycoproteins of interest expressed by the host cell in culture media that has not been so supplemented.
263. The method of embodiment 262, wherein the glycoprotein of interest is an antibody.
264. The method of embodiments 263, wherein the antibody is a chimeric antibody, a humanized antibody, or a human antibody.
265. The method of any one of embodiments 263-264, wherein the antibody is ocrelizumab.
266. The method of embodiment 262, wherein the host cell is a mammalian cell.
267. The method of embodiment 266, wherein the host cell is a CHO cell.
268. The method of any one of embodiments 262-267, wherein the glycosylation is modulated to achieve an in increased G0-F (afucosylated G0), while decreasing G0 (fucosylated G0).
269. A cell culture composition comprising, a cell culture media supplemented with: between about 10 nM and about 2000 nM manganese under high CO₂ conditions; or between about 10 nM and about 3000 nM manganese under low CO₂ conditions; and a host cell engineered to express a glycoprotein of interest.
270. The cell culture composition of embodiment 269, wherein composition further comprises the glycoprotein of interest.
271. The cell culture composition of embodiment 269, wherein the glycoprotein is an antibody.
272. The cell culture composition of embodiment 271, wherein the antibody is a chimeric antibody, a humanized antibody, or a human antibody.
273. The cell culture composition of any one of embodiments 271-272, wherein the antibody is ocrelizumab.
274. The cell culture composition of embodiment 269, wherein the host cell is a mammalian cell.
275. The cell culture composition of embodiment 274, wherein the host cell is a CHO cell.
276. A composition comprising a glycoprotein of interest, wherein the preparation comprises: a manganese supplemented cell culture media wherein the culture is supplemented with between about 10 nM and about 2000 nM manganese under high CO₂ conditions; or between about 10 nM and about 3000 nM manganese under low CO₂ conditions; a host cell engineered to express the glycoprotein of interest; and the glycoprotein of interest.
277. The composition of embodiment 276, wherein the glycoprotein is an antibody.
278. The composition of embodiment 277, wherein the antibody is a chimeric antibody, a humanized antibody, or a human antibody.
279. The cell culture composition of any one of embodiments 276-277, wherein the antibody is ocrelizumab.
280. The cell culture composition of embodiment 276, wherein the host cell is a mammalian cell.
281. The cell culture composition of embodiments 280, wherein the host cell is a CHO cell.
282. A method of modulating the glycosylation of a glycoprotein of interest, the method comprising: exposing cell culture media comprising a pH target of 6.30 to 7.25 to high temperature short time (HTST) heat treatment; and culturing a host cell expressing the glycoprotein of interest in the cell culture media; wherein the glycosylation of the glycoproteins of interest is modulated as compared to the glycosylation of the glycoproteins of interest expressed by the host cell in culture media where the pre-HTST heat treatment pH target is greater than pH 7.25.
283. The method of embodiment 282, wherein the glycoprotein of interest is an antibody.
284. The method of embodiments 283, wherein the antibody is a chimeric antibody, a humanized antibody, or a human antibody.
285. The method of any one of embodiments 283-284, wherein the antibody is ocrelizumab.
286. The method of embodiment 282, wherein the host cell is a mammalian cell.
287. The method of embodiment 286, wherein the host cell is a CHO cell.
288. The method of any one of embodiments 282-287, wherein the glycosylation is modulated to achieve an in increased G0-F (afucosylated G0), while decreasing G0 (fucosylated G0).
289. A cell culture composition comprising, a cell culture media comprising a pH target of about 6.30 to about 7.25 exposed to a HTST heat treatment; and a host cell engineered to express a glycoprotein of interest.
290. The cell culture composition of embodiment 289, wherein composition further comprises the glycoprotein of interest.
291. The cell culture composition of embodiment 290, wherein the glycoprotein is an antibody.
292. The cell culture composition of embodiment 291, wherein the antibody is a chimeric antibody, a humanized antibody, or a human antibody.
293. The cell culture composition of any one of embodiments 291-292, wherein the antibody is ocrelizumab.
294. The cell culture composition of embodiment 293, wherein the host cell is a mammalian cell.
295. The cell culture composition of embodiment 294, wherein the host cell is a CHO cell.
296. A composition comprising a glycoprotein of interest, wherein the preparation comprises: a cell culture media comprising a pH target of about 6.30 to about 7.25 exposed to HTST heat treatment; a host cell engineered to express a glycoprotein of interest; and the glycoprotein of interest.
297. The composition of embodiment 296, wherein the glycoprotein is an antibody.
298. The composition of embodiment 297, wherein the antibody is a chimeric antibody, a humanized antibody, or a human antibody.
299. The cell culture composition of any one of embodiments 297-298, wherein the antibody is ocrelizumab.
300. The cell culture composition of embodiment 296, wherein the host cell is a mammalian cell.
301. The cell culture composition of embodiments 300, wherein the host cell is a CHO cell.
302. A method of modulating the glycosylation of a glycoprotein of interest, the method comprising: culturing a host cell expressing the glycoprotein of interest in a cell culture media where: the cell culture is exposed to high pCO₂, the cell culture is exposed to an extended media hold time, and/or the cell culture comprises an increased Na+ concentration; wherein the glycosylation of the glycoproteins of interest is modulated as compared to the fucosylation of a preparation of glycoproteins of interest expressed by the host cell in culture media exposed to low pCO₂, a shortened media hold time, and/or a reduced Na+ concentration.
303. The method of embodiment 302, wherein the glycoprotein of interest is an antibody.
304. The method of embodiments 303, wherein the antibody is a chimeric antibody, a humanized antibody, or a human antibody.
305. The method of any one of embodiments 303-304, wherein the antibody is ocrelizumab.
306. The method of embodiment 302, wherein the host cell is a mammalian cell.
307. The method of embodiment 306, wherein the host cell is a CHO cell.
308. The method of any one of embodiments 302-307, wherein the glycosylation is modulated to achieve an in increased G0-F (afucosylated G0), while decreasing G0 (fucosylated G0).
309. A cell culture composition comprising, a cell culture media comprising high pCO₂, an extended media hold time, and/or an increased Na+ concentration; and a host cell engineered to express a glycoprotein of interest.
310. The cell culture composition of embodiment 309, wherein composition further comprises the glycoprotein of interest.
311. The cell culture composition of embodiment 310, wherein the glycoprotein is an antibody.
312. The cell culture composition of embodiment 311, wherein the antibody is a chimeric antibody, a humanized antibody, or a human antibody.
313. The cell culture composition of any one of embodiments 311-312, wherein the antibody is ocrelizumab.
314. The cell culture composition of embodiment 309, wherein the host cell is a mammalian cell.
315. The cell culture composition of any one of embodiments 314, wherein the host cell is a CHO cell.
316. A composition comprising a glycoprotein of interest, wherein the preparation comprises: a cell culture media comprising high pCO₂, an extended media hold time, and/or an increased Na+ concentration; a host cell engineered to express a glycoprotein of interest; and the glycoprotein of interest.
317. The composition of embodiment 316, wherein the glycoprotein is an antibody.
318. The composition of embodiment 317, wherein the antibody is a chimeric antibody, a humanized antibody, or a human antibody.
319. The cell culture composition of any one of embodiments 317-318, wherein the antibody is ocrelizumab.
320. The cell culture composition of embodiment 316, wherein the host cell is a mammalian cell.
321. The cell culture composition of embodiments 320, wherein the host cell is a CHO cell.

### EXAMPLES

The following examples are merely illustrative of the presently disclosed subject matter and should not be considered as limitations in any way.

### Example 1: Control of Raw Materials to Modulate Glycosylation

Multiple cell culture factors are known to have the potential to impact glycosylation of monoclonal antibody therapeutics. These factors include process parameters, media treatment, and media components, such as galactose and trace metals. Variation in levels of individual media components may be introduced into mAb cell culture process via the use of complex raw materials such as Proteose Peptone No. 3 (PP3) and Genentech Essential Media (GEM) powder. These sources of variability in raw materials can result in substantial differences in Mn concentration at the start of production cultures (i.e., day 0), as outlined in Table 1.

TABLE 1
Day 0 Mn Levels in Ocrelizumab Cell Culture Processes

| Set No. | Day 0 Mn (nM) |
|---|---|
| 1 | >500 |
| 2 | >500 |
| 3 | 63-174 |
| 4 | 31-62 |
| 5 & 6 | 45-56 |

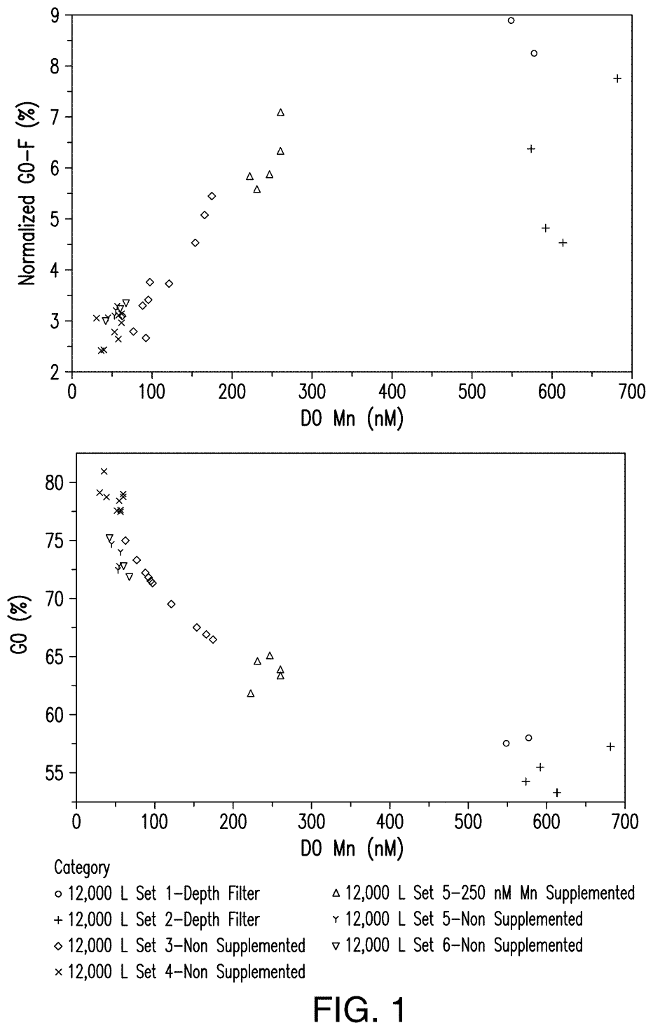

Variation in Mn levels on day 0 of production cultures correlate with variation in % G0 (fucosylated G0) and % G0-F (afucosylated) antibody species (FIG. 1). In FIG. 1, sets 1 and 2 were conducted using media depth filtration, which through Mn leaching from the depth filters. The media depth filtration can contribute significant additional Mn to the production culture medium composition. Day 0 Mn levels was measured by ICP-MS. Set 3 predicted Day 0 Mn levels are based on the equation G0=109.57487-8.34886831n(Mn), which was derived using the sets 1-2 and 4-6 data. Afucosylation is represented by Normalized G0-F=100*[G0-F]/(G0+[G0-F]).

To provide improved control over the Mn contribution from raw materials, either or both of the following strategies can be implemented: (a) test and select PP3 and GEM powder within specified Mn ranges prior to use; and (b) control production culture post-inoculation Day 0 Mn levels within an established acceptable range (e.g., 30 nM to 110 nM).

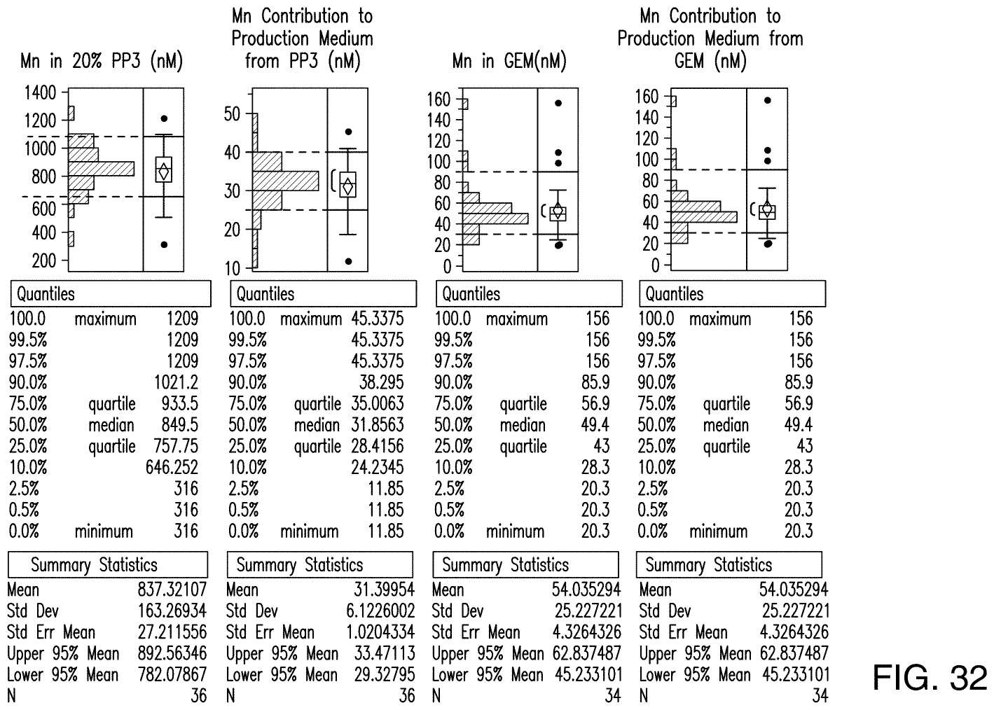

PP3 and GEM powder are selected based on the Mn ranges in Table 2 and FIG. 32. These ranges were established based on Day 0 Mn in the 2016 v1.0 batches and historical data from 36 lots of PP3 and 34 lots of GEM powder, with considerations for losses during media preparation and treatment.

TABLE 2
Raw Material Mn Range

| | | Mn Range |
|---|---|---|
| Raw Material | Test Article | (nM) |
| Genentech Essential | 12.3 g/L | 30-90 |
| Media (GEM) | stock solution | |
| Powder 2 | | |
| Proteose Peptone | 20% stock | 655-1077 |
| No. 3 (PP3) | solution | |

To provide additional assurance of process consistency and control of G0, normalized G0-F and CDC values within specifications, it is possible to control to a narrower range of Day 0 post-inoculation Mn level of 30-110 nM, with an action limit of <30 nM or >110 nM.

### Example 2: Manganese Supplementation to Modulate Glycosylation

2.1 Introduction

This Example summarizes the impact of manganese supplementation for ocrelizumab and other antibodies. With increasing manganese supplementation an increase in afucosylated (G0-F) and decrease in fucosylated (G0) (agalactosylated) species was observed.

2.2 Evaluation of Manganese Supplementation for Ocrelizumab

Manganese supplementation experiments were performed for ocrelizumab. Manganese (Mn) concentration in the test cases was adjusted by a post-inoculation addition to the ocrelizumab production culture. Concentrations of manganese tested in these studies are listed in Table 3. The manganese concentration listed represents the amount of additional manganese added to the culture based on the post-inoculation volume and does not reflect total manganese concentration on day 0 due to presence of manganese in the control process media. Manganese additions were performed using sterile filtered solutions of 0.05 mM and 0.5 mM manganese sulfate monohydrate and added via septum. Replicates of the controls as well as some test cases were included in each study.

TABLE 3
Manganese Addition:

| | | |
|---|---|---|
| | | 0.5 mM Mn Addition |
| Case | Mn Conc (nM) | Volume (mL/L) |

Manganese Addition Study 1

| | | |
|---|---|---|
| Control | 0 | 0 |
| Mn-40 nM | 40 | 0.8 ^{b} |
| Mn-100 nM | 100 | 1.5 ^{b} |
| Mn-1000 nM | 1000 | 2.0 |

Manganese Addition Study 2

| | | |
|---|---|---|
| Control | 0 | 0 |
| Mn-200 nM | 200 | 4.0 ^{b} |
| Mn-500 nM | 500 | 1.0 |
| Mn-700 nM | 700 | 1.4 |
| Mn-1000 nM | 1000 | 2.0 |
| Mn-1500 nM | 1500 | 3.0 |
| Mn-2000 nM | 2000 | 4.0 |

Manganese Addition Study 3

| | | |
|---|---|---|
| Control | 0 | 0 |
| Mn-500 nM | 500 | 1.0 |
| Mn-700 nM | 700 | 1.4 |
| Mn-1000 nM | 1000 | 2.0 |
| Mn-1500 nM | 1500 | 3.0 |
| Mn-2000 nM | 2000 | 4.0 |
| Mn-3000 nM | 3000 | 6.0 |

^{a} Manganese concentrations evaluated represent additional manganese to the ocrelizumab production culture process. A low level of manganese is present in the control media;
^{b} 0.05 mM Manganese sulfate solution used for these cases

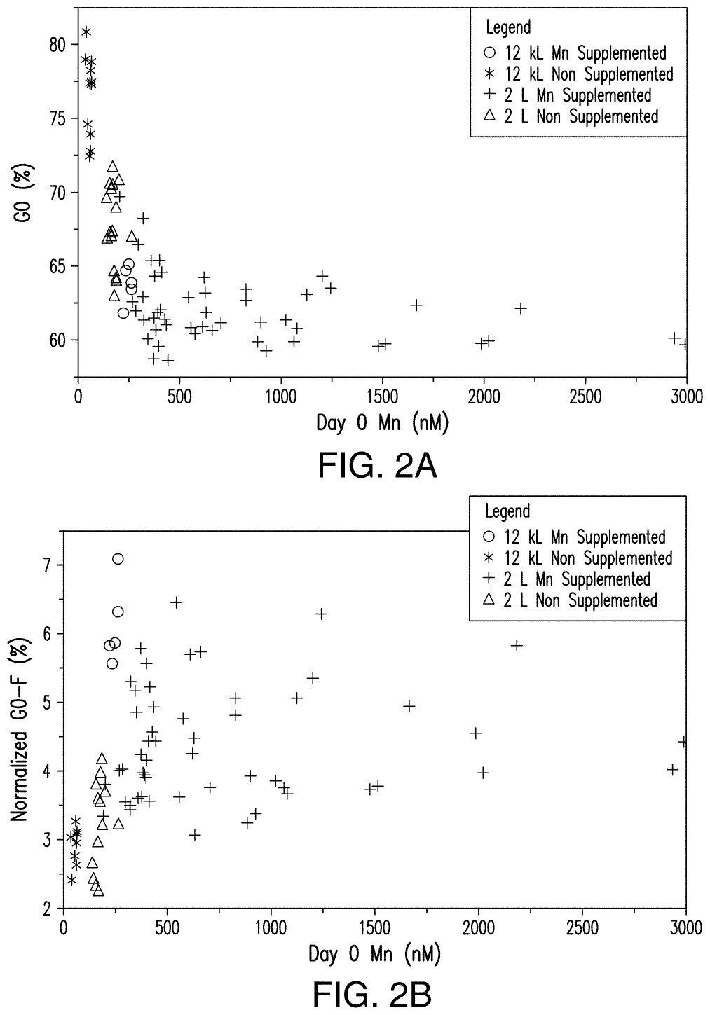

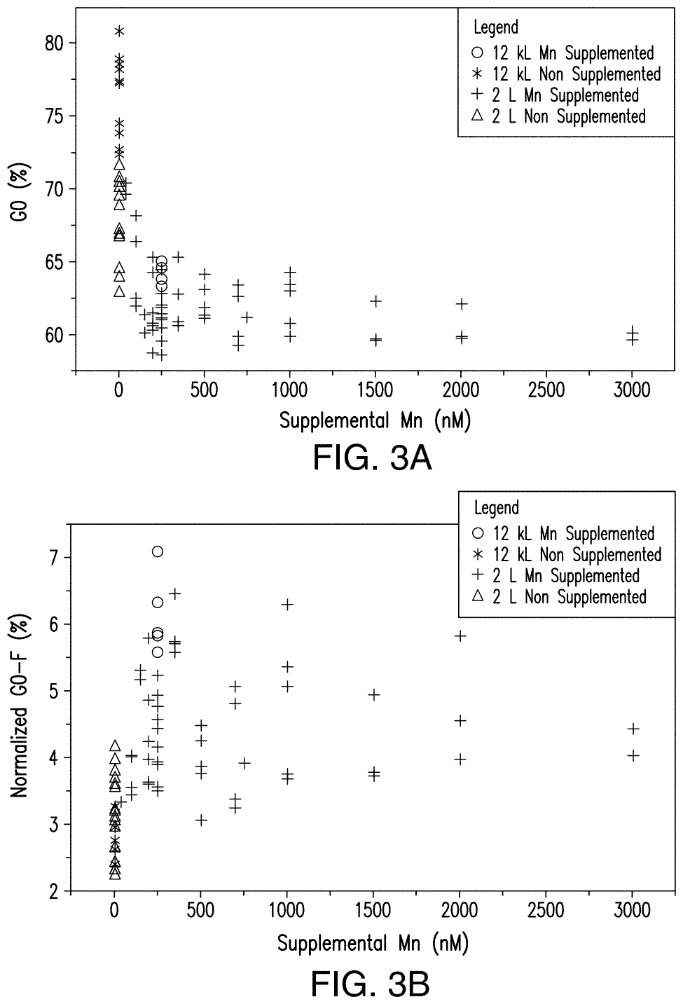

FIG. 2 shows the correlation between day 0 manganese concentration in cell cultures and afucosylation (normalized G0-F) and agalactosylation (G0). FIG. 3 shows the impact of manganese supplementation on ocrelizumab afucosylated (normalized G0-F) and fucosylated, agalactosylated (G0) species. As the concentration of manganese increases, ocrelizumab G0-F increases and G0 decreases. FIGS. 2 and 3 show that the same trend on impact of manganese on afucosylation and agalactosylation across bioreactor scales, thereby demonstrating the scalability of the findings at the small scale (2 L). In particular, at both 2 L and 12 kL bioreactor scales, increased normalized G0-F and decreased G0 were observed in Mn supplemented cultures compared with non-supplemented cultures (with no Mn addition).

2.2 Evaluation of Manganese Supplementation and pCO₂ for Ocrelizumab

A manganese titration at 0, 50, 100, 150, 250, 350, 500, 750, 1000, and 2000 nM manganese was performed in both the scale-dependent factor model (high pCO₂ model with 36-hr media hold at 37° C.) and standard 2 L model. Media for this study was high temperature short time (HTST) heat treated with the following HTST conditions: 10 seconds hold at 102° C., back pressure of 15 psig, and cooling to 37° C. post-HTST. All other conditions and parameters were executed at target conditions (i.e., using same set points).

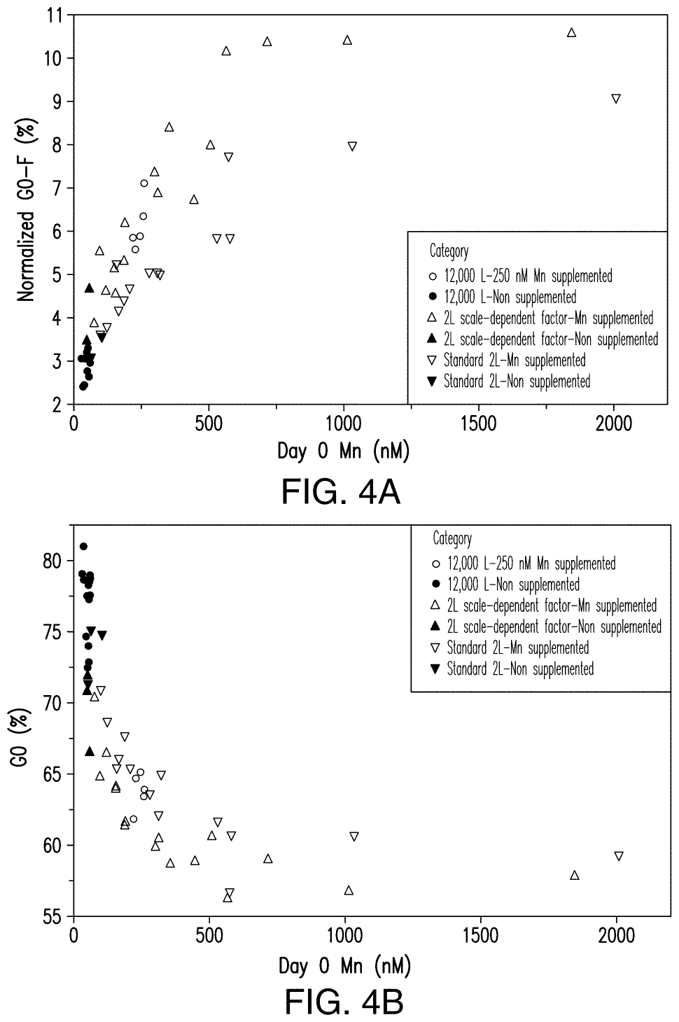

Afucosylated (normalized G0-F) and fucosylated, agalactosylated (G0) results are shown in FIGS. 4 and 5. In the presence of high pCO₂ levels, a larger impact to normalized G0-F and G0 was observed compared to the standard 2 L model (low pCO₂). The trends of this study are consistent with manganese titration study (see 2.2, above). As shown in FIGS. 2-5, bioreactors with various volume (e.g., standard 2 L, 2 L scale-dependent factor, and 12,000 L) can be used for the compositions of cell culture media. Conditions of cell culture media can be adjusted based on the volume of the bioreactor. For example, compositions of cell culture media used in a 2 L bioreactor can be scaled up to be used in a 15,000 L bioreactor. Furthermore, compositions of cell culture media used in a 15,000 L bioreactor can be scaled down to be used in a 2 L bioreactor. Volume of the bioreactor can be between about 1 L and about 20,000 L (e.g., about 1 L, about 1.5 L, about 2 L, about 5 L, about 10 L, about 50 L, about 100 L, about 250 L, about 500 L, about 1000 L, about 2000 L, about 3000 L, about 4000 L, about 5000 L, about 6000 L, about 7000 L, about 8000 L, about 9000 L, about 10,000 L, about 11,000 L, about 12,000 L, about 13,000 L, about 14,000 L, about 15,000 L, about 16,000 L, about 17,000 L, about 18,000 L, about 19,000 L, or about 20,000 L).

2.3 Evaluation of Manganese Concentration for Antibody I

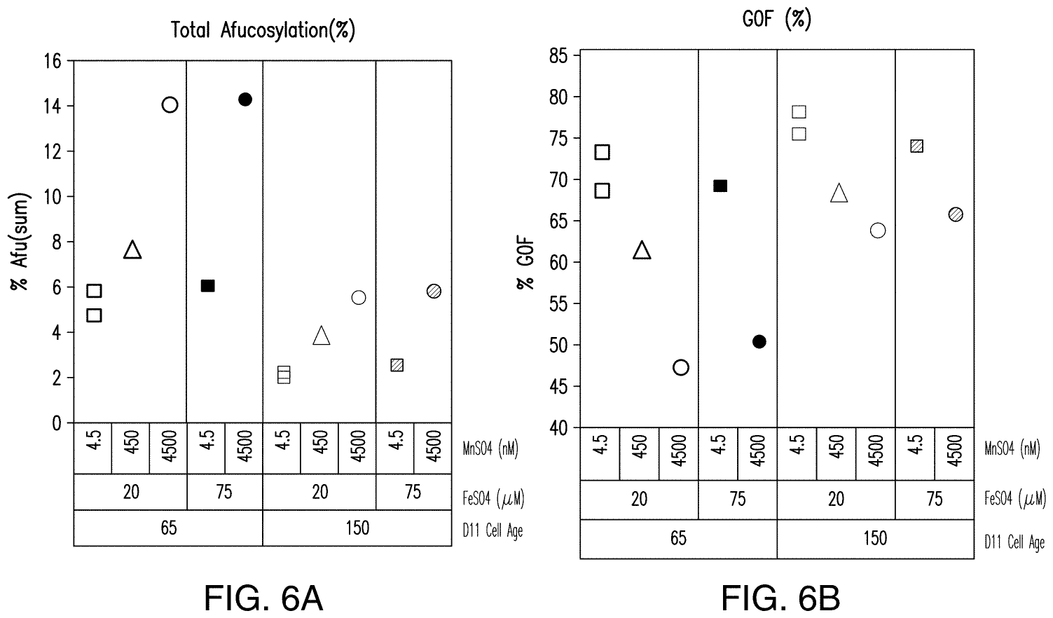

A full factorial DOE (2×2×3) was designed looking at three factors, cell age (66 days vs. 151 days), iron concentration (20 μM vs. 75 μM) and manganese concentration (4.5 nM vs. 450 nM vs. 4500 nM). The goal of the study was to determine the impact of higher manganese levels on glycosylation and the impact of iron concentration for charge variants. Study design is shown in Table 4. Results of G0-F (afucosylation) and G0 (G0F) are shown in FIG. 6. An increase in afucosylation and decrease in G0 was observed with increasing manganese concentration consistent with other antibodies. This result was consistent across all cell ages and iron concentrations indicating the effect of manganese is independent of the other tested parameters.

TABLE 4
Antibody I Cell Age, Manganese, Iron Study

| | | Manganese | Iron |
|---|---|---|---|
| Case | Cell Age (d) | (nM) | (μM) |
| 1 | 66 | 4.5 | 20 |
| 2 | 66 | 4.5 | 75 |
| 3 | 66 | 4500 | 20 |
| 4 | 66 | 4500 | 75 |
| 5 | 151 | 4.5 | 20 |
| 6 | 151 | 4.5 | 75 |
| 7 | 151 | 4500 | 20 |
| 8 | 151 | 4500 | 75 |
| 9 | 66 | 450 | 20 |
| 10 | 151 | 450 | 20 |

2.4 Evaluation of Manganese Supplementation for Antibody II

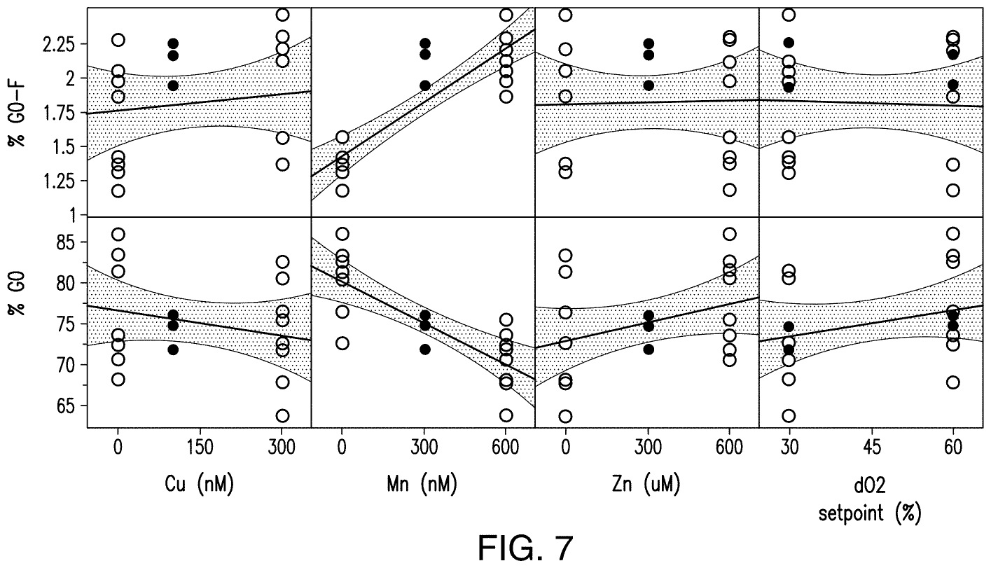

This study design consists of a full factorial DOE that combines three two-level variables: copper addition level, manganese addition level and zinc addition level (Table 5). All proposed process conditions (containing supplemental copper, manganese and zinc at target levels), will be tested in duplicate. Results of the study are shown in FIG. 7. Manganese had the largest effect estimate for both G0 and G0-F. The trends of G0 and G0-F with increasing levels of manganese is consistent with other antibodies.

TABLE 5
Antibody II Copper, Manganese, Zinc
Study Design

| | Copper | Manganese | Zinc |
|---|---|---|---|
| Case | (μM) | (nM) | (μM) |
| 1 | 100 | 300 | 300 |
| 2 | 300 | 600 | 0 |
| 3 | 0 | 0 | 600 |
| 4 | 0 | 0 | 0 |
| 5 | 0 | 600 | 0 |
| 6 | 300 | 0 | 0 |
| 7 | 300 | 0 | 600 |
| 8 | 300 | 600 | 600 |
| 9 | 0 | 600 | 600 |
| 10 | 100 | 300 | 300 |

2.5 Evaluation of Manganese Supplementation for Antibody III

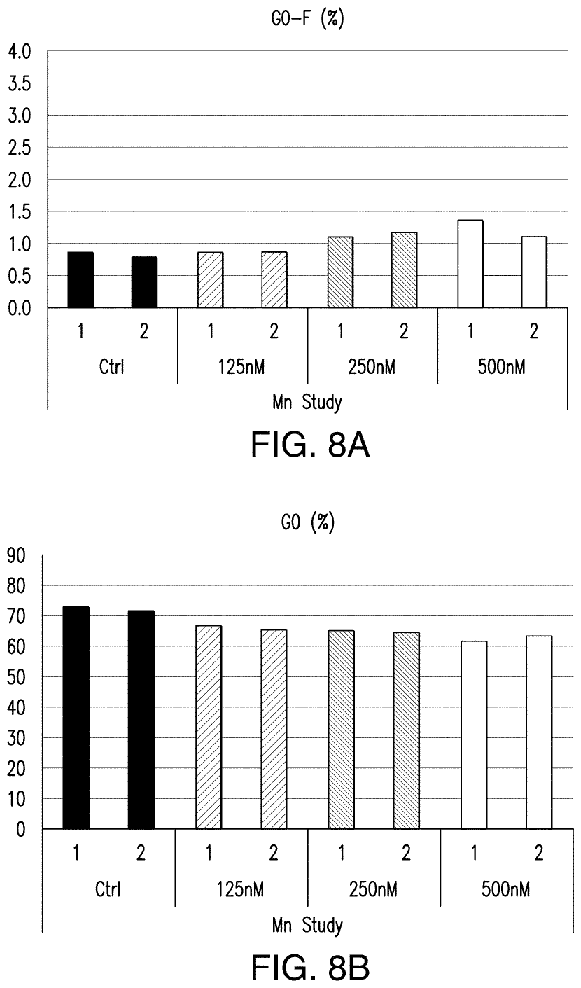

A manganese titration study was performed for Antibody III. 0.5 mM manganese sulfate stock solution was added post production inoculation calculated based on final working volume. The concentrations added in addition to actual measured manganese from the test cases are shown in Table 6. A trace amount of manganese is present in the cell culture media as shown in Case 1 without any additional manganese added to the production culture. While there exists some variability in the measured manganese levels, generally the measured levels via inductively coupled plasma mass spectrometry confirmed that the correct amount of manganese stock solution was added to each bioreactor post production inoculation. All control runs performed within expectations. Additional manganese supplementation had no impact on growth and titer. As expected, fucosylation (G0) (agalactosylation) decreased and afucosylation (G0-F) increased with increased Mn. Results are shown in FIG. 8.

TABLE 6
Antibody III Manganese Titration Study

| | Supplemental | Actual Mn |
|---|---|---|
| | Manganese | Concentration |
| Case | (nM) | (nM) |
| 1 | 0 | 85 |
| 1 | 0 | 82 |
| 2 | 125 | 195 |
| 2 | 125 | 244 |
| 3 | 250 | 365 |
| 3 | 250 | 280 |
| 4 | 500 | 511 |
| 4 | 500 | 507 |

2.6 Evaluation of Manganese Supplementation for Antibody IV and Antibody V

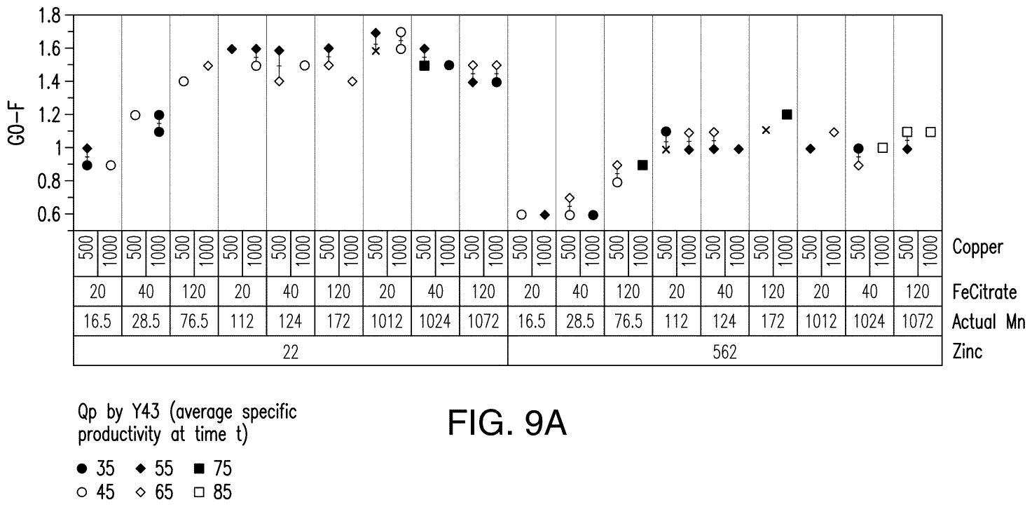

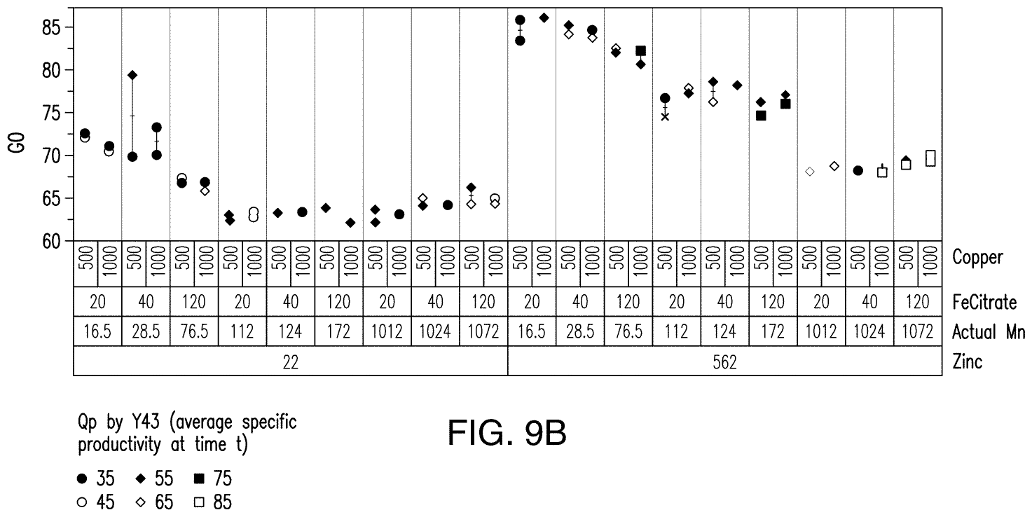

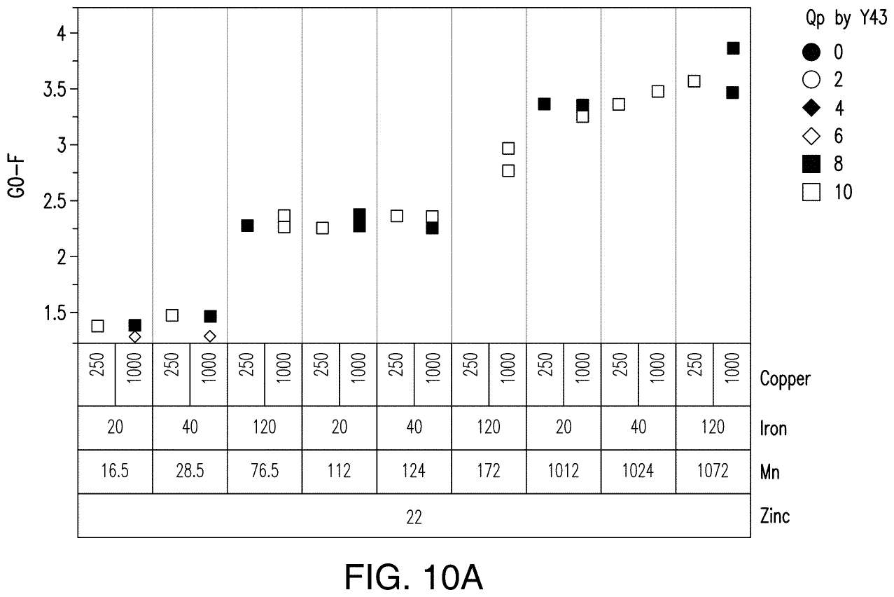

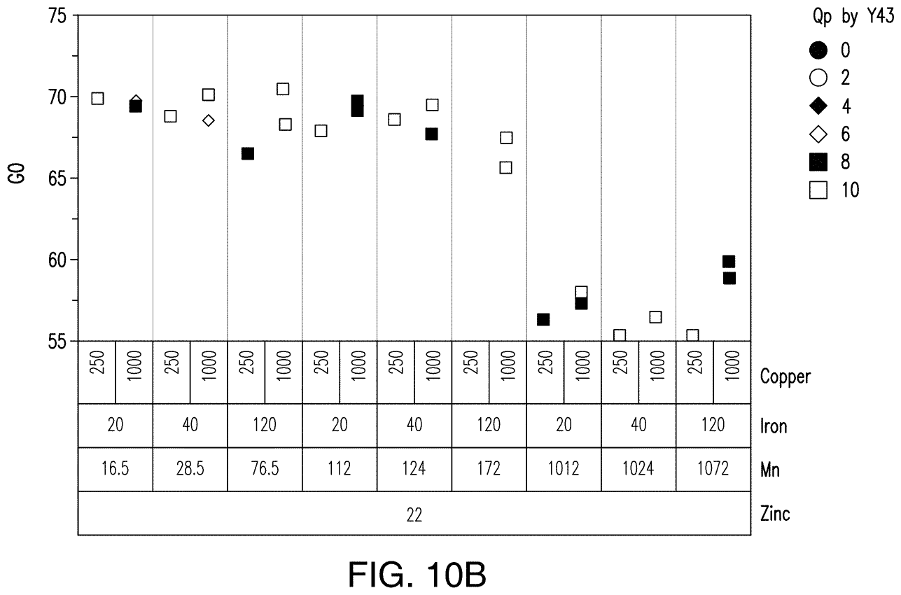

Antibody IV and Antibody V evaluated zinc, manganese, iron, and copper in combination using a full factorial design of experiment to determine their impact to cell culture process performance and product quality of antibody IV and antibody V. Table 7 shows the design of the studies. Results for Antibody IV are shown in FIG. 9 and Antibody V in FIG. 10. FIGS. 9 and 10 display the actual measured manganese. This is different compared to the supplemented amount of manganese as listed in Table 7 due to the presence of manganese in the basal media. With the increase of manganese concentration, G0-F (afucosylated) increases and G0 (fucosylated, agalactosylated) decreases for both Antibody IV and Antibody V. This effect is independent of zinc, iron, and copper concentrations.

TABLE 7
Antibody IV Metal Concentrations

| Variable | Low | | Med | High |
|---|---|---|---|---|
| Zinc | 22 | μM | — | 562 μM |
| Mn | 4.5 | nM | 100 nM | 1000 nM |
| Fe | 20 | μM | 40 μM | 120 μM |
| Cu | 500 | nM | — | 1000 nM |

2.7 Evaluation of Manganese Supplementation Timing for Antibody VI

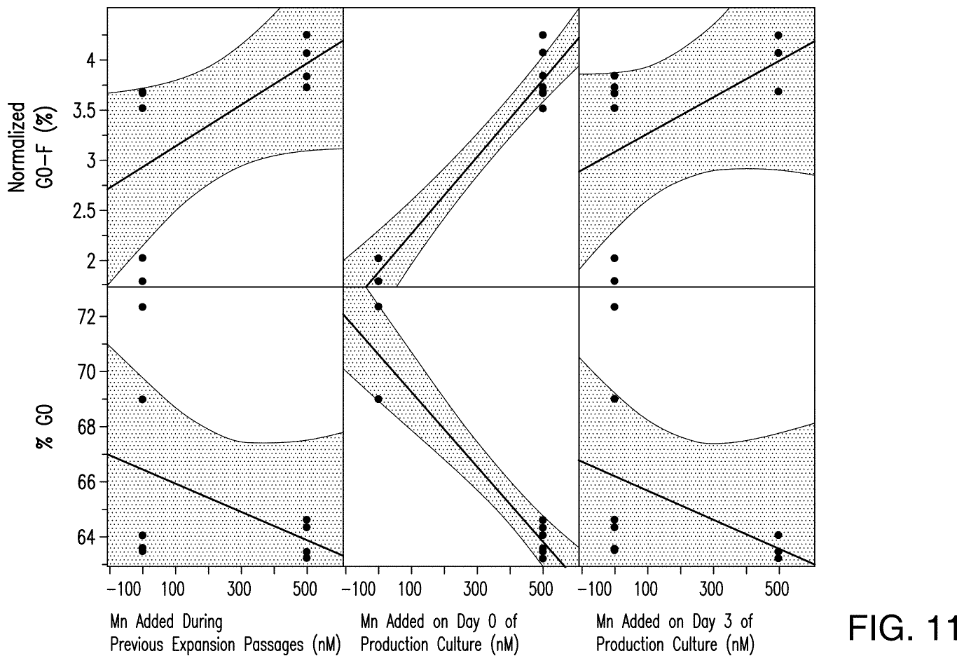

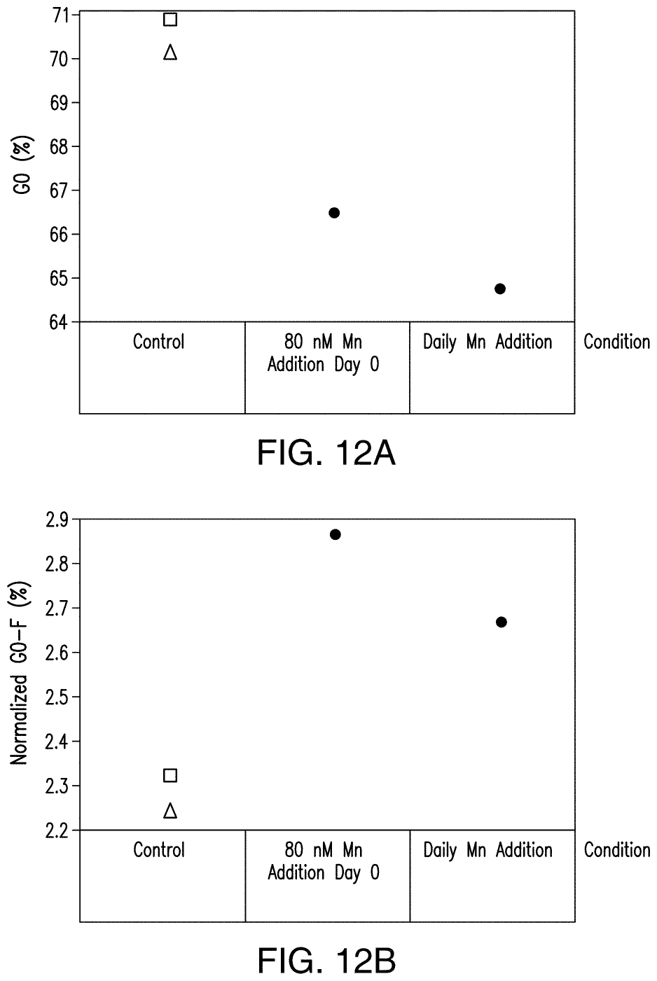

Mn addition timing experiments were performed for Antibody VI to evaluate the impact of different addition timings on glycosylation. In the first study (FIG. 11), cultures were either supplemented with 500 nM manganese (during previous expansion passages, or on day 0 or day 3 of production culture) or they were not supplemented. In the second study (FIG. 12), cultures were either supplemented with 80 nM manganese (on day 0 or daily during production culture) or they were not supplemented. G0 (fucosylated, agalactosylated) decreased and normalized G0-F (afucosylated) increased when Mn was added to the cell culture irrespective of the timing of Mn supplementation.

It will be understood that the foregoing is only illustrative of the principles of the present disclosure, and that various modifications can be made by those skilled in the art without departing from the scope and spirit of the present disclosure. For example, but not by way of limitation, volume of the bioreactor used in the example can be between about 1 L and about 20,000 L (e.g., about 1 L, about 1.5 L, about 2 L, about 5 L, about 10 L, about 50 L, about 100 L, about 250 L, about 500 L, about 1000 L, about 2000 L, about 3000 L, about 4000 L, about 5000 L, about 6000 L, about 7000 L, about 8000 L, about 9000 L, about 10,000 L, about 11,000 L, about 12,000 L, about 13,000 L, about 14,000 L, about 15,000 L, about 16,000 L, about 17,000 L, about 18,000 L, about 19,000 L, or about 20,000 L). Furthermore, bioreactors and their operations can be modified to adjust levels of pCO₂, media hold duration, culture duration, osmolality, Na+, Mn, cultivation temperature, fucose, galactose, or combinations thereof.

### Example 3: Pre-High Temperature Short Time (HTST) Heat Treatment pH Target

3.1 Introduction

This Example summarizes the selection of the pH adjustment target for media prior to high temperature short time (HTST) heat treatment for ocrelizumab (rhuMAb 2H7) production media preparation and supporting experimental results. A lower pre-HTST heat treatment media pH target can reduce media turbidity, associated precipitate formation, HTST heat transfer surface fouling, and filter plugging during HTST operations (See e.g., U.S. Pat. No. 9,493,744).

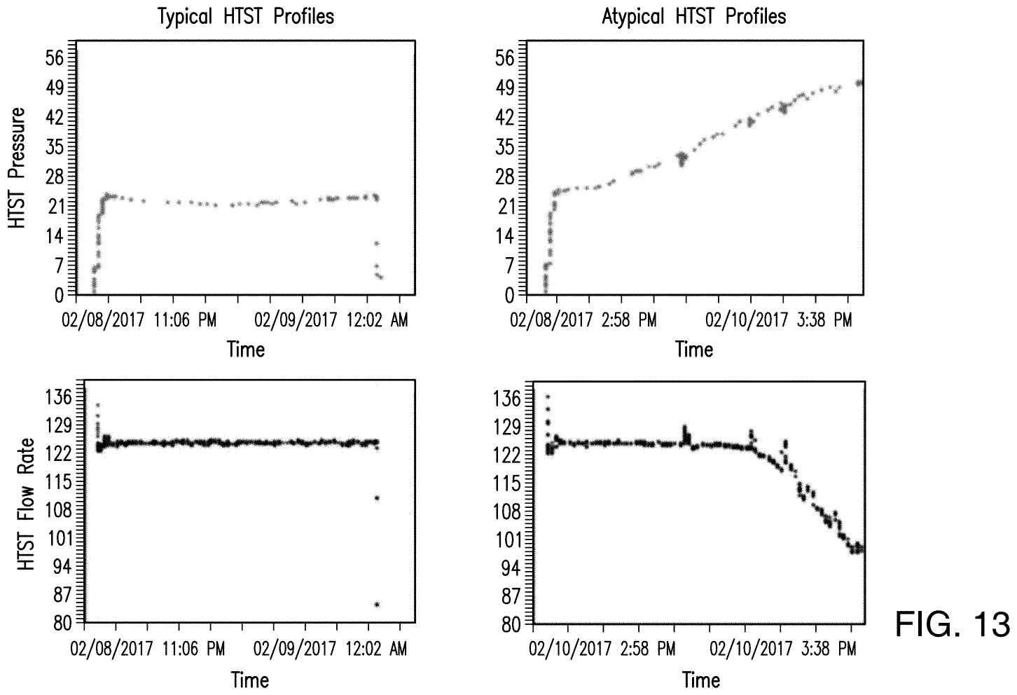

A trend of increasing pressure measured prior to the cooler and decreasing flow rate was observed for several media preparations (FIG. 13). These trends were found to correlate to an increase in manganese (Mn) loss across HTST and filtration operations (Table 9). The increase in HTST pressure (prior to the cooler) and decrease in HTST flow rate in these instances can be attributed to turbidity/precipitate formation and subsequent pressure increase on the medium filters (increased filter plugging). To alleviate this atypical HTST profile in ocrelizumab manufacturing runs and potentially decrease variability in Mn loss across HTST and filtration, a lower pre-HTST pH media adjustment target with a post-HTST pH adjustment was evaluated and implemented for ocrelizumab production media.

TABLE 9
Mn Measurements Across HTST and Filtration Example
HTST Profile Runs All manganese measurements were
performed using an inductively coupled plasma mass
spectrometry (ICP- MS) assay method.

| | | Post-HTST / | Percent Mn Loss | |
|---|---|---|---|---|
| | Pre-HTST | Filtration | from HTST / | Day 0 |
| Run | Mn (nM) | Mn (nM) | Filtration (%) | Mn (nM) |
| 1 | 93 | 76 | 18 | 56 |
| 2 | 93 | 67 | 31 | 42 |

3.2 Evaluation of Manganese Loss from HTST Heat Treatment and Filtration at Different pH Adjustment Targets

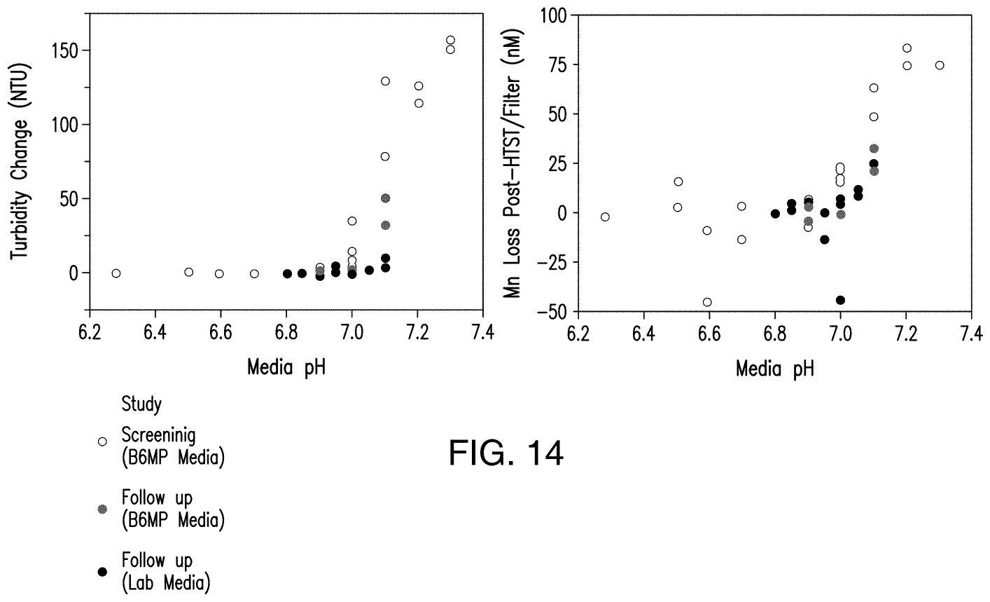

An initial screening study with the bench top Sand Bath HTST heat treatment method was used to evaluate changes in turbidity and manganese loss after heat treatment over a wide range of pH targets. Ocrelizumab production culture media was used for this study. The Sand Bath HTST heat treatment method represents a worst-case condition for HTST heat treatment due to the extended heat treatment time required to reach 102° C. compared to an HTST manufacturing skid. The change in turbidity before and after heat treatment and the manganese loss across heat treatment/filtration are shown in FIG. 14. Below a pH adjustment target of 7.00 for the production media prior to HTST, no significant increase in turbidity or decrease in Mn loss was observed. At a pH adjustment target of 7.00 or higher for the production media prior to HTST, a larger increase in turbidity and Mn loss was observed. This indicates that adjusting the media pH to a target below 7.00 prior to HTST heat treatment can be expected to decrease Mn loss across manufacturing HTST heat treatment and filtration operations.

A pilot scale HTST study was performed evaluating three pre-HTST pH adjustment targets of ˜6.30 (non-pH-adjusted media), 6.70, and 7.10. Ocrelizumab production culture media was used for this study. The Mn measurements and Mn loss across HTST and filtration are shown in Table 10. Consistent with the Sand Bath HTST heat treatment study, the ˜6.30 and 6.70 pre-HTST pH cases demonstrated a smaller Mn loss across HTST and filtration compared to the pH 7.10 case indicating that a lower pre-HTST pH target can help decrease the Mn loss observed across manufacturing HTST and filtration operations.

TABLE 10
Pilot Scale HTST Study Mn Measurements

| pH | | Post-HTST / | Mn | % |
|---|---|---|---|---|
| adjustment | Pre-HTST | filtration | loss | Mn |
| target | Mn (nM) | Mn (nM) | (nM) | loss |
| ~6.30 | 109 | 106 | 3 | 3.3 |
| 6.70 | 113 | 115 | −2 | −1.6 |
| 7.10 | 111 | 95 | 16 | 14.8 |

3.3 Impact of Pre-HTST pH Adjustment on 2 L Cell Culture Performance & Product Quality

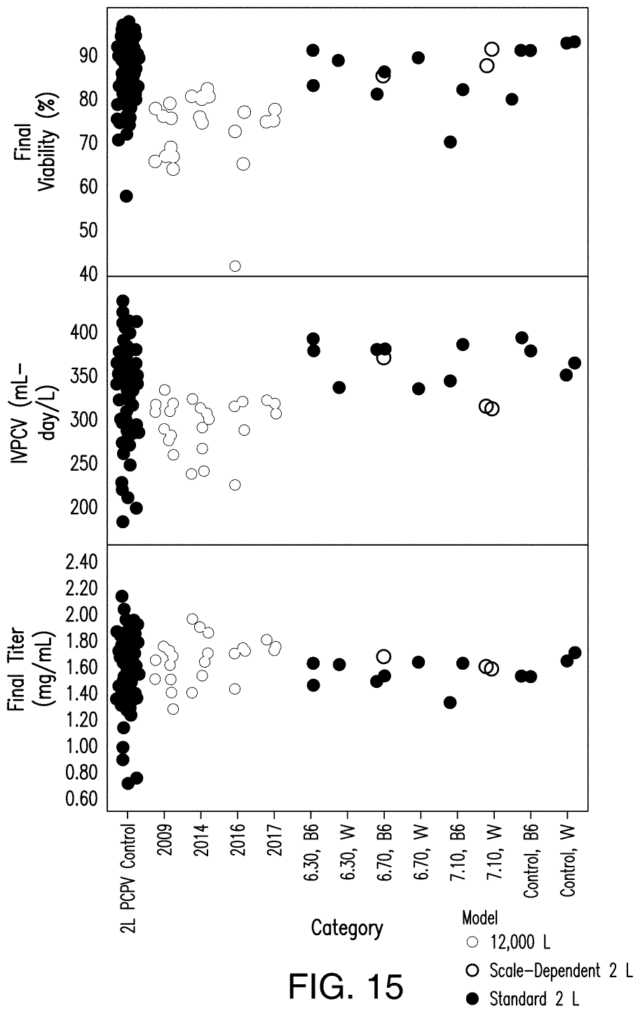

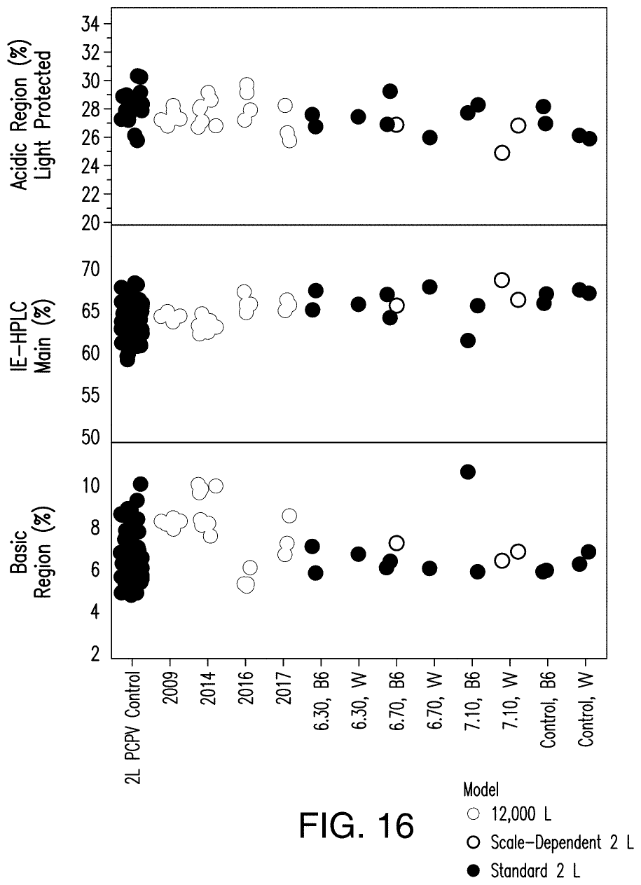

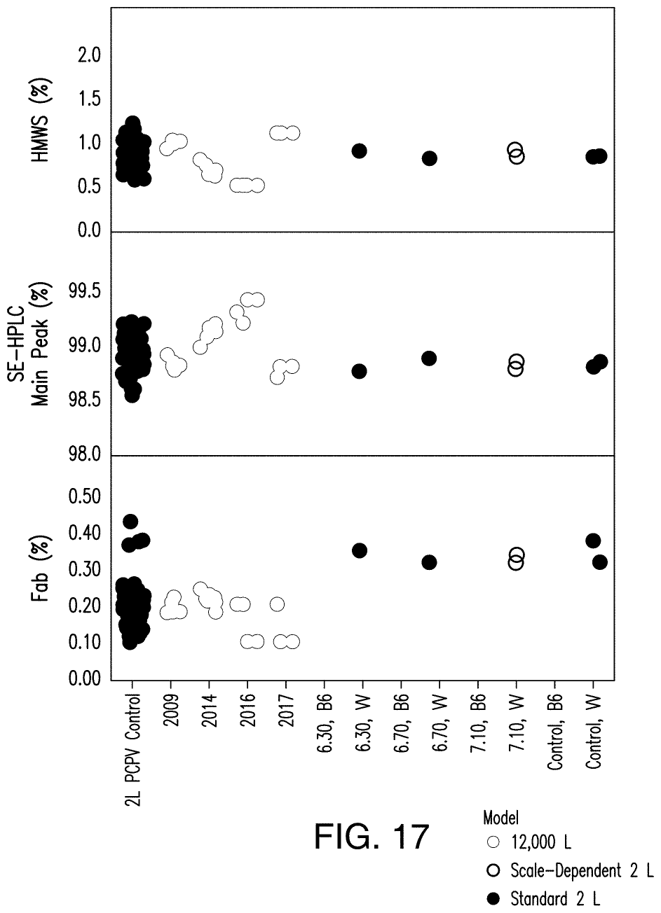

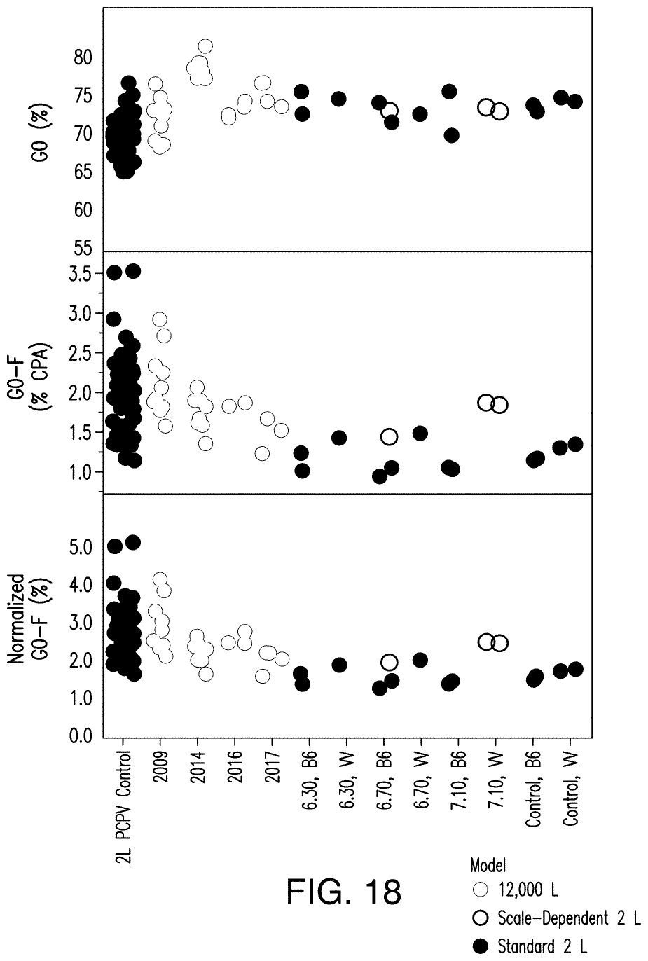

The production media treated in the pilot scale HTST study was used in two 2 L experiments. After HTST heat treatment and prior to filtration, the production media was adjusted to a final pH target of 7.10+/−0.10. This pH-adjustment step will occur after HTST and filtration and will target 7.15+/−0.10. Controls were included in each experiment using media prepared with the same Proteose Peptone 3 (PP3) and Genentech Essential Medium (GEM) Powder 2 raw material lots without HTST heat treatment. Select runs were executed with the scale-dependent 2 L model, which includes a 36-hour N−1 and N media hold and a modified sparging strategy to generate higher pCO₂ levels. The following results from these studies are compared to 2 L control runs performed during process characterization and validation (PC/PV) and historical manufacturing runs. 2 L control and manufacturing product quality data is from the affinity pool on respective AO assays. FIG. 15 shows KPIs. FIG. 16-FIG. 18 show product quality. KPIs, charge-related variants, size-related variants, and glycans showed no significant impact from varying the pre-HTST pH target between ˜6.30, 6.70, and 7.10. The studies indicate that cell culture performance and product quality will not be impacted by changing the pre-HTST pH target between 6.30 to 7.10 with a post-HTST pH adjustment to 7.10+/−0.10.

3.4 Effect of pH Adjustment on Osmolality in Ocrelizumab Production Media

The osmolality change from sodium carbonate addition for pH adjustment is shown in Table 11 and Table 12 for the sand bath HTST and initial pilot scale HTST studies. Both the osmolality prior to HTST heat treatment at a pH of 6.90 and final osmolality after final pH adjustment to 7.10 was observed to be within the current production media target osmolality.

TABLE 11
pH, Osmolality, and Sodium Carbonate Addition for
Ocrelizumab Production Media pH Adjustment to pH 6.90.

| | | | Pre- | Post- | | 1M |
|---|---|---|---|---|---|---|
| | Pre- | Post- | Adjust | Adjust | Osmo | Na₂CO₃ |
| | Adjust | Adjust | Osmo | Osmo | Change | Used |
| Run | pH | pH | (mOsm/kg) | (mOsm/kg) | (mOsm/kg) | (mL/L) |
| 1 | 6.35 | 6.89 | 317 | 329 | 12 | 4.3 |
| 2 | 6.32 | 6.90 | 325 | 340 | 15 | 5.3 |
| 3 | 6.30 | 6.90 | 321 | 324 | 3 | 3.1 |
| 4 | 6.27 | 6.90 | 320 | 324 | 4 | 2.9 |
| 5 | 6.30 | 6.90 | 321 | 328 | 7 | 4.7 |
| Average | 6.31 | 6.90 | 321 | 329 | 8 | 4.1 |

TABLE 12
pH, Osmolality, and Sodium Carbonate Addition for Ocrelizumab
Production Media pH Adjustment from pH 6.90 to pH 7.10.

| | | | Pre- | Post- | | 1M |
|---|---|---|---|---|---|---|
| | Pre- | Post- | Adjust | Adjust | Osmo | Na₂CO₃ |
| | Adjust | Adjust | Osmo | Osmo | Change | Used |
| Run | pH | pH | (mOsm/kg) | (mOsm/kg) | (mOsm/kg) | (mL/L) |
| 1 | 6.92 | 7.09 | 329 | 329 | 0 | 0.9 |
| 2 | 6.88 | 7.10 | 340 | 345 | 5 | 1.9 |
| Average | 6.90 | 7.10 | 334 | 337 | 3 | 1.4 |

3.5 Recommendations for Ocrelizumab Production Media Pre-HTST pH and Osmolality Targets and Target Ranges

Based on the results summarized in this Example, the recommended ocrelizumab production media pre-HTST pH adjustment target is 6.90+/−0.10 or 6.70+/−0.10 with an osmolality alert range of 320-350 mOsm/kg. The small-scale study results show no impact to cell culture performance or product quality and support the use of a pre-HTST pH in the range of 6.30-7.10. In addition, historical manufacturing runs support a pre-HTST pH target of 7.15+/−0.10; therefore, the overall acceptable pre-HTST pH target range is 6.30-7.25. After completion of HTST heat treatment, filtration, and batching of the 12,000 L bioreactor, the ocrelizumab production media will require a pH adjustment to the final target pH of 7.15+/−0.10. Upon final pH adjustment within the 12,000 L bioreactor, an osmolality check should be made to confirm the media is within the target osmolality of 340+/−20 mOsm/kg prior to inoculation of the production culture.

3.6 Use of Lower pre-HTST pH Set Point for Additional mAb

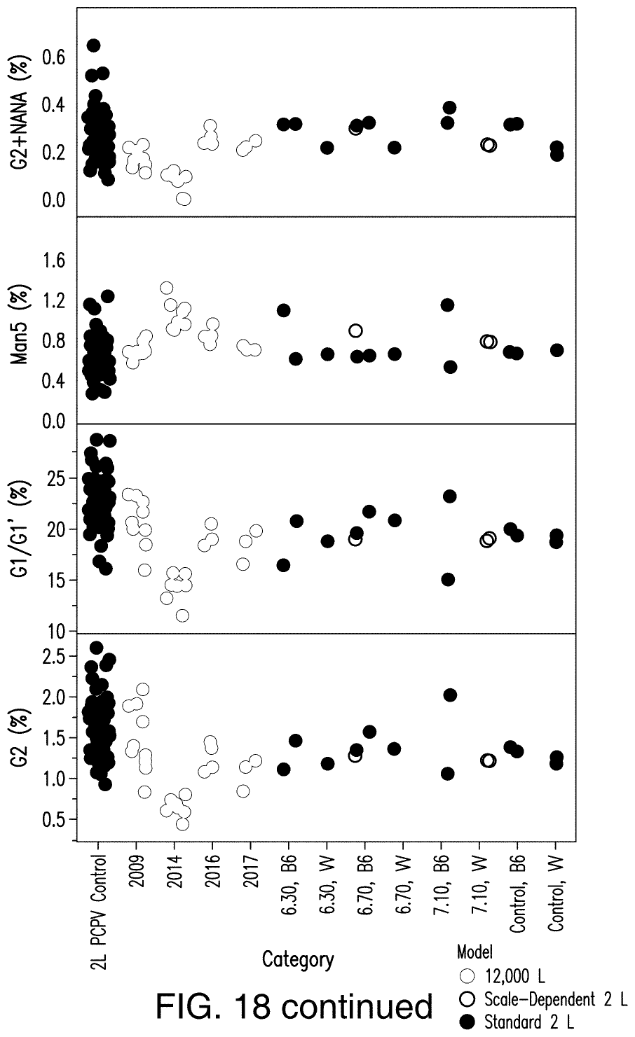

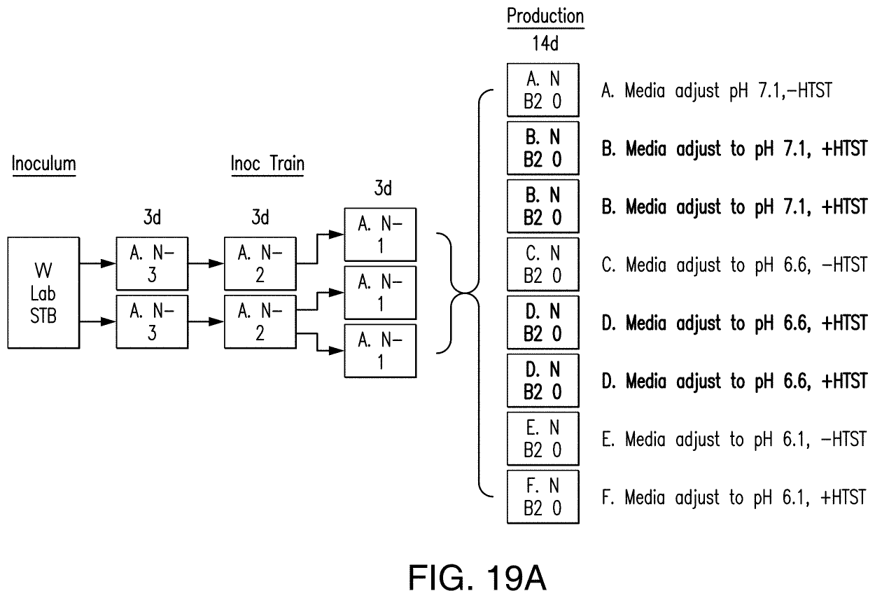

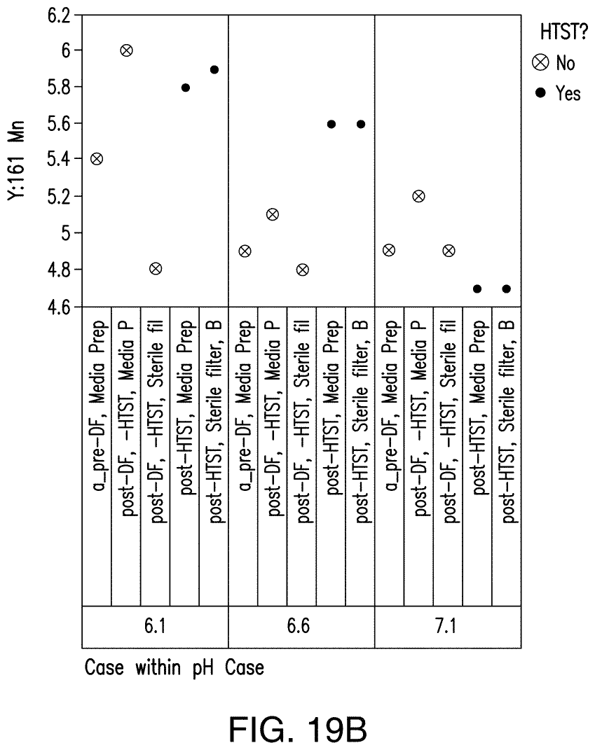

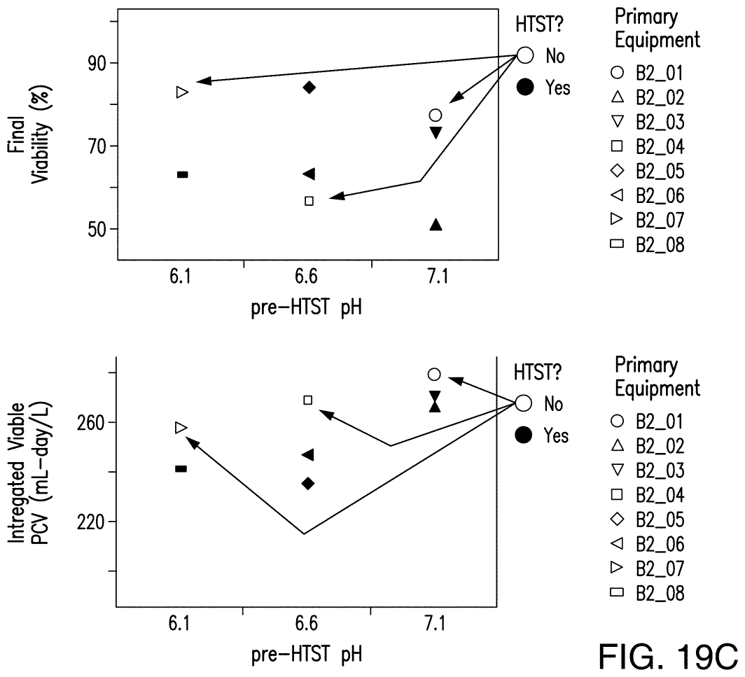

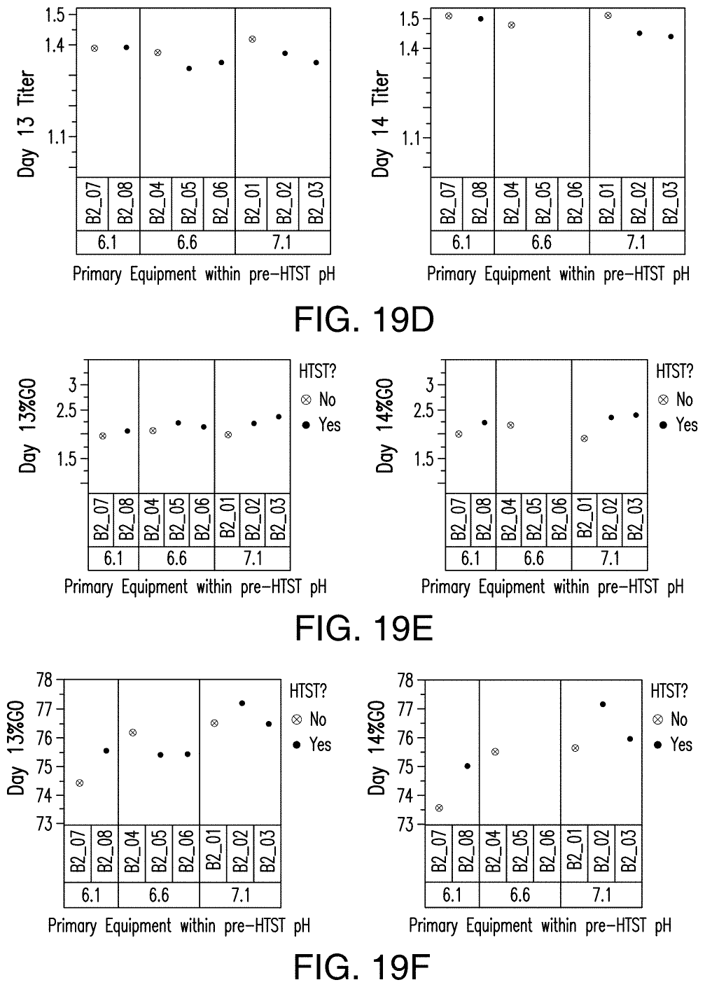

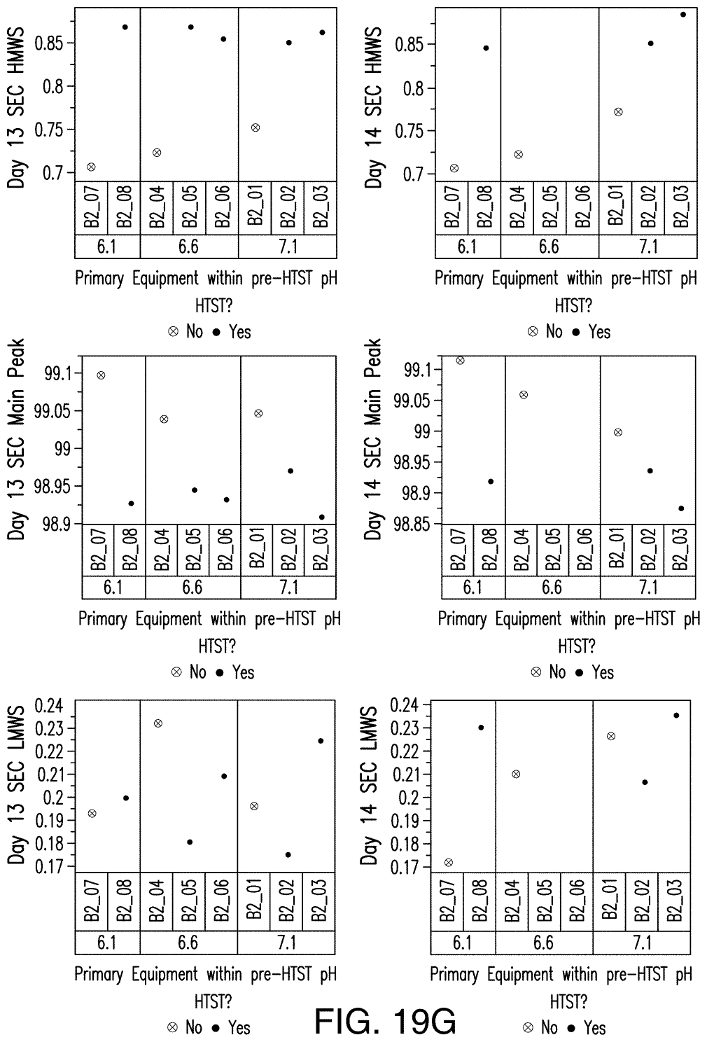

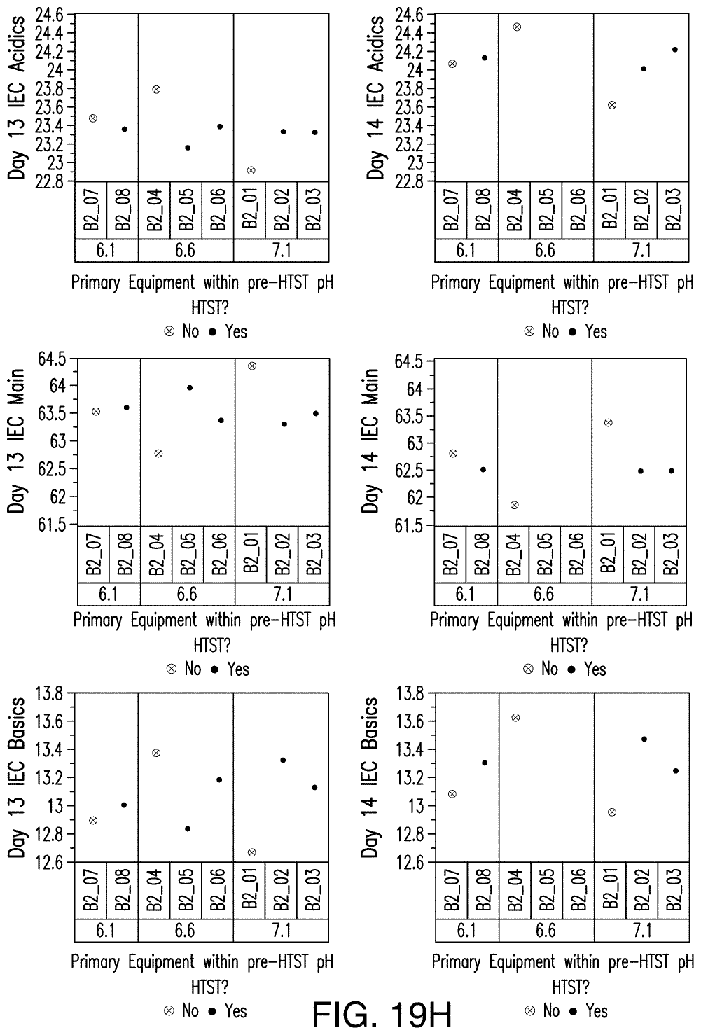

During manufacturing a preparation of production basal media for the additional mAb example, Antibody III, experienced the atypical HTST performance as observed in ocrelizumab (FIG. 13). In order to mitigate against future HTST performance issues as well as prevent loss of manganese due to atypical HTST performance, the media pH prior to HTST heat treatment and filtration was evaluated for the additional mAb. Media was prepared to the pH targets of 7.1, 6.6, and 6.1. Each media preparation was split for use with and without HTST heat treatment. FIG. 19B shows manganese results from the media preparations. The blue symbols show the manganese levels in the HTST heat treated media. There is a clear trend with increasing media pH target and decreased manganese after HTST heat treatment. This indicates that at higher media pHs, higher manganese loss is observed across HTST heat treatment and filtration. This result is consistent with the studies performed for ocrelizumab.

A 2 L study was performed to evaluate the impact of a lower media pH prior to HTST heat treatment and filtration on cell culture performance and product quality. KPI results are shown in FIG. 19C and FIG. 19D. Product quality results are shown in FIG. 19E through FIG. 19H. The study indicates that cell culture performance and product quality for the additional mAb will not be impacted by changing the pre-HTST pH target between 6.10 and 7.10.

Based on results of this study, the pre-HTST heat treatment and filtration media pH target for the additional mAb was decreased to 6.6.

### Example 4: pCO₂, Manganese, Media Hold, Osmolality, and Na+ to Modulate Fucosylation

4.1 Introduction

Amongst cell culture process parameters with unknown impact on glycosylation (e.g., galactosylation and/or afucosylation), partial pressure of carbon dioxide (pCO₂) in the culture fluid is of substantial interest because pCO₂ levels can vary across bioreactor scales; therefore, maintaining a comparable pCO₂ profile is a frequently-encountered challenge during process scale-up. Previous studies have shown that pCO₂ levels can affect cell growth, productivity, and recombinant protein glycosylation in mammalian cell cultures (Darja et al., (2016), Journal of Biotechnology, 219, 98-109; deZengotita et al., (1998), Cytotechnology, 28, 219-227; Gray et al., (1996), Cytotechnology, 22 (1-3), 65-78; Kimura et al., (1996), Biotechnol and Bioeng, 62, 152-160; Kimura et al., (1997), Biotechnol Prog, 13, 311-317; Schmelzer et al., (2002), Biotechnol Prog, 18, 346-353; Zhu et al., (2005), Biotechnol Prog, 21 (1), 70-77). Although these studies did not demonstrate an impact of pCO₂ on afucosylation, the pCO₂ profiles evaluated are not representative of those encountered in large-scale bioreactor cultures.

To better understand and establish robust control of afucosylation across bioreactor scales, the effect of pCO₂ and its potential interaction with other process levers on the afucosylation of a mAb produced by a recombinant Chinese Hamster Ovary (CHO) cell line was investigated. The following approach was applied: (1) construct a small-scale (3-L) bioreactor model capable of maintaining different levels of pCO₂ while keeping other process parameters constant; (2) examine the effect of pCO₂ and its interactions with other process parameters, e.g., manganese and media hold, on mAb afucosylation; and (3) investigate the potential underlying mechanism(s) behind any observed effect of pCO₂ and other process levers on mAb afucosylation. Mn was selected because it is a cofactor for multiple glycosylation enzymes (Rouiller et al., (2014), Biotechnol Prog, 30 (3), 571-583) and has been used to modulate glycosylation levels in CHO cell culture studies (Gramer et al., (2011), Biotechnol Bioeng, 108 (7), 1591-1602; Surve et al., (2014), Biotechnol Prog., 31 (2): 460-647), even though it has not previously shown any impact on afucosylation and is not known to be a cofactor for a1,6-fucosyltransferase (FUT8). Media hold was studied because production media is held in bioreactors prior to inoculation in large-scale operations and its impact on product quality attributes has not been previously reported.

4.2 Materials and Methods

4.2.1 Cell Culture

The same recombinant CHO cell line expressing a mAb of the immunoglobulin G1 (IgG1) subclass was used in all studies reported herein. Cells were thawed and expanded to inoculate production cultures in 3-L glass bioreactors (Applikon) as previously described (Yuk et al., (2015), Biotechnol Prog, 31 (1), 226-237). The set points for temperature, pH, and dissolved oxygen (DO) in the bioreactors were controlled by Finesse SmartController with TruBio DeltaV (Thermo Fisher Scientific). Temperature, pH, and DO for all production cultures were maintained at 37° C., 7.15, and 30% (of air saturation) on the first day; at 34° C., 7.15, and 30% between days 1 and 3; and at 34° C., 7.00, and 30% DO from day 3 thereafter. Three days post-inoculation, a concentrated nutrient feed was added to the production cultures at a 1:7 (v/v).

The following parameters were studied: Mn supplementation, L-fucose supplementation, media hold, pCO₂ level, and osmolality level (using sorbitol and NaCl as osmolality titrants). The basal medium used for inoculum train and production cultures contained a nominal amount of Mn and no L-fucose. Supplemental Mn and/or L-fucose were added immediately after inoculating the production cultures to achieve the target day 0 concentration as described for the study. Media hold was executed by maintaining basal medium (used for N−1 inoculum train and production cultures) for 36 hours in a separate 3-L bioreactor at 37° C. with air sparge (10 sccm), agitation (75 rpm), and one-sided (CO₂ only) pH control. Osmolality was adjusted by adding stock solutions of NaCl (100 g/L) or sorbitol (182 g/L).

4.2.2 High and Low pCO₂ Model Configurations

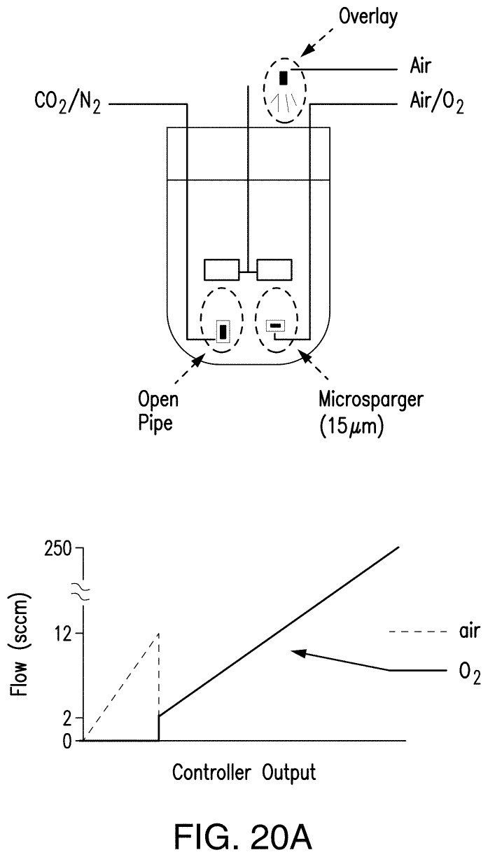

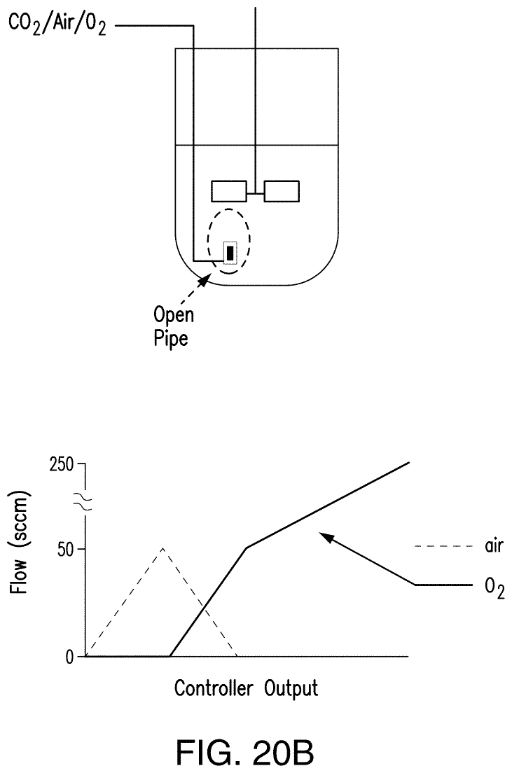

To achieve different pCO₂ profiles, bioreactor configurations were modified to generate a high pCO₂ model (FIG. 20A) and low pCO₂ model (FIG. 20B). In both models, pH was controlled by sparging CO₂ through an open pipe (5 mm inner diameter) to decrease pH, and by adding Na₂CO₃ to increase pH as previously described (Hsu et al., (2012), Cytotechnology 64 (6):667-678).

For the high pCO₂ model (FIG. 20A), the bioreactor working volume was >1.9 L. DO was controlled by supplying air/O₂ through a microsparger (15 μm pore size). The DO controller setup used air to control DO with a minimum 2 sccm output; after the air output reached 12 sccm, DO control was switched to 02 with a minimum 2 sccm output.

For the low pCO₂ model (FIG. 20B), the bioreactor working volume was <1.5 L. DO was controlled by sparging air/O₂ through the same open pipe used for CO₂. The DO controller setup used air output at a minimum sparge of 10 sccm and a maximum sparge of 50 sccm. After the air output reached 50 sccm, 02 sparge increased and air sparge decreased. When 02 sparge reached 50 sccm, air was turned off and the 02 output increased, as required, up to a maximum of 250 sccm.

4.2.3 Cell Culture Analysis

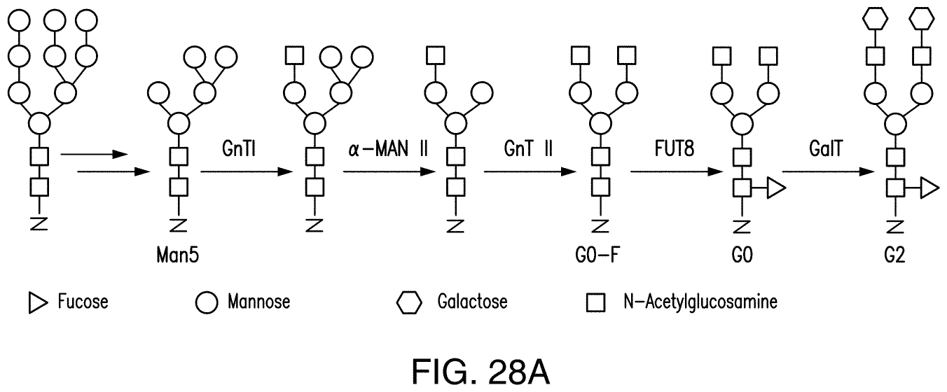

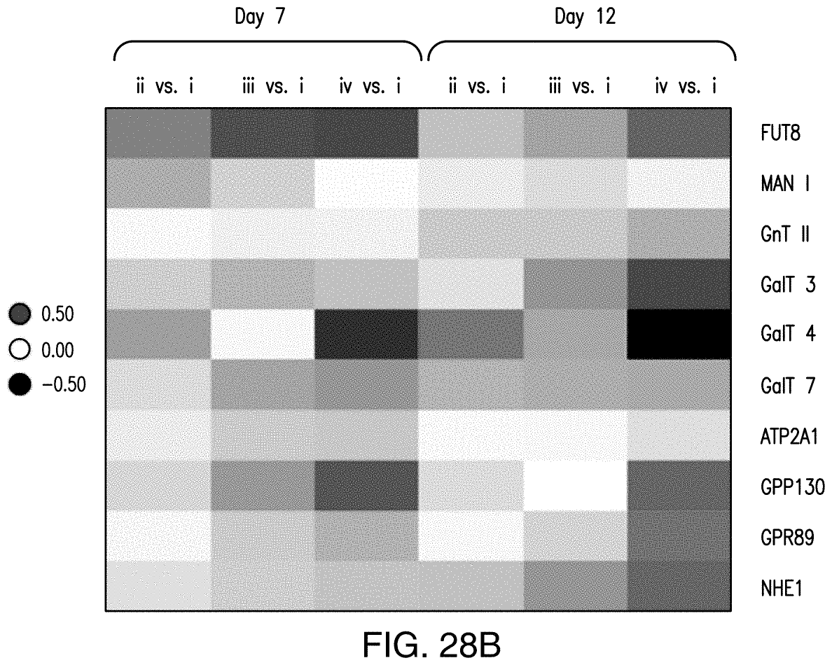

Packed cell volume (PCV), viable cell concentration, culture viability, pH, DO, pCO₂, glucose, lactate, osmolality, Na+, ammonium, mAb product titer, glycosylation variants, charge variants, size variants, and Mn concentration (by inductively-coupled plasma mass spectrometry) were measured as previously described (Hsu et al., (2012), Cytotechnology 64 (6):667-678; Yuk et al., (2015), Biotechnol Prog, 31 (1), 226-237). Structures of the N-linked glycosylated species are detailed in Thomann et al. (2016) and illustrated in FIG. 28A. Afucosylation was normalized to G0F and defined as:

$$Afucosylation ( \% ) = \frac{G 0}{G 0 F + G 0} \times 100 \%$$

4.2.4 Intracellular pH Analysis

Intracellular pH (pHi) was measured using SNARF-4F 5-(and-6)-carboxylic acid, acetoxymethyl ester acetate (SNARF-4F) (Molecular Probes; Cat #S23921, Thermo Fisher Scientific). SNARF-4F is the fluorinated derivative of carboxy SNARF-1 and has a pKa value of ˜6.4. The pHi measurement and calculation methods were based on Reynolds et al (Reynolds et al., (1996), Cytometry, 25, 349-357) and deZengotita et al. (deZengotita et al., (2002), Biotechnol Bioeng, 77 (44), 369-380).

To prepare for pHi measurement, fresh media was pre-equilibrated to the desired condition for a minimum of 6 hours. For the pCO₂ titration cases, media was equilibrated in the TAP ambr 15 system (Sartorius Stedim Biotech) controlled at 37° C. and 600 rpm agitation with CO₂ sparging to reach the desired pCO₂ levels. Next, pH was adjusted to 7.0 using 0.5 M Na₂CO₃ and osmolality was adjusted to −400 mOsm/kg using 100 g/L NaCl. For the osmolality titration cases, media osmolality was adjusted using 100 g/L NaCl and placed in an incubator controlled at 37° C., 5% CO₂, and 50 rpm agitation (2.5 cm orbit) overnight. Cells were pelleted (0.5 million cells, 200 g, 2 min) and washed twice in phosphate buffered saline (PBS). The pellets were quickly re-suspended in fresh media pre-equilibrated to the desired condition and SNARF-4F was added (1.5 μg/mL final concentration). The cell-dye mixture was incubated (30 min) under the same conditions as the pre-equilibrated media. pHi was measured immediately using the Attune NxT Flow Cytometer (Thermo Fisher Scientific) with a 488 nm laser and detection at 585 nm and 640 nm. Extracellular pH, pCO₂, osmolality, and Na⁺ were measured using the Nova Bioprofile FLEX. A pH calibration curve was generated using cells dyed in known pH buffers in the presence of Nigericin (Sigma Cat #N7143, Sigma-Aldrich) as previously described (Salvi et al., (2002), AAPS PharmSci, 4 (4), 1-8).

4.2.5 Proteomic Analysis

For proteomic analysis by mass spectrometry (MS), cells from production cultures on days 7 and 12 were pelleted (10 million cells, 200 g, 2 min), washed twice with PBS, flash-frozen on dry ice, and stored at −80° C. until analysis. Proteins were extracted from each sample, digested to peptides, labeled with Tandem Mass Tags (TMTs) as previously described (Vildhede et al., (2018), Drug Metab Dispos, 46 (5), 692-696), and analyzed using an Orbitrap Lumos mass spectrometer (Thermo Scientific) with an SPS-MS3 method (McAlister et al., (2014), Anal Chem, 84 (16), 7150-7158).

Assignment of MS/MS spectra was performed using the MASCOT search algorithm to search against all entries for Cricetulus griseus (Chinese hamster) in UniProt (downloaded June 2016). A search of all tryptic peptides (2 missed cleavages) was performed and a precursor tolerance of 50 ppm was used to limit the number of candidate peptides, while a 0.8 Da tolerance was used to match MS/MS data collected in the ion trap. Static modifications included TMT on the N-terminus of peptides and lysine residues (+229.16293) and cysteine alkylation (57.0215), while variable modifications included methionine oxidation (15.9949) and TMT labeling of tyrosine (229.1629). Peptide spectral matches were filtered to a 2% false discovery rate using a target decoy approach scored with a linear discrimination analysis algorithm before filtering to a 2% false discovery rate at the protein level as previously described (Kirkpatrick et al., (2013), PNAS, 110 (48), 19462-19431).

Quantitative values were extracted and corrected for isotopic impurities using Mojave (Zhuang et al., (2013), Sci Signal, 6 (271), 1-11). Additionally, quantitative events with a precursor purity <0.7 (±0.25 Da) or sum intensity <50,000 were discarded before quantitative values were normalized and converted to “relative abundance” values using custom scripts in R. Relative abundance values were calculated for each protein by dividing the sample intensity by the total intensity for the protein and then normalizing the result to 100. Following data normalization, principal component analysis (PCA) was performed using a custom script in R. To determine pathway enrichment within the dataset, UniProt identifiers were converted to homologous Mouse, Rat, or Human identifiers and processed by Ingenuity Pathway Analysis (IPA; QIAGEN Inc.) as previously described (Kramer et al., (2014), Bioinformatics, 30 (4), 523-530).

4.3 Results and Discussion

4.3.1 Low and High pCO₂

To develop a small-scale bioreactor model capable of maintaining different pCO₂ levels, the CO₂ stripping rate in 3-L bioreactors was regulated (FIG. 20). Although NaHCO₃ concentration in media can be adjusted to alter pCO₂ levels in bioreactor cultures (Goudar et al., (2006), Biotechnol Bioeng, 96 (6), 1107-1117; Zhu et al., (2005), Biotechnol Prog, 21 (1), 70-77), the bioreactor gas sparge rate was regulated instead because it is an effective way to modulate CO₂ stripping (and hence pCO₂ levels) while keeping agitation and vessel aspect ratio constant.

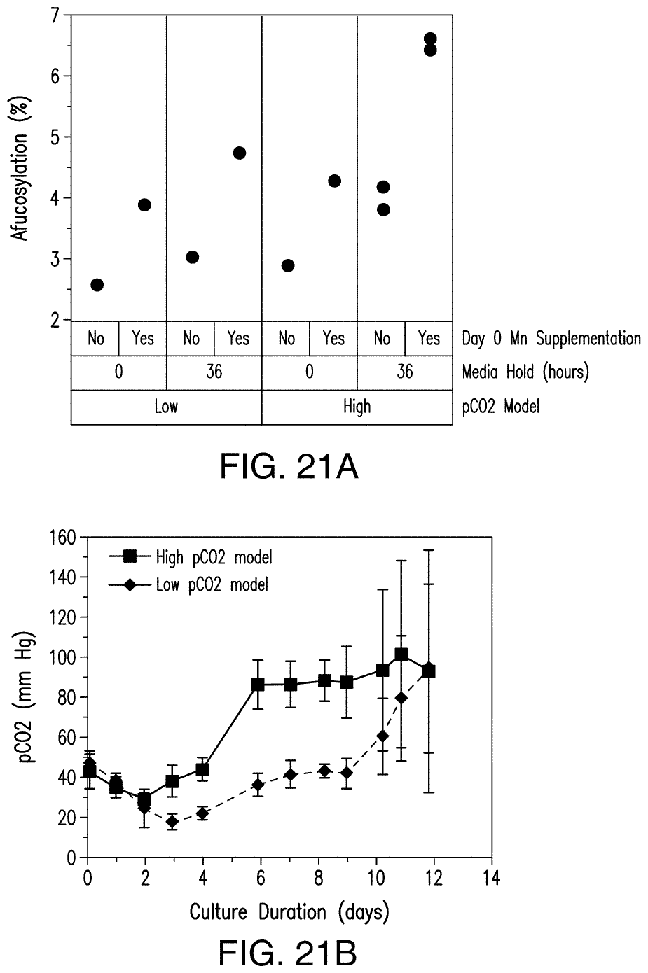

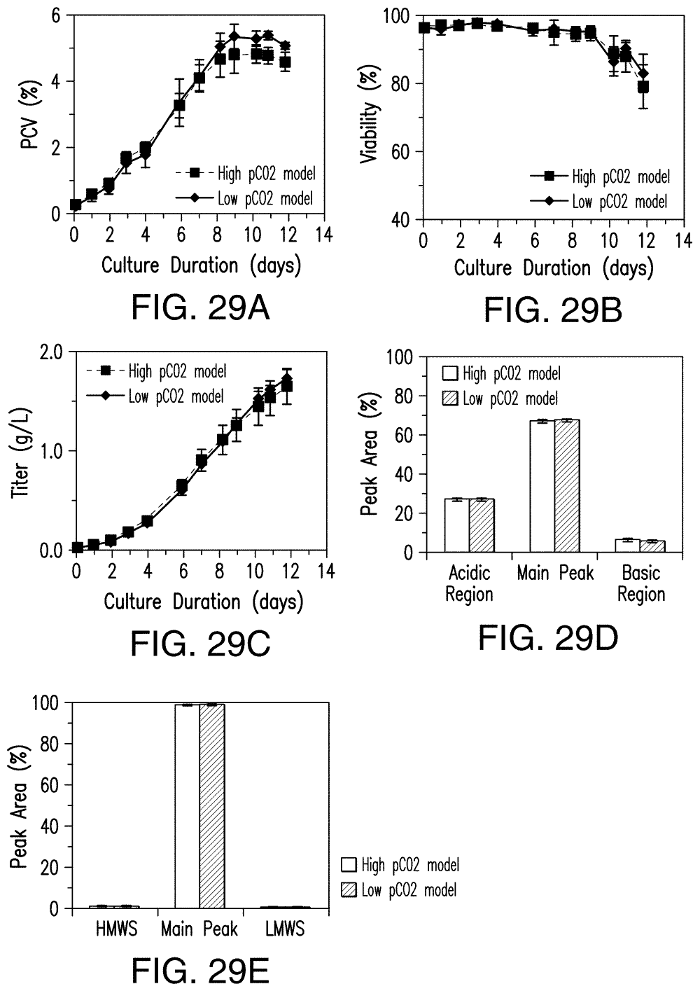

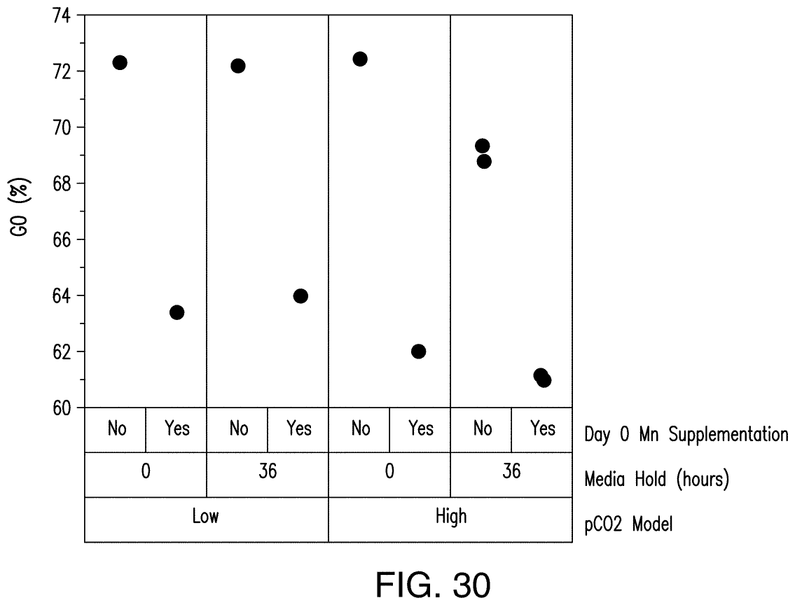

To achieve a low gas sparge rate while maintaining a sufficient k_{L}a to support the cellular metabolic demand for oxygen, fritted microsparger for DO control in the high pCO₂ model was used. The microsparger results in a small gas bubble size thus increasing total gas-liquid surface area interface. To increase the residence time of CO₂ and further decrease CO₂ stripping (Matsunaga et al., (2009), Journal of Bioscience and Bioengineering, 107 (4), 419-424), the high pCO₂ model was operated at a higher working volume (≥1.9 L). The low pCO₂ model utilized an open pipe sparger and lower working volume (≤1.5 L) to increase CO₂ stripping. These differences in bioreactor configurations resulted in the desired separation in pCO₂ levels between the two models (FIG. 21B). Even though high pCO₂ has been shown to impact hybridoma cell growth, viability, and antibody production (deZengotita et al., (1998), Cytotechnology, 28, 219-227), no negative effect on these attributes and product quality was observed for the CHO cell line used in this study (FIG. 29). This is believed to be the first report to demonstrate regulation of pCO₂ levels for CHO cultures in the same small-scale bioreactors without manipulating the NaHCO₃ concentration in the culture medium.

4.3.2 Effects of pCO₂ Level, Media Hold, and Supplemental Mn on Afucosylation

Using the high and low pCO₂ bioreactor models, the effect of and potential interactions between pCO₂ level, media hold, and supplemental Mn on afucosylation in a full factorial design of experiment (DOE) was examined. Consistent with the established impact of Mn on galactosylation (Gramer et al., (2011), Biotechnol Bioeng, 108 (7), 1591-1602), the agalactosylated species (G0F) decreased in the background of supplemental Mn (FIG. 21). Afucosylation was ˜1% higher with Mn supplementation in the low pCO₂ background (Figure This result is unexpected because Mn is not known to impact fucosylation. For example, while several other divalent cations significantly inhibited FUT8 activity, Mn did not (Kaminska et al., (1998), Glycoconjuagte Journal, 15, 783-788). Afucosylation was ˜2% higher with media hold or high pCO₂ in the presence of supplemental Mn (FIG. 21A). Afucosylation was highest (by ˜4%) in the presence of supplemental Mn, media hold, and high pCO₂, indicating an interaction between these three factors.

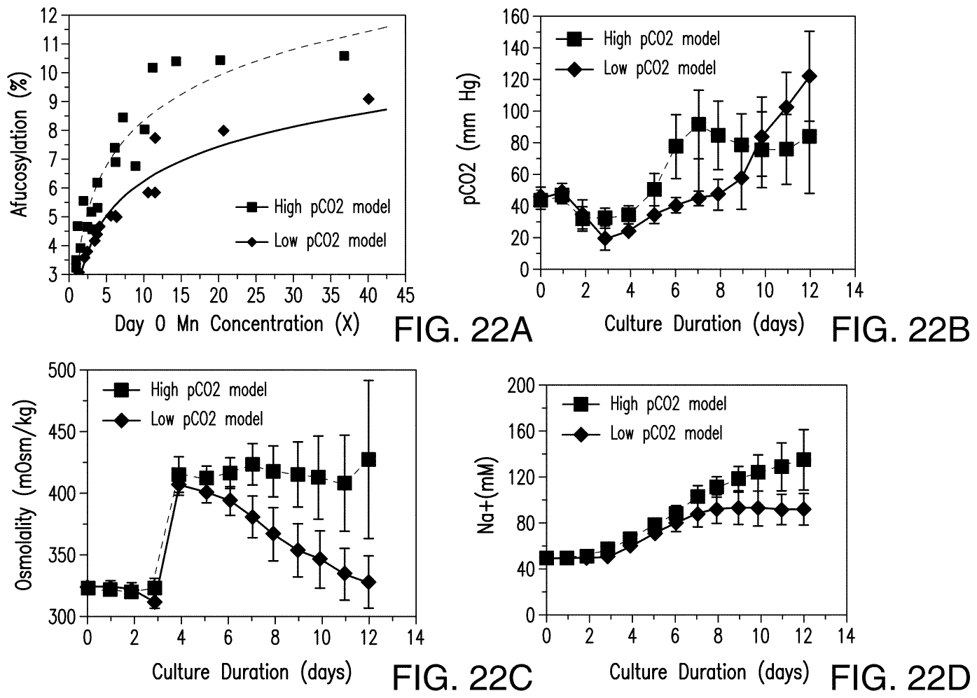

This observed interaction was confirmed at multiple Mn supplementation levels using both high and low pCO₂ models (FIG. 22). The increase in afucosylation was more pronounced in the high pCO₂ model with media hold at all Mn levels (FIG. 22A). This example demonstrates the effect of and interaction between Mn, media hold, and pCO₂ level in modulating afucosylation. Therefore, the Mn, media hold, and pCO₂ level can each modulate afucosylation on their own, but their impact on afucosylation is magnified when they work in combination.

4.3.3 De-confounding the Effects of High pCO₂, Osmolality, and Na⁺ on Afucosylation

Higher culture osmolality and Na⁺ was observed in the high pCO₂ model compared with the low pCO₂ model (FIG. 22C-22D). This was can be explained by the fact that CO₂ in solution equilibrates to form H⁺ and HCO₃⁻ as shown by the simplified equilibrium equation (Equation 1):

CO₂+H₂O↔H⁺HCO₃⁻ Equation 1

In a pH-controlled environment, base (Na₂CO₃ in this study) is added to the bioreactor to neutralize H+, thereby driving the equilibrium to the right of Equation 1 and increasing culture osmolality and HCO₃⁻ concentration (deZengotita et al., (2002), Biotechnol Bioeng, 77 (44), 369-380). Na⁺ levels are also increased through Na₂CO₃ addition. These observations beg the question whether the increase in mAb afucosylation was caused by high pCO₂, osmolality, or Na⁺.

Attempts to de-confound the effect of high pCO₂ from that of osmolality on afucosylation have led to conflicting results in the literature. The observations have ranged from minimal impact on afucosylation from increasing pCO₂ and/or osmolality (Kimura et al., (1997), Biotechnol Prog, 13, 311-317; Schmelzer et al., (2002), Biotechnol Prog, 18, 346-353) to decrease in afucosylation with increasing osmolality (Konno et al., (2012), Cytotechnology, 64, 249-265). In these previous studies, the production cultures were initiated at high pCO₂ and/or osmolality; this is not representative of the typical conditions in bioreactors, in which pCO₂ and osmolality are initially low and subsequently increase over the course of the production culture (Hsu et al., (2012), Cytotechnology 64 (6):667-678).

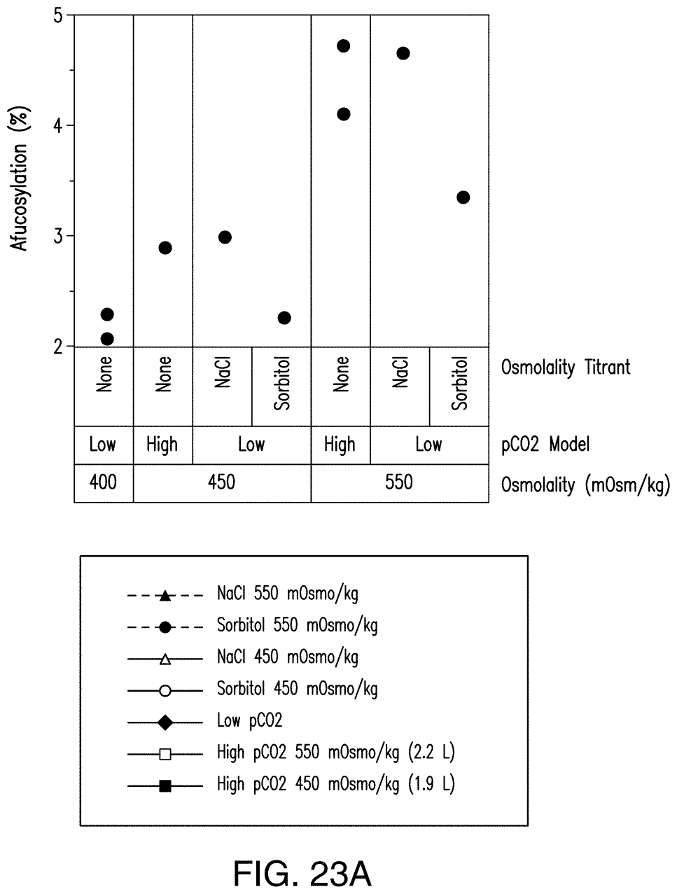

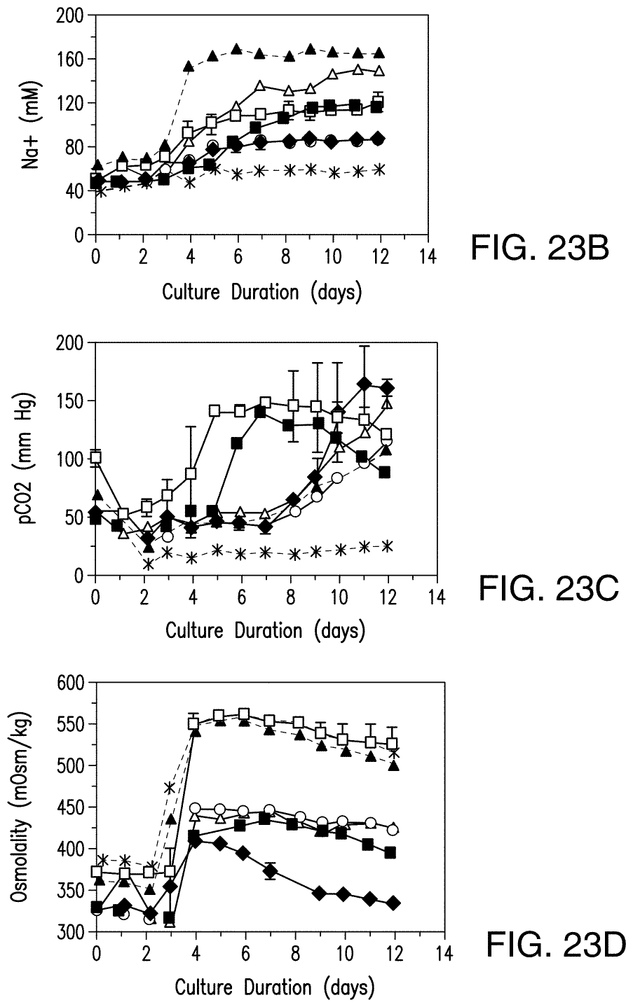

To discern the effect of pCO₂ from that of osmolality and Na⁺ in an environment that is more representative of typical CHO bioreactor cultures, osmolality in the low pCO₂ model was titrated with either NaCl or sorbitol to match the time-course osmolality profile of the production culture in the high pCO₂ model operated at 1.9 L (peak osmolality ˜450 mOsm/kg) and 2.2 L (peak osmolality ˜550 mOsm/kg) (FIG. 23). Afucosylation increased with increasing osmolality for all cases. At both target peak osmolality levels, afucosylation was similar between the NaCl titration (low pCO₂) case and the high pCO₂ case, whereas afucosylation was ˜1% lower for the sorbitol titration (low pCO₂) case. These results indicate that the afucosylation increase observed in the high pCO₂ model is likely due to the higher concentration of Na⁺ from the addition of Na₂CO₃ used for pH control rather than high pCO₂ or osmolality alone.

4.3.4 Intracellular pH Changes: High pCO₂ and High Osmolality/Na⁺

To investigate the mechanism behind the larger afucosylation increase in the background of high pCO₂ and/or Na⁺, pHᵢ was measured for the recombinant CHO cell line when subjected to different pCO₂ and osmolality/Na⁺ levels. CO₂ can diffuse across the cell membrane (Endeward et al., (2014), Frontiers in Physiology, 4, 1-21), encounter the equilibrium described in Equation 1 inside the cell, and thus lower pHi. In addition, Na⁺ from base addition (Na₂CO₃) to control extracellular pH can affect pHᵢ through Na⁺/H⁺ exchange (Orlowski et al., (1997), J Biol Chem, 272(36), 22373-22376) and/or Na⁺-dependent Cl⁻/HCO₃⁻ exchange (Reusch et al., (1995), American Physiological Society, C147-C153). A change in pHᵢ can impact enzyme expression (Bumke et al., (2003), Proteomics, 3(5), 675-688). Moreover, each enzyme has a pH range for optimal activity. Therefore, a shift in pHᵢ (due to high pCO₂ and/or Na⁺) can impact the expression and/or activity of enzymes involved in fucosylation.

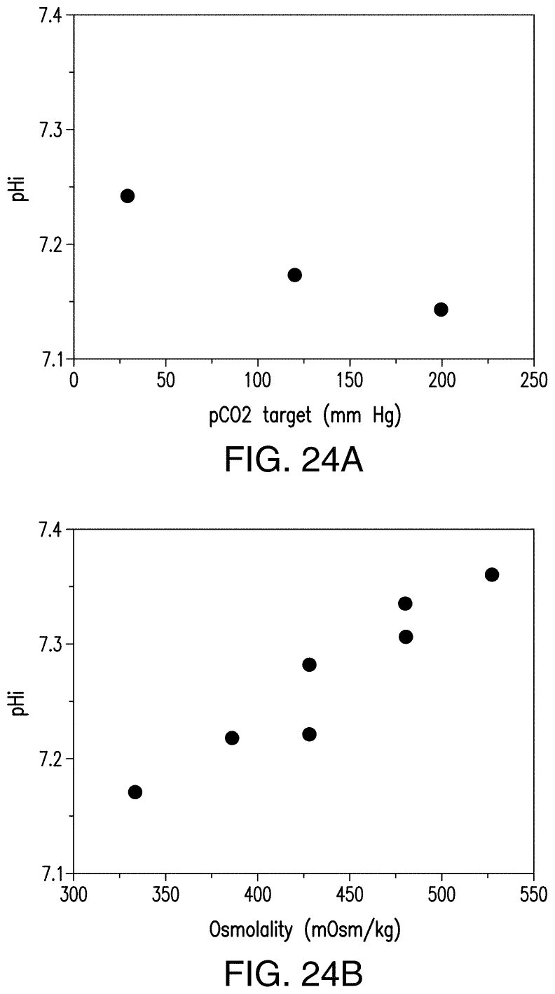

To test this hypothesis, pHᵢ was measured for the recombinant CHO cell line at different levels of pCO₂ and osmolality (using NaCl to titrate osmolality). pHᵢ decreased with increasing pCO₂ level (FIG. 24A) and increased with increasing osmolality/Na⁺ (FIG. 24B). These data show that both pCO₂ and osmolality/Na⁺ can affect pHᵢ, consistent with previous findings (deZengotita et al., (2002), Biotechnol Bioeng, 77 (44), 369-380; Reusch et al., (1995), American Physiological Society, C147-C153).

4.3.5 Global Proteomic Analysis

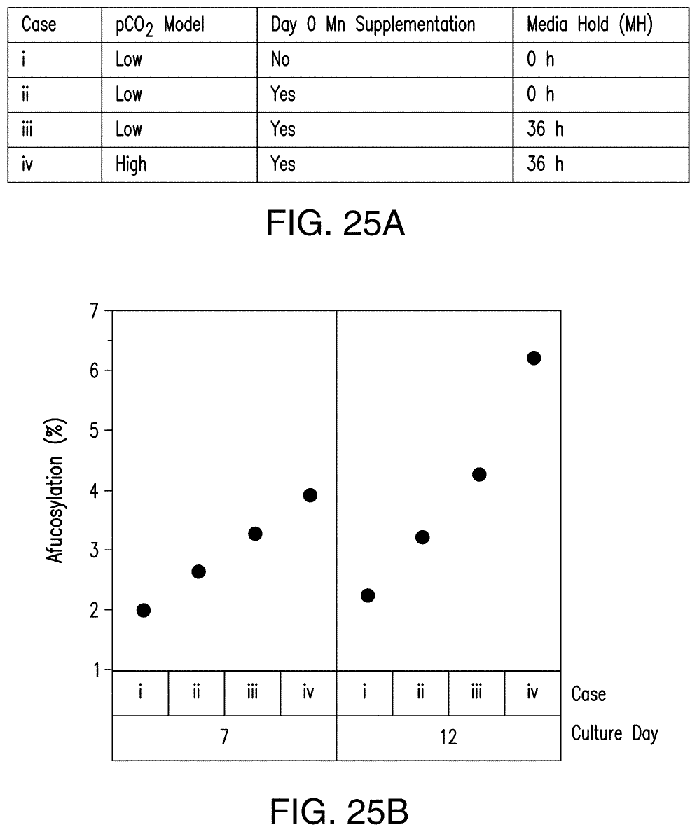

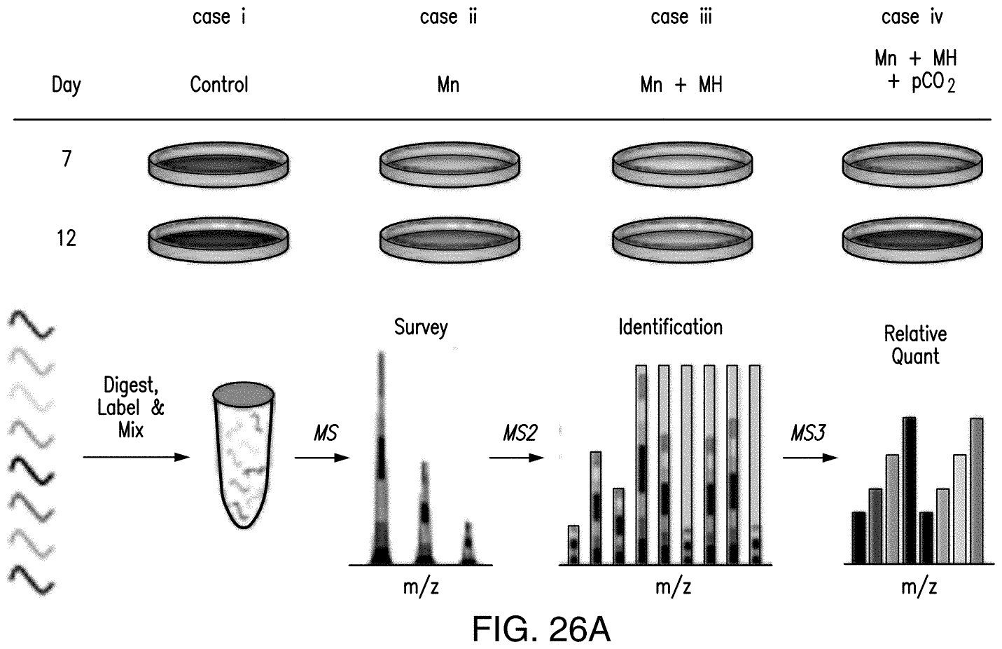

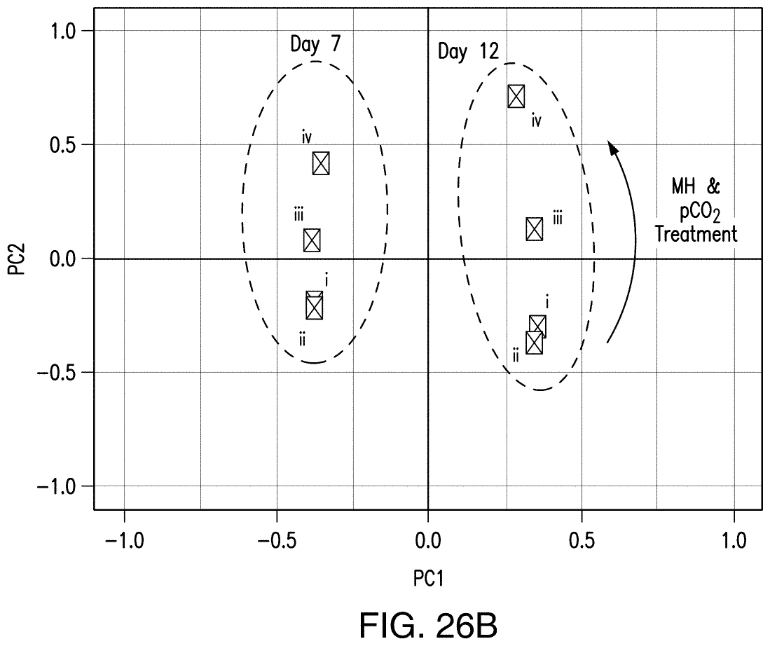

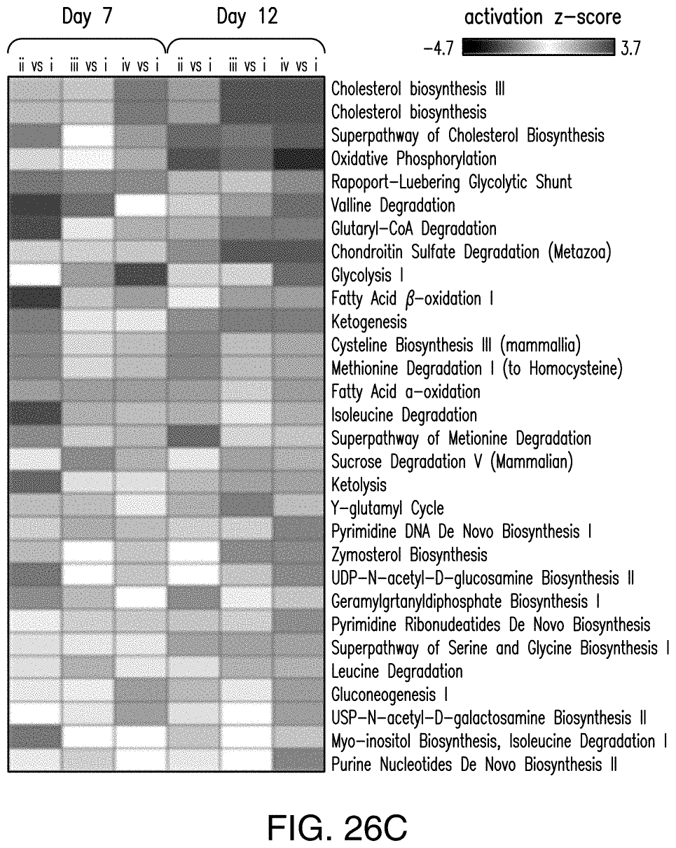

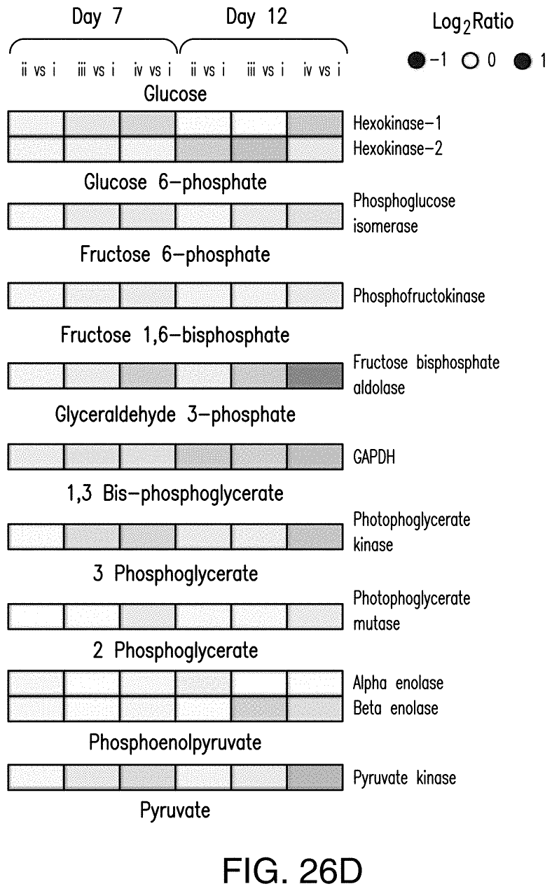

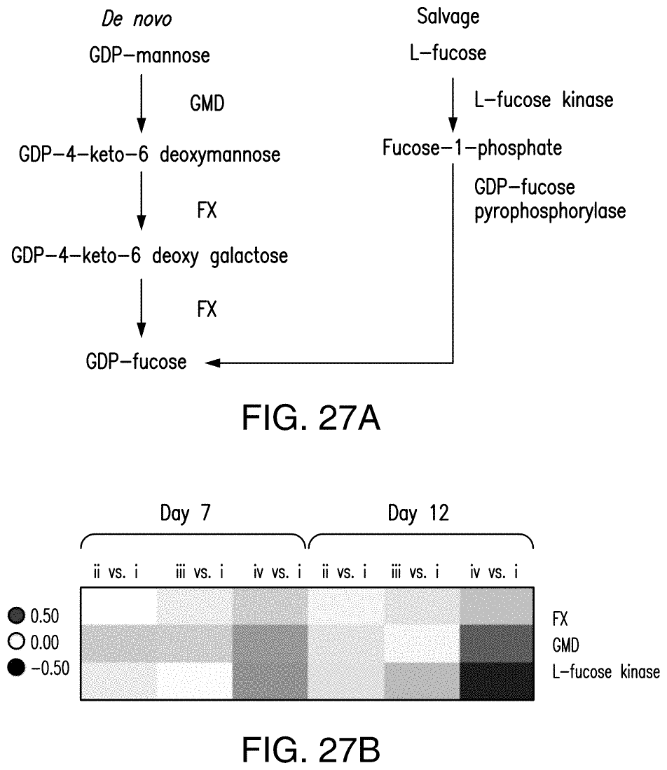

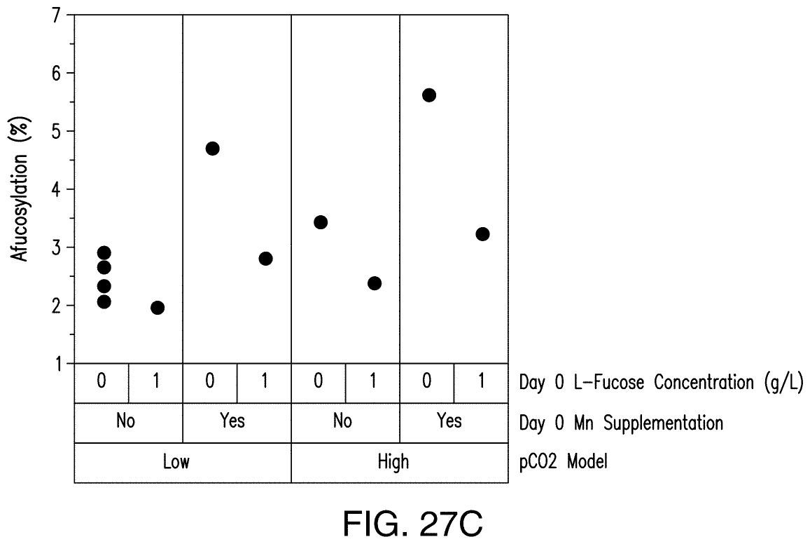

Untargeted proteomics was performed to uncover the underlying mechanism(s) behind the impact of Mn, media hold, and high pCO₂/Na⁺ on afucosylation. Production cultures were subjected to four different conditions that are expected to impact afucosylation to varying extents (FIG. 25A). PCA (FIG. 26B) showed a clear separation of the samples by day (7 versus 12) and by treatment (i.e., cell culture conditions). IPA indicated that pathways related to glucose and amino acid metabolism (glycolysis, gluconeogenesis, methionine degradation, and cysteine biosynthesis) were upregulated in the presence of Mn, media hold, and high pCO₂ (FIG. 26C). Amongst the glycolytic enzymes, fructose bisphosphate aldolase showed the highest upregulation in differential expression for case iv relative to case i (FIG. 26D). Increase in Na⁺ can increase pHᵢ and the activity of phosphofructokinase, the rate-limiting enzyme in the glycolysis pathway that converts fructose-6-phosphate (Fru-6-P) into fructose 1,6-bisphosphate (Fidelman et al., (1982), Am J Physiol, 242 (1), C87-93). The enhanced activity of phosphofructokinase may increase the conversion of Fru-6-P to fructose 1,6-biphosphate, thereby lowering the levels of Fru-6-P and upregulating the expression of fructose biphosphate aldolase. Since Fru-6-P is the precursor for GDP-mannose, which is an upstream precursor for GDP-fucose in the de novo synthesis pathway (FIG. 27A), a decline in Fru-6-P would lower the supply of GDP-mannose and GDP-fucose, and hence increase afucosylation.

4.3.6 GDP-Fucose Synthesis Pathway: Proteomic Analysis and L-Fucose Supplementation

To assess the possibility that GDP-fucose was impacted in cell culture conditions that generated higher mAb afucosylation (i.e., cases ii-iv), the differential expression of key enzymes in the de novo and salvage GDP-fucose synthesis pathways were examined (FIG. 27A). The downregulation of GMD and L-fucose kinase correlated positively with afucosylation level: expression of GMD and L-fucose kinase was lowest for the culture treatment with the highest afucosylation (case iv) (FIG. 27B). No consistent change in FX was observed for cases ii-iv relative to case i.

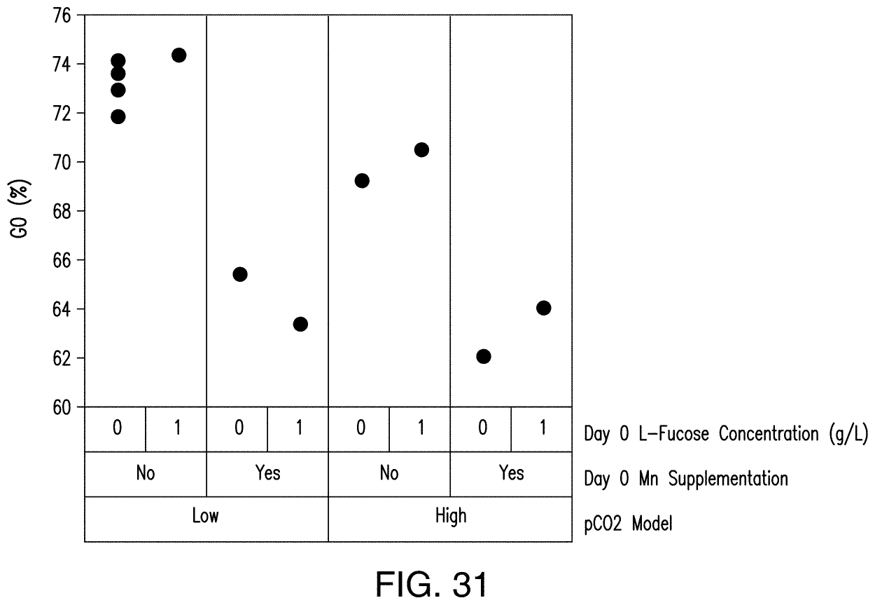

In previous studies, knocking out GMD or FX in CHO cell lines decreased GDP-fucose and increased afucosylation (Kanda et al., (2007), J Biotechnol, 130 (30), 300-310; Louie et al., (2016), Biotechnol Bioeng, 114 (3), 632-644) and afucosylation could be lowered by L-fucose supplementation to utilize the salvage pathway to synthesize GDP-fucose (Louie et al., (2016), Biotechnol Bioeng, 114 (3), 632-644). In light of the observations from proteomics here and knockout studies elsewhere, the higher afucosylation with high pCO₂ and/or supplemental Mn can be hypothesized to be at least partly attributed to limitations in GDP-fucose. To test this hypothesis, a full factorial DOE testing pCO₂, supplemental Mn, and supplemental L-fucose was performed (FIG. 27C). The effect of L-fucose on afucosylation saturated after ˜0.3 g/L in a previous titration study therefore, L-fucose supplementation at 1 g/L was selected in this evaluation. Supplementing L-fucose in conditions known to generate high afucosylation restored afucosylation to lower levels without impacting G0F (FIG. 31). Taken together, both proteomics and L-fucose supplementation results confirmed that GDP-fucose limitation contributes to the relatively high mAb afucosylation observed for the combination of high pCO₂, media hold, and supplemental Mn.

4.3.7 Proteomic Analysis: FUT8

In considering the key role of FUT8 on afucosylation, the differential expression of FUT8 in the four culture treatment cases i-iv was analyzed (FIG. 25A). FUT8 was downregulated and correlated negatively with afucosylation for cases ii-iv relative to case i (FIG. 28B). It is believed that this is the first study to demonstrate downregulation of FUT8 by high pCO₂, media hold, and supplemental Mn in CHO cells.

4.3.8 Proteomic Analysis: Other Glycosylation Enzymes, pHᵢ, Golgi pH, and Golgi Mn Concentration

The differential expression of glycosylation enzymes upstream (Man I, GnTII) and downstream (GalT3, GalT4, and GalT7) of FUT8 were assessed to determine if there were any additional bottlenecks in this segment of the glycosylation pathway (Hossler et al., (2009), Glycobiology, 19 (9), 936-949; Kremkow et al., (2018), Metabol Eng, 47, 134-142). There was minimal change for Man I and GnTII and inconsistent change for GalT3, GalT4, and GalT7 expression level across test cases and culture days (FIG. 28B). Even though these glycosylation enzymes did not show differential expression, proteomics cannot ascertain changes in enzyme activity. Activity of these enzymes may be impacted by pHᵢ, or by changes in intracellular Mn content because Mn is a cofactor for some (Rouiller et al., (2014), Biotechnol Prog, 30 (3), 571-583; Gramer et al., (2011), Biotechnol Bioeng, 108 (7), 1591-1602). Proteomics data support these theories, as discussed below.

NHE1, a Na⁺/H⁺ exchanger involved in pHᵢ regulation (Orlowski et al., (1997), J Biol Chem, 272(36), 22373-22376) and GPR89, a Golgi pH regulator (Maeda et al., (2008), Nature Cell Biology, 10 (10), 1135-1145), were upregulated and correlated positively with afucosylation and pCO₂/Na⁺ (FIG. 28B). These observations indicate that pHᵢ and Golgi pH were affected by pCO₂/Na⁺, consistent with the pHᵢ findings described above (FIG. 24).

ATP2A1, an ATP-dependent transporter of Mn into the Golgi (Baelen et al., (2004), Biochimica et Biophysica Acta, 1742(1-3), 103-112), was upregulated and correlated positively with afucosylation and pCO₂/Na⁺ (FIG. 28B). GPP130, a Golgi protein whose degradation depends solely on the intracellular Mn level (Mukhopadhyay et al., (2010), Molecular Biology of the Cell, 21, 1282-1292; Masuda et al., (2013), Synapse, 67 (5), 205-215; Venkat et al., (2017), Molecular Biology of the Cell, 28, 2569-2578), was downregulated and correlated negatively with afucosylation and pCO₂/Na⁺ (FIG. 28B). These results indicate that intracellular Mn level was highest in case iv relative to the other cases. The higher intracellular Mn level potentially increased the activity of GnTs and GalTs to favor an overall flux towards the afucosylated glycoforms. Thus, enhanced Mn transport and intracellular Mn level can contribute towards the higher afucosylation in the culture conditions of high pCO₂/Na+, supplemental Mn, and media hold.

It will be understood that the foregoing is only illustrative of the principles of the present disclosure, and that various modifications can be made by those skilled in the art without departing from the scope and spirit of the present disclosure. For example, but not by way of limitation, volume of the bioreactor used in the example can be between about 1 L and about 20,000 L (e.g., about 1 L, about 1.5 L, about 2 L, about 5 L, about 10 L, about 50 L, about 100 L, about 250 L, about 500 L, about 1000 L, about 2000 L, about 3000 L, about 4000 L, about 5000 L, about 6000 L, about 7000 L, about 8000 L, about 9000 L, about 10,000 L, about 11,000 L, about 12,000 L, about 13,000 L, about 14,000 L, about 15,000 L, about 16,000 L, about 17,000 L, about 18,000 L, about 19,000 L, or about 20,000 L). Furthermore, bioreactor configurations can be modified to adjust levels of pCO₂, medial hold duration, osmolality, Na+, Mn, temperature, pH, fucose, galactose, or combinations thereof.

### Example 5: Media Hold to Modulate Glycosylation

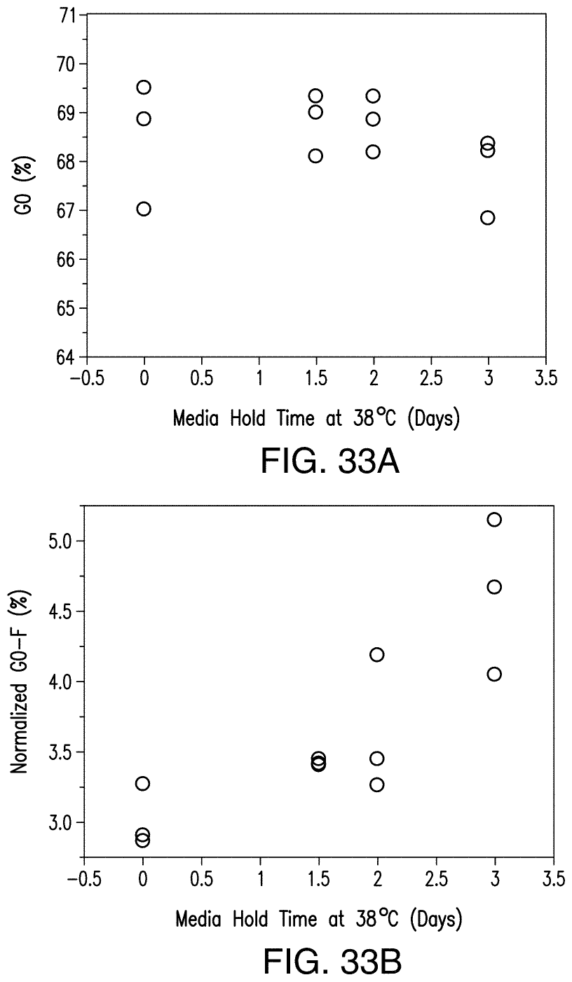

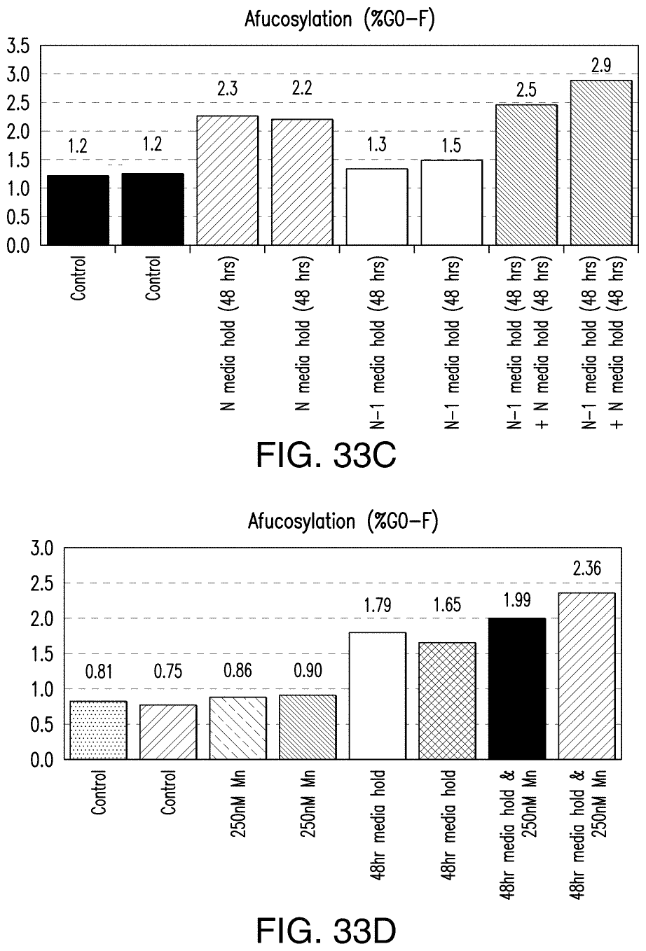

Media hold experiments were performed for Antibody VI to evaluate the changes in glycosylation (e.g., afucosylation and galactosylation) with increasing media hold time at elevated temperature in bioreactors. Media for production culture and expansion cultures prior to production were held at 38° C. for 0, 36, 48, and 72 hours in a bioreactor with air sparge, agitation, and pH control, prior to being used to inoculate the culture. FIGS. 33A-33B show the effects of media hold time at elevated temperature (38° C.) on glycosylation. The correlation between media hold time and G0 is shown in FIG. 33A. The correlation between media hold time and afucosylation (e.g., normalized G0-F) is shown in FIG. 33B. Furthermore, afucosylation increased with increased media hold time. As shown in FIG. 33B, the level of normalized G0-F showed a significant media hold time dependent increase when media hold was applied to the cell culture media. (FIG. 33A).

Media hold experiments were performed for Antibody III to evaluate the changes in afucosylation. To evaluate cumulative effects of media hold on afucosylation for Antibody III, four cases were tested in duplicate bioreactors. The production (N) and/or inoculum train (N−1) media were held at elevated temperature (˜37° C.) for 48 hours. Control case represents use of production and inoculum train media that were not held at elevated temperature prior to use in inoculating bioreactors. As shown in FIG. 33C, the results demonstrate that both N and N−1 media hold increase afucosylation (represented by % G0-F here) on their own. The largest increase in afucosylation was observed when N media hold was used in combination with N−1 media hold, thereby demonstrating the cumulative effect of media hold on afucosylation.

Media hold and Mn supplementation experiments were performed for Antibody III to evaluate the changes in afucosylation. To evaluate discrete and combinatory/synergistic effects of media hold and Mn supplementation on afucosylation for Antibody III, four cases were tested in duplicate bioreactors. The production medium was held at elevated temperature (˜37° C.) for 48 hours with/without 250 nM Mn supplemented. Control case represents use of production medium that was not held at elevated temperature prior to use in inoculating bioreactors and the lack of Mn supplementation. For the conditions tested, as shown in FIG. 33D, media hold showed a larger impact than Mn supplementation on afucosylation (represented by % G0-F). The largest increase in afucosylation was observed when media hold was used in combination with Mn supplementation. In particular, the largest increase in % G0-F was observed when 48 hr media hold was used in combination with 250 nM Mn supplementation (FIG. 33D). Exposure to 48 hr media hold increased the level of % G0-F. The level of % G0-F increased when 250 nM of Mn was supplemented into the media which was exposed to media hold.

The increase of normalized G0-F with media hold time (36 hour hold time) was also observed at 37° C. as shown in FIG. 21A. An interaction was also observed between media hold time, pCO₂, and Mn. The unexpected synergistic interaction resulted in an increase in normalized G0-F greater than the sum of the normalized G0-F increases observed for each single factor (FIG. 21A).

It will be understood that the foregoing is only illustrative of the principles of the present disclosure, and that various modifications can be made by those skilled in the art without departing from the scope and spirit of the present disclosure. For example, but not by way of limitation, volume of the bioreactor used in the example can be between about 1 L and about 20,000 L (e.g., about 1 L, about 1.5 L, about 2 L, about 5 L, about 10 L, about 50 L, about 100 L, about 250 L, about 500 L, about 1000 L, about 2000 L, about 3000 L, about 4000 L, about 5000 L, about 6000 L, about 7000 L, about 8000 L, about 9000 L, about 10,000 L, about 11,000 L, about 12,000 L, about 13,000 L, about 14,000 L, about 15,000 L, about 16,000 L, about 17,000 L, about 18,000 L, about 19,000 L, or about 20,000 L). Furthermore, bioreactor and their operations can be modified to adjust levels of pCO₂, media hold duration, culture duration, osmolality, Na+, Mn, cultivation temperature, fucose, galactose, or combinations thereof.

### Example 6: Galactose Addition to Modulate Glycosylation

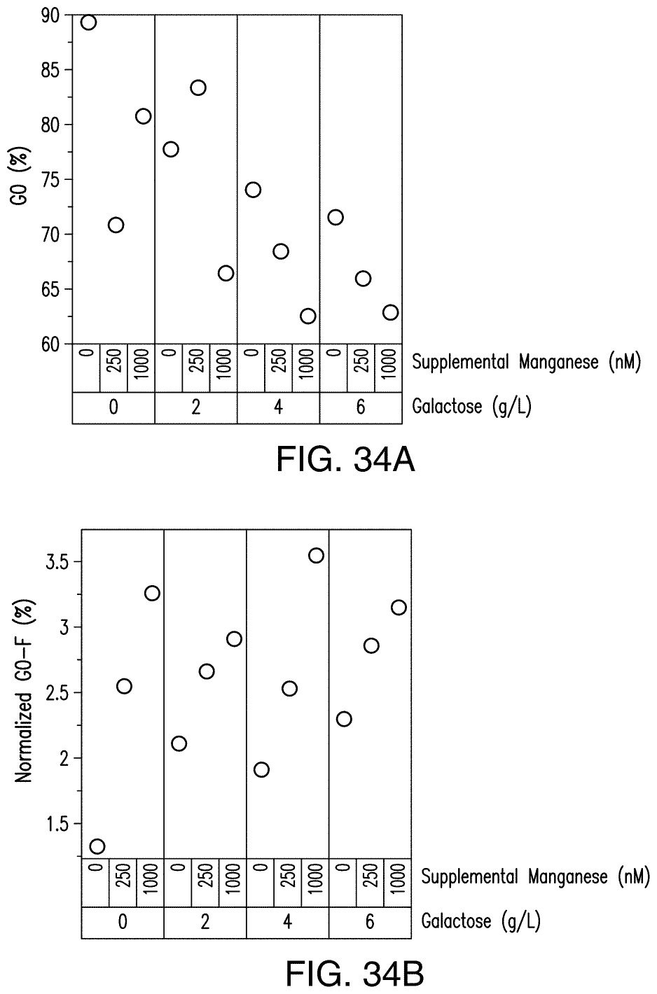

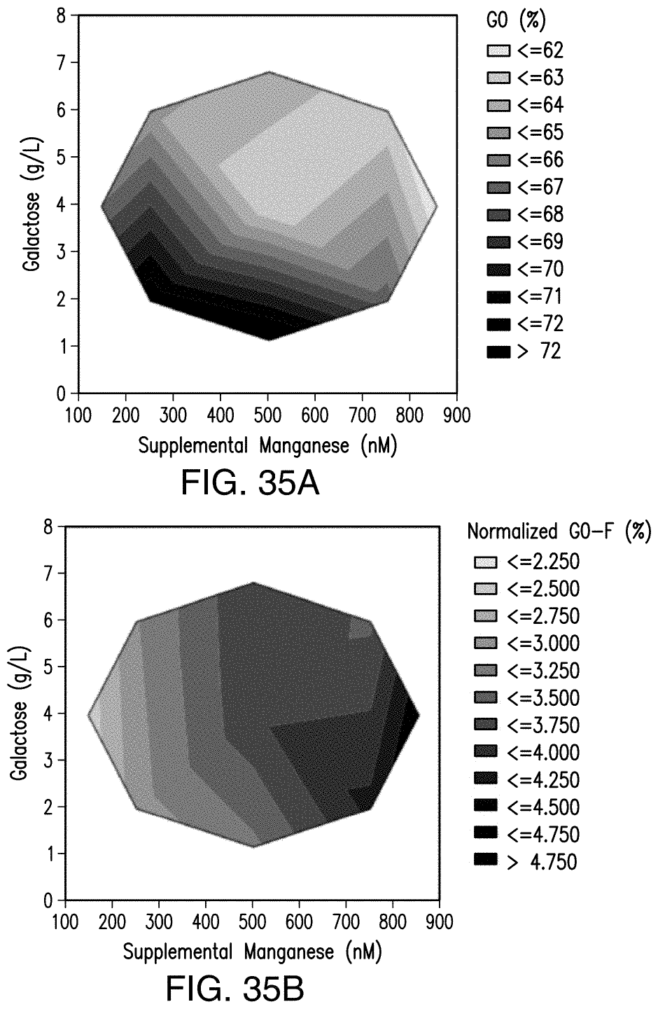

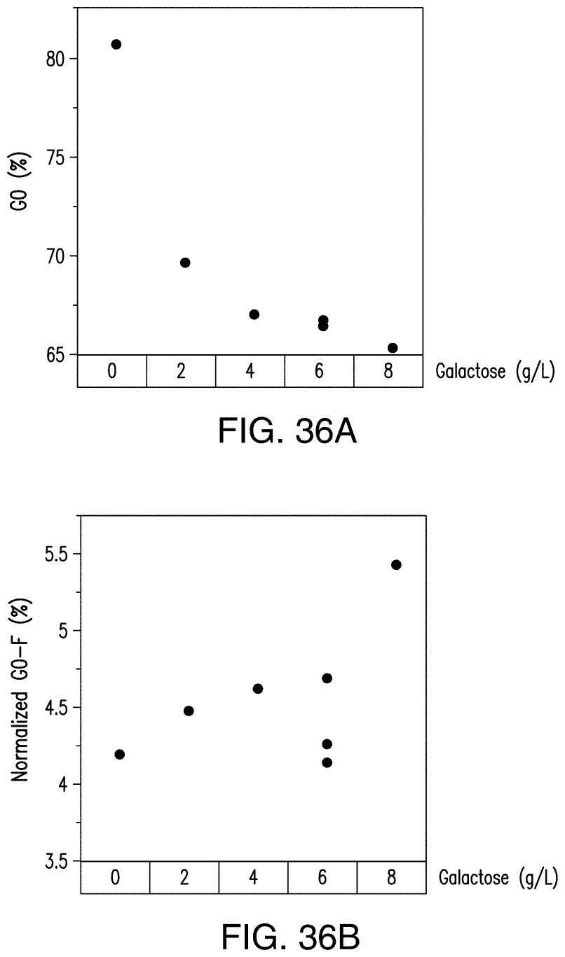

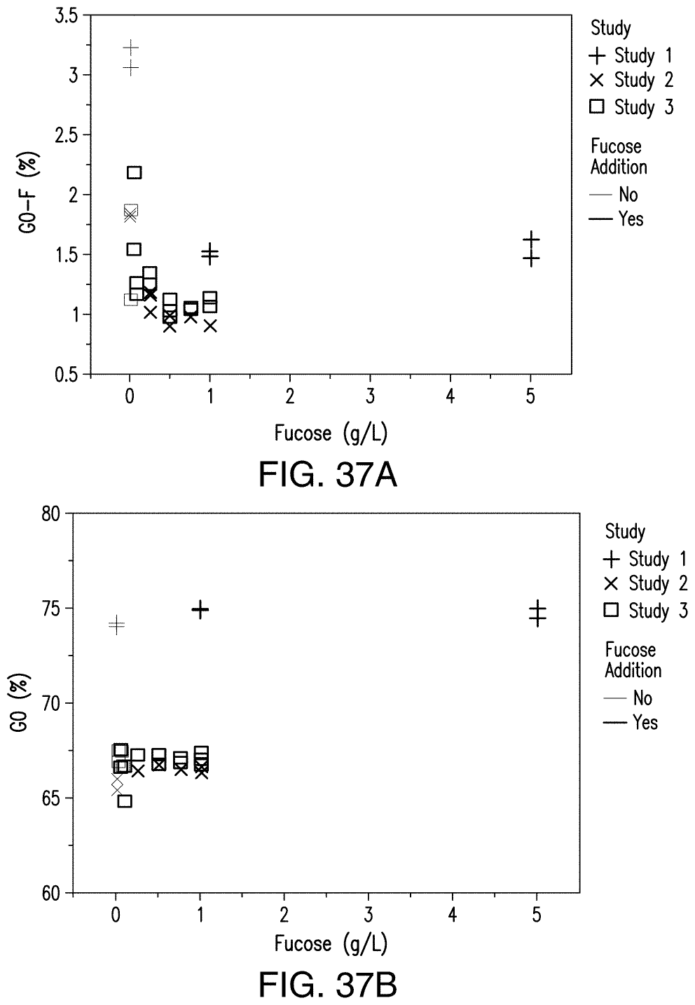

Three studies were performed to evaluate addition of galactose, Mn, and combinations thereof, on glycosylation of Antibody VI. The results of studies 1, 2, and 3 are shown in FIG. 34-FIG. 36. Galactose, or Mn, or combinations thereof can be adjusted to target a specific distribution of glycosylation (e.g., G0 and normalized G0-F). An increase in galactose level and/or Mn level resulted in lower agalactosylation (G0) and higher afucosylation (normalized G0-F). Additionally, at lower levels of galactose, G0 was more sensitive to changes in Mn level compared to higher levels of galactose. Similarly, at lower levels of Mn, G0 was more sensitive to changes in galactose level compared to higher levels of Mn. In particular, an unexpected synergistic decrease in % G0 was observed when different concentrations of galactose were added in combination with Mn supplementation (FIGS. 34A and 35A). Each galactose and Mn supplementation decreased the level of G0 in a dose-dependent manner. However, when both galactose and Mn were added together into culture media, the level of G0 significantly and synergistically decreased. The levels of G0-F showed relatively less change when both galactose and Mn were added together into culture media (FIGS. 34B and 35B).

### Example 7: Fucose Supplementation and Cultivation Temperature to Modulate Glycosylation

7.1 Introduction

This example summarizes effects of fucose supplementation on glycosylation. Higher fucose supplementation levels and/or earlier fucose supplementation results in a larger decrease in afucosylation (e.g., G0-F). An interaction was observed with cultivation temperature, wherein a larger impact was observed on afucosylation at lower culture temperatures.

7.2 Evaluation of Fucose Concentration Effects of fucose addition on glycosylation was evaluated in three Antibody VI studies. FIGS. 378A-378B shows the impact of fucose concentration on afucosylation (e.g., G0-F) and galactosylation (e.g., G0). An increase in fucose level resulted in higher afucosylation (normalized G0-F).

7.3 Evaluation of Fucose Addition Timing

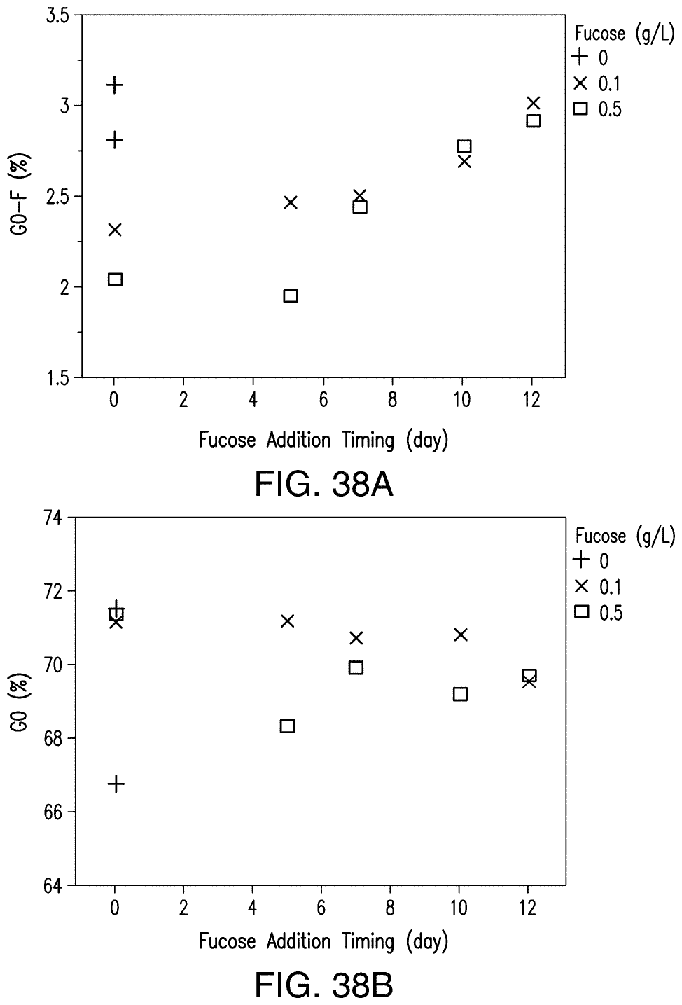

The effects of fucose addition timing on glycosylation was evaluated at two different fucose levels with Antibody VI. Fucose was added as a post-inoculation addition to the production culture at five different time points. FIGS. 38A-38B show the impact of fucose addition timing on afucosylation (e.g., G0-F) and galactosylation (e.g., G0). Larger decreases in G0-F were observed with earlier fucose addition. G0 was not affected by fucose addition timing.

7.4 Evaluation of Fucose Supplementation and Interaction with Temperature

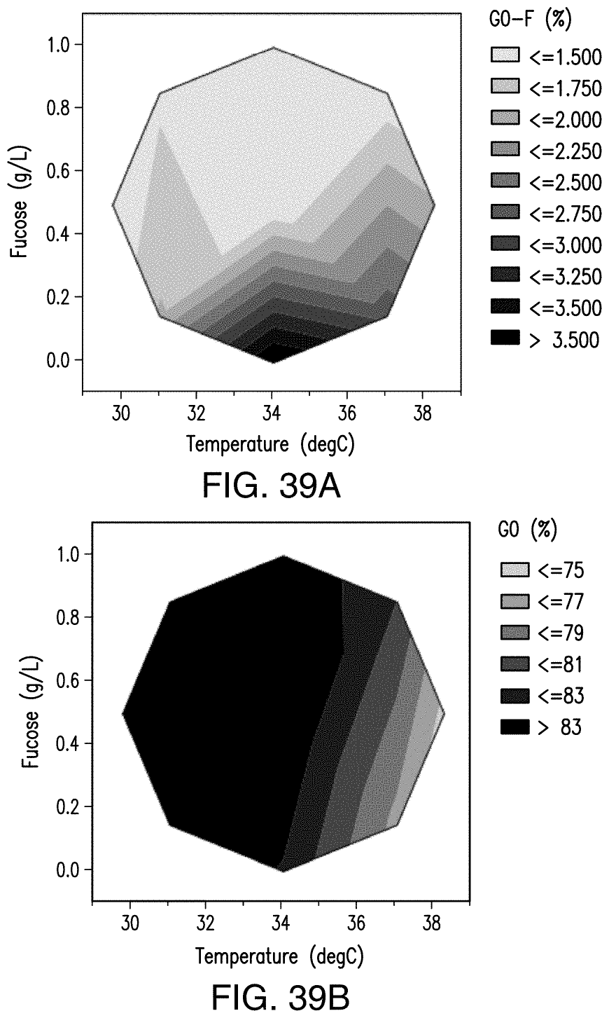

To assess effects of fucose concentration and temperature, as well as the interaction between fucose and temperature, on glycosylation, a central composite design study was performed with Antibody VI. Fucose was added as a post-inoculation addition on day 0 of the production culture. FIGS. 39A-39B show the impact of fucose and temperature on afucosylation (e.g., G0-F) and galactosylation (e.g., G0). Increasing fucose concentration and decreasing temperature resulted in lower afucosylation (e.g., G0-F) levels. An unexpected interaction between fucose and temperature was observed, with fucose having a larger impact on afucosylation (e.g., G0-F) at lower temperatures than at higher temperatures. As shown in FIGS. 39A-39B, larger quantities of fucose was required at elevated temperatures in order to reach the same G0-F level. Decreasing temperature resulted in higher G0 levels.

What is claimed is:

1. A method for modulating the glycosylation pattern of a glycoprotein of interest in a cell culture, comprising modulating the following parameters, either alone or in any combination, in a cell culture medium, and/or, in a cell culture environment:
a. a Mn concentration from about 1 nM to about 20000 nM in a high partial pressure C0₂ (pC0₂) condition;
b. a Mn concentration from about 1 nM to about 30000 nM in a low pC0₂ condition;
c. a pC0₂ from about 10 mmHg to about 250 mmHg;
d. a pre-inoculation cell culture media hold duration from about 0 hrs to about 120 hrs at a temperature of about 25° C. to 39° C.;
e. a cell culture duration from about 0 days to about 150 days;
f. a Na+ concentration from about 0 mM to about 300 mM;
g. an osmolality from about 250 mOsm/kg to about 550 mOsm/kg;
h. a galactose concentration from about 0 mM to about 60 mM;
i. a fucose concentration from about 0 mM to about 60 mM; and
j. a cultivation temperature from about 29° C. to about 39° C.

2. The method of claim 1, wherein the duration of pC0₂ modulation covers at least the first half of the cell culture duration.

3. The method of claim 1, wherein the glycoprotein of interest is a recombinant protein.

4. The method of claim 1, wherein the recombinant protein is an antibody or antibody fragment, a scFv (single-chain variable fragment), BsDb (bispecific diabody), scBsDb (single-chain bispecific diabody), scBsTaFv (single-chain bispecific tandem variable domain), DNL-(Fab)3 (dock-and-lock trivalent Fab), sdAb (single-domain antibody) and BssdAb (bispecific single-domain antibody).

5. The method of claim 1, wherein the antibody is a chimeric, a humanized or a human antibody.

6. The method of claim 1, wherein the antibody is an anti-CD20 antibody.

7. The method of claim 1, wherein the anti-CD20 antibody is ocrelizumab.

8. The method of claim 4, wherein the antibody or antibody fragment exhibits:
i) a % G0-F (percent afucosylated glycoprotein) between about 0% to about 20%; about 1% to about 15%; about 1% to about 10%; or about 1% to about 8%; or, a normalized % G0-F between about 0% to about 20%; about 1% to about 15%; about 1% to about 10%; or about 1% to about 8%; and/or,
ii) a % G0 (percent agalactosylated glycoprotein) between about 40% to about 90%; about 50% to about 90%; about 55% to about 85%; or about 60% to about 80%.

9. The method of claim 4, wherein the glycosylation is modulated to achieve:
a. an increased afucosylation (e.g., G0-F (afucosylated G0)), while decreasing agalactosylation (e.g., G0 (fucosylated, agalactosylated G0)); or,
b. a decreased afucosylation (e.g., G0-F), while increasing agalactosylation (e.g., G0); or,
c. an increased or decreased afucosylation (e.g., G0-F) without impacting agalactosylation (e.g., G0); or,
d. an increased or decreased agalactosylation (e.g., G0) without impacting afucosylation (e.g., G0-F).

10. The method of claim 1, wherein modulation of the Mn concentration comprises determining the Mn content in cell culture raw materials and selecting raw material lots to modulate the Mn concentration.

11. The method of claim 1, wherein modulation of the Mn concentration comprises (i) controlling materials that come into contract with cell culture media or cell culture; or (ii) accounting for the concentration of leached Mn in cell culture media or during cell culture; or a combination of (i) and (ii) to modulate the Mn concentration.

12. The method of claim 11, wherein the leached Mn is produced by contact of the cell culture and/or cell culture media with: (i) a filter; (ii) a media preparation, hold, or culture vessel; or (iii) combinations of (i) and (ii).

13. The method of claim 1, wherein the cell culture medium is a basal medium, a reconstituted medium, a feed medium, a hydrolysate, a supplement, serum or an additive.

14. The method of claim 1, wherein the cell culture medium is supplemented: (1) during the production stage of the cell culture; or (2) prior to the production stage of the cell culture.

15. The method of claim 1, wherein the cell culture medium comprises one or more of: Mn, fucose, galactose and/or Na+, and wherein the supplementation is based on a pre-defined schedule or criteria.

16. The method of claim 1, wherein the one or more of the Mn, fucose, galactose and Na+ is supplemented as a bolus, as an intermittent supplement, as a continuous supplement, as a semi-continuous supplement, as a feedback loop-based supplement, or as a combination of one or more of thereof.

17. The method of claim 1, wherein the cell culture medium supplement consists essentially of one or more of: i) Mn; ii) fucose; iii) galactose; and/or iv) Na+.

18. The method of claim 1, wherein the modulation of the Mn concentration comprises employing a cell culture media pH of about 6.1 to about 7.3; or about 6.3 to about 7.3 prior to High Temperature Short Time (HTST) heat treatment.

19. The method of claim 1, wherein modulation of the glycosylation pattern of the glycoprotein of interest comprises modulating the pCO₂.

20. The method of claim 1, wherein the pCO₂ modulation comprises: (1) establishing a low pCO₂ culture; or (2) occurs at day 0 of the culture.

21. The method of claim 1, wherein modulation of the glycosylation pattern of the glycoprotein of interest comprises modulating the duration of the cell culture, wherein the duration of the cell culture is about 0 days to about 150 days; about 0 days to about 15 days; about 0 days to about 12 days; 0 days to about 7 days; or about 0 days to about 5 days.

22. The method of claim 1, wherein modulation of the glycosylation pattern of the glycoprotein of interest comprises modulating the Na+ concentration, wherein the Na+ concentration is about 0 mM to about 300 mM; is about 20 mM to about 20 mM; about 30 mM to about 150 mM; or about 40 mM to about 130 mM.

23. The method of claim 1, wherein modulation of the glycosylation pattern of the glycoprotein of interest comprises modulating the osmolality, wherein the osmolality of is about 250 mOsm/kg to about 550 mOsm/kg; about 300 mOsm/kg to about 450 mOsm/kg; or about 325 mOsm/kg to about 425 mOsm/kg.

24. The method of claim 1, wherein modulation of the glycosylation pattern of the glycoprotein of interest comprises modulating the galactose concentration, wherein the galactose concentration is about 0 mM to about 60 mM or about 0 mM to about 50 mM.

25. The method of claim 1, wherein modulation of the glycosylation pattern of the glycoprotein of interest comprises modulating the fucose concentration, wherein the fucose concentration is about 0 mM to about 60 mM; 0 mM to about 40 mM; about 0 mM to about 20 mM; or about 0 mM to about 10 mM.

26. The method of claim 1, wherein modulation of the glycosylation pattern of the glycoprotein of interest comprises modulating the cell culture temperature, wherein the cell culture temperature is about 29° C. to about 39° C.; about 30° C. to about 39° C.; about 31° C. to about 38° C.; or about 34° C. to about 38° C.

27. The method of claim 1, wherein the cell culture temperature is modulated: (1) prior the production stage of the cell culture; or (2) based on a pre-defined schedule or criteria.

28. The method of claim 1, wherein the cell culture comprises eukaryotic cells.

29. The method of claim 28, wherein the eukaryotic cells are CHO cells.

30. The method of claim 1, wherein the cell culture is in a bioreactor including but not limited to: a single use technology (SUT) bag or bioreactor; a WAVE bioreactor; a stainless steel bioreactor; a flask; a tube and a chamber.

31. The method of claim 1, wherein the volume of the cell culture is from 1 mL to 35,000 L.

32. A method to prepare a cell culture media, a feed media, a hydrolysate, or an additive comprising one or more step(s) of modulating:
a. the Mn concentration in a high partial pressure C0₂ (pC0₂) culture from about 1 nM to about 20000 nM;
b. the Mn concentration in a low pC0₂ culture from about 1 nM to about 30000 nM;
c. the pC0₂ from about 10 mmHg to about 250 mmHg;
d. the pre-inoculation cell culture media hold duration from about 0 hrs to about 120 hrs;
e. the cell culture duration from about 0 days to about 150 days;
f. the Na+ concentration from about 0 mM to about 300 mM;
g. the osmolality from about 250 mOsm/kg to about 550 mOsm/kg;
h. the galactose concentration from about 0 mM to about 60 mM;
i. the fucose concentration from about 0 mM to about 60 mM; and
j. the cultivation temperature from about 29° C. to about 39° C.;
wherein the cell culture media, feed media, hydrolysate, or additive modulates the glycosylation pattern of a glycoprotein of interest.

33. A cell culture composition comprising,
a. a host cell engineered to express a glycoprotein of interest; and
b. a cell culture and/or cell culture media modulated to target one or more predetermined parameter selected from:
i. the Mn concentration in a high partial pressure C0₂ (pC0₂) culture from about 1 nM to about 20000 nM;
ii. the Mn concentration in a low pC0₂ culture from about 1 nM to about 30000 nM;
iii. the pC0₂ from about 10 mmHg to about 250 mmHg;
iv. the pre-inoculation cell culture media hold duration from about 0 hrs to about 120 hrs;
v. the cell culture duration from about 0 days to about 150 days;
vi. the Na+ concentration from about 0 mM to about 300 mM;
vii. the osmolality from about 250 mOsm/kg to about 550 mOsm/kg;
viii. the galactose concentration from about 0 mM to about 60 mM;
ix. the fucose concentration from about 0 mM to about 60 mM; and
x. the cultivation temperature from about 29° C. to about 39° C.

34. A method for producing a glycoprotein of interest in a cell culture, comprising:
a. subjecting a cell culture medium suitable for cultivating a eukaryotic cell to the method according to claim 1,
b. inoculating the modulated cell culture medium with the eukaryotic cell that expresses the recombinant protein;
c. cultivating the eukaryotic cell so that the recombinant protein is expressed.

35. A method of modulating the glycosylation of a glycoprotein of interest, the method comprising:
a. assaying cell culture media to determine if the manganese concentration of the cell culture media falls within a targeted range; and
b. culture a host cell engineered to express the glycoprotein of interest in the cell culture media falling within the targeted range;
wherein the glycosylation of glycoproteins of interest is modulated as compared to the glycosylation of glycoproteins of interest expressed by the host cell in culture media falling outside the targeted range of manganese concentrations.

36. A cell culture composition comprising,
a. a cell culture media assayed to determine if the manganese concentration of the cell culture media falls within a targeted range; and
b. a host cell engineered to express a glycoprotein of interest.

37. A composition comprising a glycoprotein of interest, wherein the preparation comprises:
a. a cell culture media assayed to determine if the manganese concentration of the cell culture media falls within a targeted range;
b. a host cell engineered to express a glycoprotein of interest; and
c. the glycoprotein of interest.
